(19) United States
(12) Patent Application Publication
(10) Pub. No.: US 2004/0203023 A1
(43) Pub. Date: Oct. 14, 2004

(54) Proteins, genes and their use for diagnosis and treatment of breast cancer

(76) Inventor: Herath Mudiyanselage Athula Chandrasiri Herath, Abingdon (GB)

Correspondence Address: KLAUBER & JACKSON, 411 HACKENSACK AVENUE, HACKENSACK, NJ, 07601

(21) Appl. No.: 10/700,340

(22) Filed: Nov. 3, 2003

Related U.S. Application Data
(63) Continuation of application No. PCT/GB02/02022, filed on May 2, 2002.

(30) Foreign Application Priority Data
May 2, 2001 (GB) 0110790.3
Jul. 27, 2001 (GB) 0118385.4
Aug. 14, 2001 (GB) 0119791.2
Aug. 16, 2001 (GB) 0120045.0
Nov. 22, 2001 (GB) 0128062.7

(51) Int. Cl. C12Q 1/68; G01N 33/574

(52) U.S. Cl. 435/6; 435/7.23

(57) ABSTRACT

The present invention relates to the identification of polypeptides, proteins and protein isoforms that are associated with breast cancer and its onset and development, and of genes encoding the same, and to their use for e.g., clinical screening, diagnosis, prognosis, therapy and prophylaxis, as well as for drug screening and drug development.

### RELATED APPLICATIONS

The present application is a Continuation of co-pending PCT Application No. PCT/GB02/02022 filed May 2, 2002, which in turn, claims priority from Great Britain Application Serial No. 0110790.3, filed on May 2, 2001, Great Britain Application No. 0118385.4, filed on Jul. 27, 2001, Great Britain Application Serial No. 0119791.2, filed on Aug. 14, 2001, Great Britain Application Serial No. 0120045.0, filed on Aug. 16, 2001 and Great Britain Application Serial No. 0128062.7, filed on Nov. 22, 2001. Applicants claim the benefits of 35 U.S.C. §120 as to the PCT application and priority under 35 U.S.C. § 119 as to the said Great Britain applications, and the entire disclosures of all applications are incorporated herein in their entireties.

### INTRODUCTION

The present invention relates to the identification of polypeptides, proteins and protein isoforms that are associated with breast cancer and its onset and development, of genes encoding the same, and to their use for e.g., clinical screening, diagnosis, prognosis, therapy and prophylaxis, as well as for drug screening and drug development.

### BACKGROUND OF THE INVENTION

Breast cancer is the most frequently diagnosed non-skin cancer among women in the United States. It is second only to lung cancer in cancer-related deaths. Approximately 180,000 new cases of breast cancer were diagnosed in 1997 in the US, and about 44,000 women were expected to die from the disease. A report from the National Cancer Institute (NCI) estimates that about 1 in 8 women in the United States (approximately 12.8 percent) will develop breast cancer during her lifetime. This estimate is based on data from NCI's Surveillance, Epidemiology, and End Results Program (SEER) publication SEER Cancer Statistics Review 1973-1997 and is based on cancer rates from 1995 through 1997. (National Cancer Institute, www.cancernet.nci.nih.gov, USA, 1999). In the UK, breast cancer is by far the commonest cancer for women, with 34,600 new cases in 1998 (Cancer Research Campaign, www.crc.org.uk, UK, 2000). Ninety-nine percent of breast cancers occur in women. The annual cost of breast cancer treatment in the United States is approximately $10 billion (Fuqua, et. al.1998, American Association for Cancer Research, www.aacr.org, USA). Breast cancer incidence has been rising over the past five decades, but recently it has plateaued. This may reflect a period of earlier detection of breast cancers by mammography. A number of established factors can increase a woman's risk of having the disease. These include older age, history of prior breast cancer, significant radiation exposure, strong family history of breast cancer, upper socioeconomic class, nulliparity, early menarche, late menopause, or age at first pregnancy greater than 30 years. Prolonged use of oral contraceptives earlier in life appears to increase risk slightly. Prolonged postmenopausal oestrogen replacement increases the risk 20 to 40%. It has been speculated that a decrease in the age at menarche, changing birth patterns, or a rise in the use of exogenous estrogens has contributed to the increase in breast cancer incidence (Fuqua, et. al. 1998, American Association for Cancer Research, www.aacr.org, USA).

Causes of Breast Cancer

Breast cancer is a heterogeneous disease. Although female hormones play a significant role in driving the origin and evolution of many breast tumours, there are a number of other recognised and unknown factors involved. Perturbations in oncogenes identified include amplification of the HER-2 and the epidermal growth factor receptor genes, and overexpression of cyclin D1. Overexpression of these oncogenes has been associated with a significantly poorer prognosis. Similarly, genetic alterations or the loss of tumour suppressor genes, such as the p53 gene, have been well documented in breast cancer and are also associated with a poorer prognosis. Researchers have identified two genes, called BRCA1 and BRCA2, which are predictive of premenopausal familial breast cancer. Genetic risk assessment is now possible, which may enhance the identification of candidates for chemoprevention trials (Fuqua, et. al. 2000, American Association for Cancer Research, www.aacr.org, USA).

Diagnosis

Early diagnosis of breast cancer is vital to secure the most favourable outcome for treatment. Many countries with advanced healthcare systems have instituted screening programmes for breast cancer. This typically takes the form of regular x-ray of the breast (mammography) during the 50-60 year old age interval where greatest benefit for this intervention has been shown. Some authorities have advocated the extension of such programmes beyond 60 and also to the 4049 age group. Health authorities in many countries have also promoted the importance of regular breast self-examination by women. Abnormalities detected during these screening procedures and cases presenting as symptomatic would normally be confirmed by aspiration cytology, core needle biopsy with a stereotactic or ultrasound technique for nonpalpable lesions, or incisional or excisional biopsy. At the same time other information relevant to treatment options and prognosis, such as oestrogen (ER) and progesterone receptor (PR) status would be determined (National Cancer Institute, USA, 2000, Breast Cancer PDQ, www.nci.nih.gov).

Disease Staging and Prognosis

Staging is the process of finding out how far the cancer has spread. The staging system of the American Joint Committee on Cancer (AJCC), also known as the TNM system, is the one used most often for breast cancer. The TNM system for staging gives three key pieces of information:

The letter T followed by a number from 0 to 4 describes the tumour's size and spread to the skin or chest wall under the breast. A higher number means a larger tumour and/or more spread to tissues near the breast.

The letter N, followed by a number from 0 to 3, indicates whether the cancer has spread to lymph nodes near the breast and, if so, whether the affected nodes are adhered to other structures under the arm.

The letter M, followed by a 0 or 1, shows whether the cancer has metastasised to other organs of the body or to lymph nodes that are not next to the breast.

To make this information somewhat clearer, the TNM descriptions can be grouped together into a simpler set of stages, labelled stage 0 through stage IV (0-4). In general, the lower the number, the less the cancer has spread. A higher number, such as stage IV (4), means a more serious cancer. (American Cancer Society, 2000, USA, www.cancer.org.uk)

Breast Cancer Survival by Stage

| Stage | 5-year relative survival rate |
|---|---|
| 0 | 100% |
| I | 98% |
| IIA | 88% |
| IB | 76% |
| IIIA | 56% |
| IIIB | 49% |
| IV | 16% |

(American Cancer Society, 2000, USA, www.cancer.org.uk)

Although anatomic stage (size of primary tumour, axillary lymph node involvement) is an important prognostic factor, other characteristics may have predictive value. For example studies from the National Surgical Adjuvant Breast and Bowel Project (NSABP) and the International Breast Cancer Study Group (IBCSG) have shown that tumour nuclear grade and histologic grade, respectively, are important indicators of outcome following adjuvant therapy for breast cancer. There is substantial evidence that oestrogen receptor status and measures of proliferative capacity of the primary tumour (thymidine labelling index or flow cytometric measurements of S-phase and ploidy) may have important independent predictive value. In stage II disease, the PR status may have greater prognostic value than the ER status. Tumour vascularisation, c-erbB-2, c-myc, p53 expression, and lymphatic vessel invasion may also be prognostic indicators in patients with breast cancer (National Cancer Institute, USA, 2000, Breast Cancer PDQ, www.nci.nih.gov and references therein). The Need for Improved Diagnostic Tools in Breast Cancer Detection and Therapy

Although there are signs that benefits are accruing from the more vigorous application of existing screening methods such as targeted mammography and self-examination combined with public awareness programs, these approaches have limitations in the drive to detect breast cancer as early as possible. An important factor limiting the spread of mammographic screening and its extension to wider age groups is cost. Mammography requires expensive x-ray equipment and highly trained specialists to operate it and interpret mammograms. In addition, suspicious lesions detected by mammography currently need to be confirmed or cleared as benign by biopsy. This is an invasive procedure that requires subsequent expert histological examination and interpretation, and can delay definitive diagnosis. Once breast cancer has been diagnosed, the success of therapeutic interventions such as surgery, radiation and chemotherapy in stabilising or eliminating the disease can be difficult to establish. It can be particularly difficult to determine the extent of any residual disease in patients during remission and to make the important early discovery of any relapse into active disease. Both screening for and confirming the presence of breast cancer, and monitoring response to therapy, would be greatly aided by the application of a reliable and sensitive test that could detect the disease in serum samples.

Serum Protein Changes in the Detection of Disease

There are two types of changes in serum protein patterns that can potentially aid diagnosis and disease monitoring. The first of these is the detection in serum of novel proteins, not normally present, that have been shed into the serum from the cancer cells. The second type of change that can be of diagnostic significance is the detection of specific reactive proteins in the serum produced by the body in response to the disease. An example of a protein that can be shed into the serum by some breast cancer cells is a fragment of the growth factor receptor known as c-erbB2/HER2/neu, which is present in small amounts on the surface of normal breast cells and at much higher levels in some breast cancers (Payne et al., 2000, Clin. Chem. 46:175-182). A second example of a protein shed into serum by a cancer that has diagnostic or prognostic significance is prostate serum antigen or PSA, which is used in the diagnosis and monitoring of prostate cancer (Fowler et al., 2000, J. Urol. 163:813-818). A further example of a protein shed into serum by several types of cancer that can be of diagnostic or prognostic significance is carcino-embryonic antigen or CEA (Lumachi et al., 1999, Anticancer Res, SC: 4485-4489). The current value of these markers for diagnosis is limited by their lack of specificity and sensitivity, and these is a need to discover new markers that can better satisfy these criteria.

A number of reactive proteins collectively termed acute phase proteins, show a dramatic increase or decrease in concentration in serum in response to early “alarm” inflammatory mediators such as IL-1 released in response to tissue injury including cancer, or infection. An example of a reactive protein present in serum in response to disease that has diagnostic or prognostic significance is serum amyloid A or SAA in rheumatoid arthritis (Cunnane et al., 2000, J. Rheumatol. 27:56-63). Sensitive detection of selected examples of such proteins could also assist in the diagnosis of breast cancer. Due to the high rates at which other disorders co-occur with breast cancer, the time-consuming nature of existing, largely inadequate tests and their expense, it would be highly desirable to measure a substance or substances in samples of serum, blood or urine that would lead to a positive diagnosis of breast cancer or that would help to exclude breast cancer from the differential diagnosis.

Therefore a need exists to identify breast cancer associated proteins as sensitive and specific biomarkers for the diagnosis, to assess severity, to predict the outcome of breast cancer in living subject, and to monitor the treatment of breast cancer. Additionally, these is a clear need for new therapeutic agents for breast cancer that work quickly, potently, specifically, and with fewer side effects.

### SUMMARY OF THE INVENTION

The present invention provides methods and compositions for clinical screening, diagnosis and treatment of breast cancer, for monitoring the effectiveness of breast cancer treatment, for selecting participants in clinical trials, for identifying patients most likely to respond to a particular therapeutic treatment and for screening and development of drugs for treatment of breast cancer.

Therefore, the invention provides a method for screening or diagnosis of breast cancer in a subject, for determining the stage or severity of breast cancer in a subject, for identifying a subject at risk of developing breast cancer, or for monitoring the effect of therapy administered to a subject having breast cancer, said method comprising:

(a) analysing a test biological sample from the subject by two-dimensional electrophoresis to generate a two-dimensional array of features, said array comprising one or more of the following Breast Cancer Associated Features (BFs) as defined herein and

(b) comparing the abundance of the one or more BFs in the test biological sample with the abundance of the one or more BFs in a biological sample from one or more subjects free from breast cancer, or with a previously determined reference range for that feature in subjects free from breast cancer, or with the abundance at least one Expression Reference Feature (ERF) in the test sample.

Another aspect of the invention provides a method for screening or diagnosis of breast cancer in a subject, for determining the stage or severity of breast cancer in a subject, for identifying a subject at risk of developing breast cancer, or for monitoring the effect of therapy administered to a subject having breast cancer, as described above, wherein the method comprises quantitative detection of a cluster of Breast Cancer-Associated features (BFs).

These methods are also suitable for clinical screening, prognosis, monitoring the results of therapy, identifying patients most likely to respond to a particular therapeutic treatment, for drug screening and development, and identification of new targets for drug treatment.

The methods described herein can be performed on any biological sample including, without limitation, serum, blood, plasma or tissue homogenate. Preferably the methods are conducted on serum samples.

A further aspect of the invention provides methods for diagnosis of breast cancer that comprise detecting in a test biological sample the presence or level of at least one Breast Cancer-Associated Protein Isoform (BPI), disclosed herein or any combination thereof, in particular a cluster of BPIs as described herein. These methods are also suitable for clinical screening, prognosis, monitoring the results of therapy, identifying patients most likely to respond to a particular therapeutic treatment, drug screening and development, and identification of new targets for drug treatment.

An additional aspect of the invention provides antibodies, e.g. monoclonal, polyclonal, chimeric and humanised antibodies capable of immunospecific binding to a BPI.

Another aspect of the invention provides a preparation comprising an isolated BPI, i.e., a BPI free from polypeptides, proteins or protein isoforms having a significantly different isoelectric point or a significantly different apparent molecular weight from the BPI.

A further aspect of the invention provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

A further aspect of the invention provides methods of treating breast cancer, comprising administering to a subject a therapeutically effective amount of an agent that modulates (e.g., upregulates or downregulates) the expression or activity (e.g. enzymatic or binding activity), or both, of a BPI in subjects having breast cancer, in order to prevent or delay the onset or development of breast cancer, to prevent or delay the progression of breast cancer, or to ameliorate the symptoms of breast cancer. Another aspect of the invention provides methods of screening for agents that modulate (e.g., upregulate or downregulate) a characteristic of a BF, BPI, or a BPI-related polypeptide, such as the expression or the activity e.g. enzymatic or binding activity, of the BF, BPI, or a BPI-related polypeptide.

### BRIEF DESCRIPTION OF THE FIGURES

FIG. 1 is a flow chart depicting the characterisation of a BF and relationship of a BF and BPI. A BF may be further characterised as or by a BPI having a particular peptide sequence associated with its pI and MW. As depicted herein, a BF may comprise one or more BPIs, which have indistinguishable pIs and MWs using the Preferred Technology, but which have distinct peptide sequences. The peptide sequence(s) of the BPI can be utilised to search database(s) for previously identified proteins comprising such peptide sequence(s). It can be ascertained whether a commercially available antibody exists that may recognise the previously identified protein and/or a member of its protein family.

FIG. 2 is an image obtained from 2-dimensional electrophoresis of depleted serum representing a combination of normal serum and serum taken from subjects having breast cancer, which has been annotated to identify eleven landmark features, designated DS1, DS2, DS4, DS5, DS6, DS8, DS9, DS10, DS11, DS12, and DS13.

FIG. 3 is a Venn diagram depicting the number of BFs identified in serum samples taken from individuals with primary (Venn position A) and metastatic (Venn position C) breast cancer disease compared with serum samples taken from individuals with no breast cancer disease. An overlap of BFs identified in both breast cancer disease serum sample sets (Venn position B) was also identified.

### DETAILED DESCRIPTION OF THE INVENTION

The invention described in detail below provides methods and compositions for clinical screening and diagnosis of breast cancer in a mammalian subject for identifying patients most likely to respond to a particular therapeutic treatment, for monitoring the results of breast cancer therapy, for drug screening and drug development. The invention also encompasses the administration of therapeutic compositions to a mammalian subject to treat or prevent breast cancer. The mammalian subject may be a non-human mammal, but is preferably human, more preferably a human adult, e.g. a human subject at least 21 years old.

For clarity of disclosure, and not by way of limitation, the invention will be described with respect to the analysis of serum samples. However, as one skilled in the art will appreciate, the assays and techniques described below can be applied to other types of biological samples, including a body fluid (for example but without limitation: blood, plasma, saliva or urine), a tissue sample from a subject at risk of having or developing breast cancer (e.g. a biopsy such as a breast biopsy) or homogenate thereof. The methods and compositions of the present invention are useful for screening and diagnosis of a living subject, but may also be used for post-mortem diagnosis in a subject, for example, to identify if family members of the subject would be at risk of developing the same disease.

The following definitions are provided to assist in the review of the instant disclosure.

Definitions

“Feature” refers to a spot identified in a 2D gel, and the term “Breast Cancer—Associated Feature” (BF) refers to a feature that is differentially present in a first sample or sample set from a subject having breast cancer compared with a second sample or sample set from a subject free from breast cancer. A feature or spot identified in a 2D gel is characterised by its isoelectric point (pI) and apparent molecular weight (MW) as determined by 2D gel electrophoresis, particularly utilising the Preferred Technology described herein. As used herein, a feature is “differentially present” in a first sample or sample set with respect to a second sample or sample set when a method for detecting the said feature (e.g., 2D electrophoresis) gives a different signal when applied to the first and second samples or sample sets. A BF, (or a Protein Isoform, i.e. BPI, as defined infra) is “increased” in the first sample or sample set with respect to the second sample or sample set if the method of detection indicates that the BF, or BPI is more abundant in the first sample or sample set than in the second sample or sample set, or if the BF, or BPI is detectable in the first sample or sample set and substantially undetectable in the second sample or sample set. Conversely, a BF, or BPI is “decreased” in the first sample or sample set with respect to the second sample or sample set if the method of detection indicates that the BF, or BPI is less abundant in the first sample or sample set than in the second sample or sample set or if the BF, or BPI is undetectable in the first sample or sample set and detectable in the second sample or sample set.

Particularly, the relative abundance of a feature in the two samples or sample sets is determined in reference to its normalised signal, in two steps. First, the signal obtained upon detecting the feature in a first sample or sample set is normalised by reference to a suitable background parameter, e.g., (a) to the total protein in the sample being analysed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed in Table III, or (c) more preferably to the total signal detected as the sum of each of all proteins in the sample.

Secondly, the normalised signal for the feature in the first sample or sample set is compared with the normalised signal for the same feature in the second sample or sample set in order to identify features that are “differentially present” in the first sample or sample set with respect to the second sample or sample set.

“Fold change” includes “fold increase” and “fold decrease” and refers to the relative increase or decrease in abundance of a BF or the relative increase or decrease in expression or activity of a polypeptide (e.g. a BPI, as defined infra.) in a first sample or sample set compared to a second sample or sample set. A BF or polypeptide fold change may be measured by any technique known to those of skill in the art, albeit the observed increase or decrease will vary depending upon the technique used. Preferably, fold change is determined herein as described in the Examples infra.

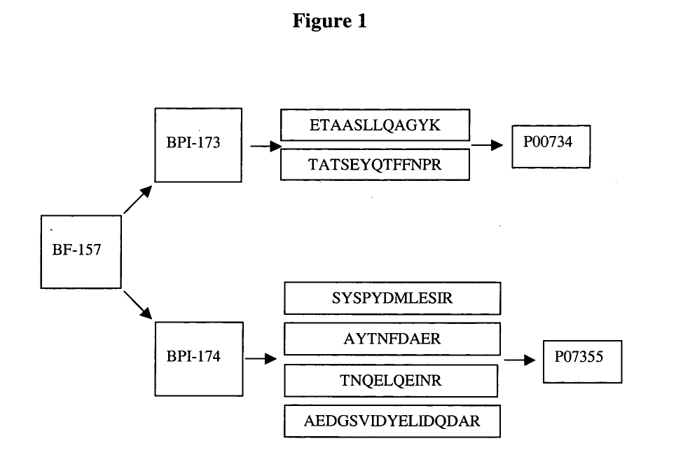

“Breast Cancer-Associated Protein Isoform” (BPI) refers to a polypeptide that is differentially present in a first sample or sample set from a subject having breast cancer compared with a second sample or sample set from a subject free from breast cancer. As used herein, a BPI is “differentially present” in a first sample or sample set with respect to a second sample or sample set when a method for detecting the said feature, (e.g., 2D electrophoresis or immunoassay) gives a different signal when applied to the first and second samples or sample sets (as described above in relation to BFs). A BPI is characterised by one or more peptide sequences of which it is comprised, and further by a pI and MW, preferably determined by 2D electrophoresis, particularly utilising the Preferred Technology as described herein. Typically, BPIs are identified or characterised by the amino acid sequencing of BFs (FIG. 1).

A BPI is characterised as, or by, a particular peptide sequence associated with its pI and MW. As depicted herein, a BF may comprise one or more BPI(s), which have indistinguishable pIs and MWs using the Preferred Technology, but which have distinct peptide sequences. The peptide sequence(s) of the BPI can be utilised to search database(s) for previously identified proteins comprising such peptide sequence(s). In some instances, it can be ascertained whether a commercially available antibody exists which may recognise the previously identified protein and/or a variant thereof. Preferably the BPI corresponds to the previously identified protein, or be a variant of the previously identified protein.

“Variant” as used herein refers to a polypeptide which is a member of a family of polypeptides that are encoded by a single gene or from a gene sequence within a family of related genes and which differ in their pI or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

As used herein, the terms “MW” and “pI” are defined, respectively, to mean the apparent molecular weight and the apparent isoelectric point of a feature or protein isoform as measured in exact accordance with the Reference Protocol identified in Section 6 below.

“Modulate” in reference to expression or activity of a BF, BPI or BPI-related polypeptide refers to any change, e.g., upregulation or downregulation, increase or decrease, of the expression or activity of the BF, BPI or BPI-related polypeptide. Those skilled in the art, based on the present disclosure, will understand that such modulation can be determined by assays known to those of skill in the art.

As used herein, an “aberrant level” means a level that is increased or decreased in a first sample compared with the level in a second sample from a subject free from breast cancer or a reference level.

“Cluster” refers to a group of BFs (or their associated BPIs) identified by multivariate statistical analysis on the proteome of cells isolated from subjects with no, primary or metastasised breast cancer disease. Preferably a cluster contains at least 2 BF (or BPIs), or at least 5 BFs (or BPIs).

“BPI analog” refers to a polypeptide that possesses similar or identical function(s) as a BPI but need not necessarily comprise an amino acid sequence that is similar or identical to the amino acid sequence of the BPI, or possess a structure that is similar or identical to that of the BPI. As used herein, an amino acid sequence of a polypeptide is “similar” to that of a BPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the BPI; (b) the polypeptide is encoded by a nucleotide sequence that hybridises under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the BPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the BPI. As used herein, a polypeptide with “similar structure” to that of a BPI refers to a polypeptide that has a similar secondary, tertiary or quarternary structure as that of the BPI. The structure of a polypeptide can determined by methods known to those skilled in the art, including but not limited to, X-ray crystallography, nuclear magnetic resonance, and crystallographic electron microscopy.

“BPI fusion protein” refers to a polypeptide that comprises (i) an amino acid sequence of a BPI, BPI fragment, BPI-related polypeptide or a fragment of a BPI-related polypeptide and (ii) an amino acid sequence of a heterologous polypeptide (i.e., a non-BPI, non-BPI fragment or non-BPI-related polypeptide).

“BPI homolog” refers to a polypeptide that comprises an amino acid sequence similar to that of a BPI but does not necessarily possess a similar or identical function as the BPI.

“BPI ortholog” refers to a non-human polypeptide that (i) comprises an amino acid sequence similar to that of a BPI and (ii) possesses a similar or identical function to that of the BPI.

“BPI-related polypeptide” refers to a BPI homolog, a BPI analog, a variant of a BPI, a BPI ortholog, a fragment thereof, or any combination thereof.

“Chimeric Antibody” refers to a molecule in which different portions are derived from different animal species, such as those having a human immunoglobulin constant region and a variable region derived from a murine mAb. (See, e.g., U.S. Pat. No. 4,816,567; and U.S. Pat. No. 4,816,397). For example, a portion of the antibody may be fused with the constant domain of immunoglobulins (IgA, IgE, IgG, IgM), or portions thereof (CH1, CH2, CH3, or any combination thereof and portions thereof) resulting in chimeric antibodies.

“Humanised Antibody” refers to a molecule from non-human species having one or more complementary determining regions (CDRs) from the non-human species and a framework region from a human immunoglobulin molecule.

“Derivative” refers to a polypeptide that comprises an amino acid sequence of a second polypeptide that has been altered by the introduction of at least one amino acid residue substitution, deletion or addition. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. Preferably the fragment of a BPI possesses the functional activity of the BPI.

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al., (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilised as described in Altschul et al., (1997) Nucleic Acids Res. 25:3389-3402. Alternatively, PSI-Blast can be used to perform an iterated search which detects distant relationships between molecules (Id.). When utilising BLAST, Gapped BLAST, and PSI-BLAST programs, the default parameters of the respective programs (e.g., XBLAST and NBLAST) can be used.

Another example of a mathematical algorithm utilised for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

“Diagnosis” refers to diagnosis, prognosis, monitoring, characterising, selecting patients, including participants in clinical trials, and identifying patients at risk for or having a particular disorder or those most likely to respond to a particular therapeutic treatment, or for assessing or monitoring a patient's response to a particular therapeutic treatment.

“Treatment” refers to therapy, prevention and prophylaxis and particularly refers to the administration of medicine or the performance of medical procedures with respect to a patient, for either prophylaxis (prevention) or to cure the infirmity or malady in the instance where the patient is afflicted.

“Agent” refers to all materials that may be used to prepare pharmaceutical and diagnostic compositions, or that may be compounds, agonists, antagonists, nucleic acids, polypeptides, fragments, isoforms, variants, or other materials that may be used independently for such purposes, all in accordance with the present invention.

“Highly stringent conditions” refers to hybridisation to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & Sons, Inc., New York, at p. 2.10.3)

For some applications, less stringent conditions for duplex formation are required. As used herein “moderately stringent conditions” refers to washing in 0.2×SSC/0.1% SDS at 42° C. (Ausubel et al., 1989, supra).

As used herein, a BPI is “isolated” when it is present in a preparation that is substantially free of contaminating proteins, i.e., a preparation in which less than 10% (preferably less than 5%, more preferably less than 1%) of the total protein present is contaminating protein(s)

As used herein, a “biological sample” can be obtained from any source, including a body fluid sample such as serum, blood, plasma, urine or a tissue sample, e.g. breast tissue sample.

“Serum” refers to the supernatant fluid produced by clotting and centrifugal sedimentation of a blood sample.

“Plasma” refers to the supernatant fluid produced by inhibition of clotting (for example, by citrate or EDTA) and centrifugal sedimentation of a blood sample.

“Blood” as used herein includes serum and plasma.

“Two-dimensional electrophoresis” (2D-electrophoresis) means a technique comprising denaturing electrophoresis, followed by isoelectric focusing; this generates a two-dimensional gel (2D-gel) containing a plurality of separated proteins.

“Breast tissue” refers to cells derived from breast tissue from the breast itself, as well as the tissue adjacent to and/or within the strata underlying the breast.

“Breast cancer sub-type” as used herein refers to one of the following types of breast cancer: primary breast cancer or metastatic breast cancer.

The “Preferred Technology”

Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in WO 98/23950 and in U.S. Pat. Nos. 6,064,654, and 6,278,794, with particular reference to the protocol of WO 98/23950. Briefly, the Preferred Technology provides efficient, computer-assisted methods and apparatus for identifying, selecting and characterising biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labelled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is a modified version of the above described scanner. In the preferred scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the preferred scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the preferred scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the preferred scanner have been inverted. In the preferred scanner, the laser, mirror, waveguide and other optical components are above the glass plate being scanned. The scanner described in the Basiji thesis has these components underneath. In the preferred scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is a modified version of the preferred scanner, in which the signal output is digitised to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

Breast Cancer-Associated Features (BFs)

In one aspect of the invention, two-dimensional electrophoresis is used to analyse serum from a subject, preferably a living subject, in order to detect or quantify the expression of one or more Breast Cancer-Associated Features (BFs) or a cluster of BFs for screening, prevention or diagnosis of breast cancer, to determine the prognosis of a subject having breast cancer, to monitor progression of breast cancer, to monitor the effectiveness of breast cancer therapy, for identifying patients most likely to respond to a particular therapeutic treatment, or for drug development.

By way of example and not of limitation, using the Preferred Technology, a number of samples from subjects having breast cancer and samples from subjects free from breast cancer are separated by two-dimensional electrophoresis, and the fluorescent digital images of the resulting gels are matched to a chosen representative primary master gel image. This process allows any gel feature, characterised by its pI and MW, to be identified and examined on any gel of the study. In particular, the amount of protein present in a given feature can be measured in each gel; this feature abundance can be averaged amongst gels from similar samples (e.g. gels from samples from subjects having primary breast cancer or gels from patients having metastatic breast cancer). Finally, statistical analyses can be conducted on the thus created sample sets, in order to compare 2 or more sample sets to each other.

The BFs disclosed herein have been identified by comparing serum samples from subjects having primary breast cancer or serum samples from patients with metastatic breast cancer against serum samples from subjects free from breast cancer. Subjects free from breast cancer include subjects with no known disease or condition (normal subjects) and subjects with diseases (including mammary pathologies) other than breast cancer. BFs have been identified through the methods and apparatus of the Preferred Technology that are decreased or increased in the serum of subjects having breast cancer as compared with the serum of subjects free from breast cancer. These BFs can be described by apparent molecular weight (MW) and isoelectric point (pI) as provided in Table I.

One skilled in the art may analyse a master gel image (as described above) to obtain a cluster of BFs, from those listed in Table I, that can discriminate amongst, primary breast cancer, metastatic breast cancer and control subjects. A sample from a subject can be analysed for the levels present of the BFs comprising a cluster in order to determine whether the subject has primary breast cancer, metastatic breast cancer or is free from breast cancer. BFs which show particular utility in a cluster are given in Cluster I and Cluster II. Thus, the levels present of BFs comprising the cluster in a sample from a subject can be used to diagnose breast cancer and the sub-type of breast cancer.

TABLE I
BFs Identified in Serum of Subjects With Breast Cancer

| Feature (BF) | pI | MW (Da) |
|---|---|---|
| BF-101 | 5.06 | 192,161 |
| BF-102 | 6.24 | 191,412 |
| BF-103 | 5.56 | 153,505 |
| BF-104 | 6.17 | 121,435 |
| BF-105 | 7.22 | 109,113 |
| BF-106 | 4.73 | 87,712 |
| BF-107 | 7.44 | 86,906 |
| BF-108 | 6.08 | 78,042 |
| BF-109 | 6.02 | 59,414 |
| BF-110 | 4.90 | 55,348 |
| BF-111 | 6.55 | 53,549 |
| BF-112 | 5.32 | 48,561 |
| BF-113 | 6.90 | 48,630 |
| BF-114 | 4.70 | 48,182 |
| BF-115 | 7.48 | 48,169 |
| BF-116 | 5.51 | 45,867 |
| BF-117 | 5.08 | 43,858 |
| BF-118 | 5.02 | 43,942 |
| BF-119 | 6.46 | 43,553 |
| BF-120 | 4.76 | 43,563 |
| BF-121 | 5.95 | 36,016 |
| BF-122 | 4.61 | 35,101 |
| BF-123 | 4.56 | 29,031 |
| BF-124 | 6.84 | 27,312 |
| BF-125 | 4.71 | 26,996 |
| BF-126 | 5.72 | 27,034 |
| BF-127 | 6.89 | 23,542 |
| BF-128 | 5.67 | 22,027 |
| BF-129 | 6.22 | 122,600 |
| BF-130 | 4.61 | 81,856 |
| BF-131 | 5.75 | 69,522 |
| BF-132 | 5.20 | 61,296 |
| BF-133 | 5.75 | 58,171 |
| BF-134 | 5.53 | 31,784 |
| BF-135 | 5.23 | 22,982 |
| BF-136 | 7.06 | 15,240 |
| BF-137 | 5.86 | 196,396 |
| BF-138 | 5.46 | 187,242 |
| BF-139 | 7.49 | 119,667 |
| BF-140 | 6.16 | 100,014 |
| BF-141 | 5.63 | 91,596 |
| BF-142 | 5.15 | 86,344 |
| BF-143 | 4.78 | 86,825 |
| BF-144 | 4.56 | 66,507 |
| BF-145 | 5.15 | 63,880 |
| BF-146 | 5.32 | 63,088 |
| BF-147 | 5.03 | 64,235 |
| BF-148 | 4.98 | 62,149 |
| BF-149 | 4.53 | 59,743 |
| BF-150 | 5.52 | 53,454 |
| BF-151 | 4.55 | 50,129 |
| BF-152 | 4.10 | 44,095 |
| BF-153 | 4.71 | 43,530 |
| BF-155 | 6.14 | 43,163 |
| BF-156 | 6.88 | 39,900 |
| BF-157 | 7.66 | 34,359 |
| BF-158 | 6.28 | 32,635 |
| BF-159 | 5.65 | 31,545 |
| BF-160 | 6.74 | 27,296 |
| BF-161 | 5.15 | 25,593 |
| BF-162 | 6.73 | 24,401 |
| BF-163 | 7.82 | 23,857 |
| BF-164 | 5.52 | 22,177 |
| BF-165 | 6.00 | 15,113 |
| BF-166 | 8.73 | 12,102 |
| BF-509 | 5.5 | 57,934 |
| BF-510 | 4.5 | 51,499 |
| BF-511 | 4.6 | 44,998 |
| BF-512 | 5.6 | 92,686 |
| BF-513 | 6.0 | 79,323 |
| BF-514 | 5.1 | 65,901 |
| BF-515 | 5.1 | 63,379 |
| BF-516 | 5.2 | 61,951 |
| BF-517 | 4.9 | 61,074 |
| BF-518 | 5.3 | 60,714 |
| BF-519 | 5.3 | 57,026 |
| BF-519 | 5.3 | 57,026 |
| BF-520 | 5.0 | 46,200 |

Specific clusters of BFs and BPIs that can be used to determine the breast cancer stage are provided in the examples (section 6.3).

For any given BF, the signal obtained upon analysing a biological sample or sample set from subjects having breast cancer relative to the signal obtained upon analysing a biological sample or sample set from subjects free from breast cancer will depend upon the particular analytical protocol and detection technique that is used. Accordingly, the present invention contemplates that each laboratory will, based on the present description, establish a reference range for each BF in subjects free from breast cancer according to the analytical protocol and detection technique in use, as is conventional in the diagnostic art. Preferably, at least one positive biological control sample or sample set from a subject known to have breast cancer or at least one negative control biological sample or sample set from a subject known to be free from breast cancer (and more preferably both positive and negative control samples) are included in each batch of test samples analysed. In one embodiment, the level of expression of a feature is determined relative to a background value, which is defined as the level of signal obtained from a proximal region of the image that (a) is equivalent in area to the particular feature in question; and (b) contains no discernible protein feature.

In a preferred embodiment, the signal associated with a BF in the serum of a subject (e.g., a subject suspected of having or known to have breast cancer is normalised with reference to one or more ERFs detected in the same 2D gel. As will be apparent to one of ordinary skill in the art, such ERFs may readily be determined by comparing different samples using the Preferred Technology. Suitable ERFs include (but are not limited to) those described in Table II.

TABLE II
Expression Reference Features

| ERF# | MW (Da) | pI |
|---|---|---|
| ERF-1 | 53370 | 6.17 |
| ERF-2 | 30780 | 5.03 |

As those of skill in the art will readily appreciate, the apparent MW and pI of a given feature or protein isoform will vary to some extent depending on the precise protocol used for each step of the 2D electrophoresis and for landmark matching (as described in section 6.1.9 infra). When the Reference Protocol is followed and when samples are run in duplicate or a higher number of replicates, variation in the measured mean pI of a BF or BPI is typically less than 3% and variation in the measured mean MW of a BF or BPI is typically less than 5%. Where the skilled artisan wishes to deviate from the Reference Protocol, calibration experiments should be performed to compare the MW and pI for each BF or BPI as detected (a) by the Reference Protocol and (b) by the deviant protocol.

Four subsets of BFs were identified as follows:

List 1: BFs decreased in serum from subjects with primary breast cancer: BF-103, BF-106, BF-111, BF-112, BF-114, BF-115, BF-117, BF-118, BF-119, BF-122, BF-126, BF-127, BF-128, BF-130, BF-131, BF-132, BF-134 and BF-135.

List 2: BFs increased in serum from subjects with primary breast cancer: BF-101, BF-102, BF-104, BF-105, BF-107, BF-108, BF-109, BF-10, BF-113, BF-116, BF-120, BF-121, BF-123, BF-124, BF-125, BF-129, BF-133 and BF-136.

List 3: BFs decreased in serum from subjects with metastatic breast cancer: BF-130, BF-131, BF-132, BF-134, BF-135, BF-137, BF-139, BF-142, BF-143, BF-145, BF-146, BF-147, BF-148, BF-149, BF-150, BF-155, BF-157, BF-158, BF-159, BF-160, BF-162, BF-164, BF-165 and BF-166.

List 4: BFs increased in serum from subjects with metastatic breast cancer: BF-129, BF-133, BF-136, BF-138, BF-140, BF-141, BF-144, BF-151, BF-152, BF-153, BF-156, BF-161 and BF-163.

Two clusters of BFs were identified:

Cluster I: BF-108, BF-132, BF-141, BF-147, BF-512, BF-513, BF-514, BF-515, BF-516, BF-517, BF-518, BF-519, BF-520

Cluster II: BF-132, BF-151, BF-157, BF-509, BF-510, BF-511

BFs can be used for detection, diagnosis, or monitoring of breast cancer, or for identifying patients most likely to respond to a specific therapeutic treatment, or for drug development. In one embodiment of the invention, a first biological sample or sample set from a subject (e.g., a subject suspected of having breast cancer) is analysed by 2D electrophoresis for quantitative detection of one or more of the BFs as defined in List 1. A decreased abundance of said one or more of these BFs in the first sample from the subject relative to a second sample from a subject or subjects free from breast cancer (e.g., a control sample or a previously determined reference range) indicates the presence of primary breast cancer.

In another embodiment of the invention, a first biological sample from a subject is analysed by 2D electrophoresis for the quantitative detection of one or more of the BFs as defined in List 2. An increased abundance of said one or more BFs in the first sample from the subject relative to a second sample from a subject or subjects free from breast cancer (e.g., a control sample or a previously determined reference range) indicates the presence of primary breast cancer.

In yet another embodiment, a first biological sample from a subject is analysed by 2D electrophoresis for quantitative detection of (a) one or more BFs or any combination of them, whose decreased abundance indicates the presence of primary breast cancer, i.e., BFs as defined in List 1; and (b) one or more BFs or any combination of them, whose increased abundance indicates the presence of primary breast cancer i.e., BFs as defined in List 2.

In yet another embodiment of the invention, a first biological sample from a subject is analysed by 2D electrophoresis for quantitative detection of one or more of the BFs as defined in Lists 1 and 2; wherein the ratio of the one or more BFs relative to an Expression Reference Feature (ERF) indicates whether primary breast cancer is present. In a specific embodiment, a decrease in one or more BF/ERF ratios in a first sample relative to the BF/ERF ratios in a second sample or a reference range indicates the presence of primary breast cancer; i.e. the BFs as defined in List 1 are suitable for this purpose. In another specific embodiment, an increase in one or more BF/ERF ratios in a first sample relative to the BF/ERF ratios in a second sample or a reference range indicates the presence of primary breast cancer; the BFs as defined in List 2 are suitable BFs for this purpose.

In a further embodiment of the invention, a first biological sample from a subject is analysed by 2D electrophoresis for quantitative detection of (a) one or more BFs, or any combination of them, whose decreased BF/ERF ratio(s) in a first sample relative to the BF/ERF ratio(s) in a second sample indicates the presence of primary breast cancer, i.e., the BFs as defined in List 1; and (b) one or more BFs, or any combination of them, whose increased BF/ERF ratio(s) in a first sample relative to the BF/ERF ratio(s) in a second sample indicates the presence of primary breast cancer, i.e., the BFs as defined in List 2.

In yet another embodiment of the invention, a first biological sample from a subject (e.g., a subject suspected of having breast cancer) is analysed by 2D electrophoresis for quantitative detection of one or more of the BFs as defined in List 3. A decreased abundance of said one or more of these BFs in the first sample from the subject relative to a second sample from a subject or subjects free from breast cancer (e.g., a control sample or a previously determined reference range) indicates the presence of metastatic breast cancer.

In yet another embodiment of the invention, a first biological sample from a subject is analysed by 2D electrophoresis for the quantitative detection of one or more of the BFs as defined in List 4. An increased abundance of said one or more BFs in the first sample from the subject relative to a second sample from a subject or subjects free from breast cancer (e.g., a control sample or a previously determined reference range) indicates the presence of metastatic breast cancer.

In yet another embodiment, a first biological sample from a subject is analysed by 2D electrophoresis for quantitative detection of (a) one or more BFs or any combination of them, whose decreased abundance indicates the presence of metastatic breast cancer, i.e., BFs as defined in List 3; and (b) one or more BFs or any combination of them, whose increased abundance indicates the presence of metastatic breast cancer i.e., BFs as defined in List 4.

In yet another embodiment of the invention, a first biological sample from a subject is analysed by 2D electrophoresis for quantitative detection of one or more of the BFs as defined in Lists 3 and 4; wherein the ratio of the one or more BFs relative to an Expression Reference Feature (ERF) indicates whether metastatic breast cancer is present. In a specific embodiment, a decrease in one or more BF/ERF ratios in a first sample relative to the BF/ERF ratios in a second sample or a reference range indicates the presence of metastatic breast cancer; i.e. the BFs as defined in List 3 are suitable for this purpose. In another specific embodiment, an increase in one or more BF/ERF ratios in a first sample relative to the BF/ERF ratios in a second sample or a reference range indicates the presence of metastatic breast cancer; the BFs as defined in List 4 are suitable BFs for this purpose.

In a further embodiment of the invention, a first biological sample from a subject is analysed by 2D electrophoresis for quantitative detection of (a) one or more BFs, or any combination of them, whose decreased BF/ERF ratio(s) in a first sample relative to the BF/ERF ratio(s) in a second sample indicates the presence of metastatic breast cancer, i.e., the BFs as defined in List 3; and (b) one or more BFs, or any combination of them, whose increased BF/ERF ratio(s) in a first sample relative to the BF/ERF ratio(s) in a second sample indicates the presence of metastatic breast cancer, i.e., the BFs as defined in List 4.

In a preferred embodiment, a biological sample from a subject is analysed for quantitative detection of a plurality of BFs. In a more preferred embodiment, a biological sample from a subject is analysed for the quantitative detection of a cluster of BFs as described in Cluster I or Cluster II, wherein an altered abundance of the BFs within the cluster distinguishes between patients with primary breast cancer, metastatic breast cancer and control subjects.

Breast Cancer-Associated Protein Isoforms (BPIs)

In another aspect of the invention, a biological sample from a subject, preferably a living subject, is analysed for quantitative detection of one or more Breast Cancer-Associated Protein Isoforms (BPIs) for screening or diagnosis of breast cancer, to monitor the effectiveness of breast cancer therapy, for identifying patients most likely to respond to a particular therapeutic treatment or for drug development. As is well known in the art, a given protein may be expressed as variants that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. It follows that differential presence of a protein isoform does not require differential expression of the gene encoding the protein in question. As used herein, the term “Breast Cancer-Associated Protein Isoform” refers to a polypeptide that is differentially present in a first biological sample from a subject having breast cancer compared with second sample from a subject free from breast cancer.

BPIs are described herein by the amino acid sequencing of BFs. BPIs were isolated, subjected to proteolysis, and analysed by mass spectrometry using the methods and apparatus of the Preferred Technology. One skilled in the art can identify sequence information from proteins analysed by mass spectrometry and/or tandem mass spectrometry using various spectral interpretation methods and database searching tools. Examples of some of these methods and tools can be found at the Swiss Institute of Bioinformatics web site at http://www.expasy.con/, and the European Molecular Biology Laboratory web site at http://www.narrador.embl-heidelberg.de/GroupPages/PageLink/peptidesearchpage.html. Identification of BPIs was performed primarily using the SEQUEST search program (Eng et al., 1994, J. Am. Soc. Mass Spectrom. 5:976-989) and the method described in WO 02/21139.

The BPIs are comprised of those that are decreased or increased in the serum of subjects having breast cancer as compared with the serum of subjects free from breast cancer. The amino acid sequences of peptides produced from these BPIs by proteolysis using trypsin and identified by tandem mass spectrometry and database searching as described in the Examples, infra are listed in Table III, in addition to their corresponding pIs and MWs.

TABLE III
BPIs Identified in Serum of Subjects having Breast Cancer

| Feature | Isoform | | | Amino Acid Sequences of | |
|---|---|---|---|---|---|
| (BF) | (BPI) | pI | MW (Da) | Tryptic Digest Peptides | SEQ ID NO |
| BF-104 | BPI-186 | 6.17 | 121,435 | ALNHLPLEYNSALYSR | SEQ ID NO: 10 |
| | | | | QLEWGLER | SEQ ID NO: 97 |
| | | | | GFVVAGPSR | SEQ ID NO: 56 |
| BF-105 | BPI-101 | 7.22 | 109,113 | FVTWIEGVMR | SEQ ID NO: 53 |
| | | | | YEFLNGR | SEQ ID NO: 147 |
| | | | | HSIFTPETNPR | SEQ ID NO: 62 |
| BF-107 | BPI-187 | 7.44 | 86,906 | TIYTPGSTVLYR | SEQ ID NO: 122 |
| | | | | IPIEDGSGEVVLSR | SEQ ID NO: 67 |
| BF-108 | BPI-102 | 6.08 | 78,042 | VSVFVPPR | SEQ ID NO: 137 |
| | | | | DGFFGNPR | SEQ ID NO: 22 |
| BF-108 | BPI-103 | 6.08 | 78,042 | ETAASLLQAGYK | SEQ ID NO: 40 |
| BF-108 | BPI-104 | 6.08 | 78,042 | RVWELSK | SEQ ID NO: 106 |
| | | | | EQLQDMGLVDLFSPEK | SEQ ID NO: 38 |
| | | | | EVPLNTIIFMGR | SEQ ID NO: 43 |
| | | | | LPGIVAEGR | SEQ ID NO: 82 |
| | | | | DDLYVSDAFHK | SEQ ID NO: 20 |
| BF-109 | BPI-188 | 6.02 | 59,414 | DTGTYGFLLPER | SEQ ID NO: 27 |
| BF-110 | BPI-111 | 4.90 | 55,348 | ISEQFTAMFR | SEQ ID NO: 69 |
| | | | | FPGQLNADLR | SEQ ID NO: 48 |
| BF-112 | BPI-113 | 5.32 | 48,561 | DYLLLVMEGTDDGR | SEQ ID NO: 28 |
| BF-114 | BPI-114 | 4.70 | 48,182 | VLSLAQEQVGGSPEK | SEQ ID NO: 134 |
| | | | | TEQWSTLPPETK | SEQ ID NO: 119 |
| | | | | AEMADQAAAWLTR | SEQ ID NO: 5 |
| BF-115 | BPI-115 | 7.48 | 48,169 | IVQLIQDTR | SEQ ID NO: 72 |
| | | | | FPPEEELQR | SEQ ID NO: 49 |
| | | | | SIPQVSPVR | SEQ ID NO: 112 |
| BF-116 | BPI-117 | 5.51 | 45,867 | YENEVALR | SEQ ID NO: 148 |
| | | | | ELTTEIDNNIEQISSYK | SEQ ID NO: 34 |
| BF-117 | BPI-118 | 5.08 | 43,858 | TQVNTQAEQLR | SEQ ID NO: 125 |
| | | | | ALVQQMEQLR | SEQ ID NO: 12 |
| | | | | LEPYADQLR | SEQ ID NO: 78 |
| BF-118 | BPI-191 | 5.02 | 43,942 | SLAELGGHLDQQVEEFR | SEQ ID NO: 113 |
| | | | | ALVQQMEQLR | SEQ ID NO: 12 |
| | | | | RVEPYGENFNK | SEQ ID NO: 105 |
| BF-119 | BPI-119 | 6.46 | 43,553 | FPPEEELQR | SEQ ID NO: 49 |
| | | | | SIPQVSPVR | SEQ ID NO: 112 |
| BF-120 | BPI-120 | 4.76 | 43,563 | VYAYYNLEESCTR | SEQ ID NO: 141 |
| | | | | VHQYFNVELIQPGAVK | SEQ ID NO: 132 |
| | | | | SGSDEVQVGQQR | SEQ ID NO: 111 |
| | | | | GQGTLSVVTMYHAK | SEQ ID NO: 58 |
| BF-122 | BPI-121 | 4.61 | 35,101 | ASSIIDELFQDR | SEQ ID NO: 14 |
| BF-123 | BPI-123 | 4.56 | 29,031 | NILTSNNIDVK | SEQ ID NO: 90 |
| | | | | IPTTFENGR | SEQ ID NO: 68 |
| BF-124 | BPI-124 | 6.84 | 27,312 | SNLDEDIIAEENIVSR | SEQ ID NO: 115 |
| | | | | NEQVEIR | SEQ ID NO: 88 |
| | | | | AVLYNYR | SEQ ID NO: 17 |
| BF-126 | BPI-125 | 5.72 | 27,034 | YVLTQPPSVSVAPGQTAR | SEQ ID NO: 155 |
| | | | | FSGSNSGNTATLTISR | SEQ ID NO: 51 |
| | | | | AAPSVTLFPPSSEELQANK | SEQ ID NO: 2 |
| BF-127 | BPI-126 | 6.89 | 23,542 | YAASSYLSLTPEQWK | SEQ ID NO: 146 |
| | | | | TVAAPSVFIFPPSDEQLK | SEQ ID NO: 126 |
| | | | | LLIYGASSR | SEQ ID NO: 81 |
| BF-127 | BPI-127 | 6.89 | 23,542 | EIVLTQSPGTLSLSPGER | SEQ ID NO: 33 |
| BF-128 | BPI-189 | 5.67 | 22,027 | FLVGPDGIPIMR | SEQ ID NO: 47 |
| | | | | QEPGENSEILPTLK | SEQ ID NO: 94 |
| BF-129 | BPI-192 | 6.22 | 122,600 | SEYGAALAWEK | SEQ ID NO: 109 |
| | | | | DLHLSDVFLK | SEQ ID NO: 24 |
| | | | | GFVVAGPSR | SEQ ID NO: 56 |
| BF-130 | BPI-128 | 4.61 | 81,856 | EIGELYLPK | SEQ ID NO: 31 |
| BF-131 | BPI-129 | 5.75 | 69,522 | VWVYPPEK | SEQ ID NO: 140 |
| | | | | SGAQATWTELPWPHEK | SEQ ID NO: 110 |
| | | | | RLWWLDLK | SEQ ID NO: 101 |
| | | | | EWFWDLATGTMK | SEQ ID NO: 45 |
| BF-132 | BPI-130 | 5.20 | 61,296 | VAEGTQVLELPFK | SEQ ID NO: 128 |
| | | | | RVWELSK | SEQ ID NO: 106 |
| | | | | LPGIVAEGR | SEQ ID NO: 82 |
| | | | | GDDITMVLILPKPEK | SEQ ID NO: 54 |
| | | | | EVPLNTIIFMGR | SEQ ID NO: 43 |
| | | | | EQLQDMGLVDLFSPEK | SEQ ID NO: 38 |
| BF-132 | BPI-131 | 5.20 | 61,296 | SPEQQETVLDGNLIIR | SEQ ID NO: 116 |
| BF-133 | BPI-133 | 5.75 | 58,171 | ATVVYQGER | SEQ ID NO: 15 |
| | | | | KATVVYQGER | SEQ ID NO: 75 |
| | | | | WLQGSQELPR | SEQ ID NO: 144 |
| BF-133 | BPI-135 | 5.75 | 58,171 | YLTWASR | SEQ ID NO: 153 |
| BF-134 | BPI-138 | 5.53 | 31,784 | AADDTWEPFASGK | SEQ ID NO: 1 |
| | | | | GSPAINVAVHVFR | SEQ ID NO: 59 |
| | | | | KAADDTWEPFASGK | SEQ ID NO: 74 |
| BF-135 | BPI-139 | 5.23 | 22,982 | VQPYLDDFQK | SEQ ID NO: 135 |
| | | | | LHELQEK | SEQ ID NO: 79 |
| | | | | EQLGPVTQEFWDNLEK | SEQ ID NO: 37 |
| | | | | WQEEMELYR | SEQ ID NO: 145 |
| | | | | DEPPQSPWDR | SEQ ID NO: 21 |
| | | | | AKPALEDLR | SEQ ID NO: 8 |
| | | | | THLAPYSDELR | SEQ ID NO: 120 |
| BF-137 | BPI-143 | 5.86 | 196,396 | YVTSAPMPEPQAPGR | SEQ ID NO: 156 |
| | | | | QIQVSWLR | SEQ ID NO: 96 |
| BF-137 | BPI-144 | 5.86 | 196,396 | LPPNVVEESAR | SEQ ID NO: 83 |
| | | | | LVHVEEPHTETVR | SEQ ID NO: 86 |
| | | | | HYDGSYSTFGER | SEQ ID NO: 64 |
| | | | | AIGYLNTGYQR | SEQ ID NO: 7 |
| | | | | VGFYESDVMGR | SEQ ID NO: 131 |
| BF-138 | BPI-145 | 5.46 | 187,242 | EFDHNSNIR | SEQ ID NO: 29 |
| | | | | RPYFPVAVGK | SEQ ID NO: 102 |
| | | | | NGFYPATR | SEQ ID NO: 89 |
| | | | | IDVHLVPDR | SEQ ID NO: 65 |
| | | | | EIMENYNIALR | SEQ ID NO: 32 |
| BF-139 | BPI-146 | 7.49 | 119,667 | TLLPVSKPEIR | SEQ ID NO: 123 |
| | | | | GIYGTISR | SEQ ID NO: 57 |
| | | | | EGMLSIMSYR | SEQ ID NO: 30 |
| | | | | AFTECCVVASQLR | SEQ ID NO: 6 |
| | | | | ESYSGVTLDPR | SEQ ID NO: 39 |
| | | | | LQGTLPVEAR | SEQ ID NO: 84 |
| BF-140 | BPI-147 | 6.16 | 100,014 | VLFYVDSEK | SEQ ID NO: 133 |
| | | | | DGFVQDEGTMFPVGK | SEQ ID NO: 23 |
| BF-140 | BPI-148 | 6.16 | 100,014 | VASYGVKPR | SEQ ID NO: 129 |
| | | | | QLNEINYEDHK | SEQ ID NO: 98 |
| | | | | ISVIRPSK | SEQ ID NO: 70 |
| BF-141 | BPI-149 | 5.63 | 91,596 | RVWELSK | SEQ ID NO: 106 |
| | | | | VAEGTQVLELPFK | SEQ ID NO: 128 |
| | | | | LPGIVAEGR | SEQ ID NO: 82 |
| | | | | DDLYVSDAFHK | SEQ ID NO: 20 |
| BF-141 | BPI-150 | 5.63 | 91,596 | ETAASLLQAGYK | SEQ ID NO: 40 |
| BF-142 | BPI-152 | 5.15 | 86,344 | FTFEYSR | SEQ ID NO: 52 |
| BF-143 | BPI-153 | 4.78 | 86,825 | VRPQQLVK | SEQ ID NO: 136 |
| | | | | SPEQQETVLDGNLIIR | SEQ ID NO: 116 |
| | | | | LALDNGGLAR | SEQ ID NO: 77 |
| | | | | FAHTVVTSR | SEQ ID NO: 46 |
| BF-144 | BPI-154 | 4.56 | 66,507 | WEMPFDPQDTHQSR | SEQ ID NO: 142 |
| | | | | AVLDVFEEGTEASAATAVK | SEQ ID NO: 16 |
| | | | | LYGSEAFATDFQDSAAAK | SEQ ID NO: 87 |
| | | | | ITLLSALVETR | SEQ ID NO: 71 |
| | | | | ADLSGITGAR | SEQ ID NO: 3 |
| BF-145 | BPI-155 | 5.15 | 63,880 | WLQGSQELPR | SEQ ID NO: 144 |
| | | | | QEPSQGTTTFAVTSILR | SEQ ID NO: 95 |
| | | | | DASGVTFTWTPSSGK | SEQ ID NO: 19 |
| BF-146 | BPI-156 | 5.32 | 63,088 | SAVQGPPER | SEQ ID NO: 107 |
| | | | | QEPSQGTTTFAVTSILR | SEQ ID NO: 95 |
| | | | | WLQGSQELPR | SEQ ID NO: 144 |
| | | | | YLTWASR | SEQ ID NO: 153 |
| BF-147 | BPI-158 | 5.03 | 64,235 | EVPLNTIIFMGR | SEQ ID NO: 43 |
| | | | | LPGIVAEGR | SEQ ID NO: 82 |
| BF-147 | BPI-159 | 5.03 | 64,235 | SAVQGPPER | SEQ ID NO: 107 |
| | | | | QEPSQGTTTFAVTSILR | SEQ ID NO: 95 |
| | | | | WLQGSQELPR | SEQ ID NO: 144 |
| | | | | YLTWASR | SEQ ID NO: 153 |
| | | | | DASGVTFTWTPSSGK | SEQ ID NO: 19 |
| BF-147 | BPI-160 | 5.03 | 64,235 | LALDNGGLAR | SEQ ID NO: 77 |
| | | | | SPEQQETVLDGNLIIR | SEQ ID NO: 116 |
| BF-148 | BPI-161 | 4.98 | 62,149 | VGEVLNSIFFELEADER | SEQ ID NO: 130 |
| | | | | ALQDQLVLVAAK | SEQ ID NO: 11 |
| | | | | SLDFTELDVAAEK | SEQ ID NO: 114 |
| | | | | QPFVQGLALYTPVVLPR | SEQ ID NO: 99 |
| | | | | DPTFIPAPIQAK | SEQ ID NO: 26 |
| BF-148 | BPI-162 | 4.98 | 62,149 | SPEQQETVLDGNLIIR | SEQ ID NO: 116 |
| BF-148 | BPI-163 | 4.98 | 62,149 | LPGIVAEGR | SEQ ID NO: 82 |
| BF-149 | BPI-164 | 4.53 | 59,743 | HTLNQIDEVK | SEQ ID NO: 63 |
| | | | | APHGPGLIYR | SEQ ID NO: 13 |
| BF-150 | BPI-165 | 5.52 | 53,454 | VTYTSQEDLVEK | SEQ ID NO: 139 |
| | | | | KVTYTSQEDLVEK | SEQ ID NO: 76 |
| BF-151 | BPI-167 | 4.55 | 50,129 | YLFLNGNK | SEQ ID NO: 152 |
| | | | | VAAGAFQGLR | SEQ ID NO: 127 |
| | | | | ALGHLDLSGNR | SEQ ID NO: 9 |
| | | | | DLLLPQPDLR | SEQ ID NO: 25 |
| | | | | ENQLEVLEVSWLHGLK | SEQ ID NO: 35 |
| BF-152 | BPI-170 | 4.10 | 44,095 | WFYIASAFR | SEQ ID NO: 143 |
| | | | | NWGLSVYADKPETTK | SEQ ID NO: 91 |
| | | | | SDVVYTDWK | SEQ ID NO: 108 |
| | | | | EQLGEFYEALDCLR | SEQ ID NO: 36 |
| BF-156 | BPI-172 | 6.88 | 39,900 | INHGILYDEEK | SEQ ID NO: 66 |
| | | | | EIMENYNIALR | SEQ ID NO: 32 |
| BF-157 | BPI-173 | 7.66 | 34,359 | TATSEYQTFFNPR | SEQ ID NO: 118 |
| | | | | ETAASLLQAGYK | SEQ ID NO: 40 |
| BF-157 | BPI-174 | 7.66 | 34,359 | SYSPYDMLESIR | SEQ ID NO: 117 |
| | | | | AYTNFDAER | SEQ ID NO: 18 |
| | | | | TNQELQEINR | SEQ ID NO: 124 |
| | | | | AEDGSVIDYELIDQDAR | SEQ ID NO: 4 |
| BF-158 | BPI-175 | 6.28 | 32,635 | QDGSVDFFR | SEQ ID NO: 92 |
| | | | | YGIDWASGR | SEQ ID NO: 151 |
| | | | | GEPGDPVNLLR | SEQ ID NO: 55 |
| BF-159 | BPI-176 | 5.65 | 31,545 | GSPAINVAVHVFR | SEQ ID NO: 59 |
| | | | | AADDTWEPFASGK | SEQ ID NO: 1 |
| BF-159 | BPI-177 | 5.65 | 31,545 | VTIGLLFWDGR | SEQ ID NO: 138 |
| | | | | ETLFSVMPGLK | SEQ ID NO: 41 |
| BF-160 | BPI-178 | 6.74 | 27,296 | YAASSYLSLTPEQWK | SEQ ID NO: 146 |
| BF-161 | BPI-179 | 5.15 | 25,593 | YEVQGEVFTK | SEQ ID NO: 149 |
| | | | | YEVQGEVFTKPQLWP | SEQ ID NO: 150 |
| | | | | RQDNEILIFWSK | SEQ ID NO: 103 |
| | | | | QDNEILIFWSK | SEQ ID NO: 93 |
| | | | | GYSIFSYATK | SEQ ID NO: 60 |
| BF-162 | BPI-180 | 6.73 | 24,401 | TVAAPSVFIFPPSDEQLK | SEQ ID NO: 126 |
| BF-163 | BPI-181 | 7.82 | 23,857 | LLIYDTSNR | SEQ ID NO: 80 |
| BF-163 | BPI-182 | 7.82 | 23,857 | TVAAPSVFIFPPSDEQLK | SEQ ID NO: 126 |
| | | | | FSGSGSGTDFTLK | SEQ ID NO: 50 |
| BF-164 | BPI-190 | 5.52 | 22,177 | FLVGPDGIPIMR | SEQ ID NO: 47 |
| | | | | QEPGENSEILPTLK | SEQ ID NO: 94 |
| BF-165 | BPI-184 | 6.00 | 15,113 | QPVPGQQMTLK | SEQ ID NO: 100 |
| | | | | IWDVVEK | SEQ ID NO: 73 |
| | | | | LVAYYTLIGASGQR | SEQ ID NO: 85 |
| | | | | EVVADSVWVDVK | SEQ ID NO: 44 |
| BF-509 | BPI-514 | 5.47 | 57,934 | YTFELSR | SEQ ID NO: 154 |
| | | | | THLPEVFLSK | SEQ ID NO: 121 |
| BF-510 | BPI-516 | 4.47 | 51,499 | YLFLNGNK | SEQ ID NO: 152 |
| | | | | VAAGAFQGLR | SEQ ID NO: 127 |
| | | | | ALGHLDLSGNR | SEQ ID NO: 9 |
| | | | | DLLLPQPDLR | SEQ ID NO: 25 |
| BF-511 | BPI-517 | 4.62 | 44,998 | YLFLNGNK | SEQ ID NO: 152 |
| | | | | DLLLPQPDLR | SEQ ID NO: 25 |
| BF-512 | BPI-521 | 5.57 | 92,686 | RVWELSK | SEQ ID NO: 106 |
| | | | | LPGIVAEGR | SEQ ID NO: 82 |
| | | | | DDLYVSDAFHK | SEQ ID NO: 20 |
| BF-513 | BPI-523 | 5.96 | 79,323 | EVPLNTIIFMGR | SEQ ID NO: 43 |
| | | | | LPGIVAEGR | SEQ ID NO: 82 |
| | | | | DDLYVSDAFHK | SEQ ID NO: 20 |
| BF-513 | BPI-545 | 5.96 | 79,323 | QIQVSWLR | SEQ ID NO: 96 |
| | | | | DGFFGNPR | SEQ ID NO: 22 |
| | | | | VSVFVPPR | SEQ ID NO: 137 |
| | | | | YVTSAPMPEPQAPGR | SEQ ID NO: 156 |
| BF-514 | BPI-527 | 5.11 | 65,901 | RVWELSK | SEQ ID NO: 106 |
| | | | | LPGIVAEGR | SEQ ID NO: 82 |
| | | | | DDLYVSDAFHK | SEQ ID NO: 20 |
| BF-515 | BPI-529 | 5.07 | 63,379 | EVPLNTIIFMGR | SEQ ID NO: 43 |
| | | | | LPGIVAEGR | SEQ ID NO: 82 |
| | | | | DDLYVSDAFHK | SEQ ID NO: 20 |
| BF-516 | BPI-531 | 5.17 | 61,951 | RVWELSK | SEQ ID NO: 106 |
| | | | | EVPLNTIIFMGR | SEQ ID NO: 43 |
| | | | | LPGIVAEGR | SEQ ID NO: 82 |
| BF-516 | BPI-546 | 5.17 | 61,951 | FAHTVVTSR | SEQ ID NO: 46 |
| | | | | SPEQQETVLDGNLIIR | SEQ ID NO: 116 |
| BF-517 | BPI-532 | 4.85 | 61,074 | LPGIVAEGR | SEQ ID NO: 82 |
| | | | | DDLYVSDAFHK | SEQ ID NO: 20 |
| BF-518 | BPI-533 | 5.34 | 60,714 | EVPLNTIIFMGR | SEQ ID NO: 43 |
| | | | | VAEGTQVLELPFK | SEQ ID NO: 128 |
| | | | | DDLYVSDAFHK | SEQ ID NO: 20 |
| BF-519 | BPI-534 | 5.30 | 57,026 | YTFELSR | SEQ ID NO: 154 |
| | | | | THLPEVFLSK | SEQ ID NO: 121 |
| | | | | RTHLPEVFLSK | SEQ ID NO: 104 |
| BF-519 | BPI-535 | 5.30 | 57,026 | LPGIVAEGR | SEQ ID NO: 82 |
| | | | | DDLYVSDAFHK | SEQ ID NO: 20 |
| BF-520 | BPI-536 | 4.96 | 46,200 | VAEGTQVLELPFK | SEQ ID NO: 128 |
| | | | | DDLYVSDAFHK | SEQ ID NO: 20 |

Four subsets of BPIs were identified as follows:

List 5: BPIs decreased in serum from subjects with primary breast cancer: BF-103, BF-106, BF-111, BF-112, BF-114, BF-115, BF-117, BF-118, BF-119, BF-122, BF-126, BF-127, BF-127, BF-128, BF-130, BF-131, BF-132, BF-132, BF-134, BF-135.

List 6: BPIs increased in serum from subjects with primary breast cancer: BF-101, BF-102, BF-104, BF-105, BF-107, BF-108, BF-108, BF-108, BF-109, BF-110, BF-113, BF-116, BF-120, BF-121, BF-123, BF-124, BF-125, BF-129, BF-133, BF-133, BF-136.

List 7: BPIs decreased in serum form subjects with metastatic breast cancer: BPI-129, BPI-130, BPI-131, BPI-138, BPI-139, BPI-143, BPI-144, BPI-146, BPI-152, BPI-153, BPI-155, BPI-156, BPI-158, BPI-159, BPI-160, BPI-161, BPI-162, BPI-163, BPI-164, BPI-165, BPI-173, BPI-174, BPI-175, BPI-176, BPI-177, BPI-178, BPI-180, BPI-190, BPI-184.

List 8 BPIs increased in serum form subjects with metastatic breast cancer: BPI-192, BPI-133, BPI-135, BPI-145, BPI-147, BPI-148, BPI-149, BPI-150, BPI-154, BPI-167, BPI-170, BPI-172, BPI-179, BPI-181, BPI-182.

Two clusters of BPIs were identified:

Cluster III: BPI-104, BPI-103, BPI-130, BPI-149, BPI-150, BPI-158, BPI-521, BPI-523, BPI-527, BPI-529, BPI-531, BPI-532, BPI-533, BPI-534, BPI-535, BPI-536

Cluster IV: BPI-130, BPI-167, BPI-173, BPI-174, BPI-514, BPI-516, BPI-517

As will be evident to one of skill in the art, based upon the present description, a given BPI can be described according to the data provided for that BPI in Table III. The BPI is a polypeptide comprising a peptide sequence described for that BPI (preferably comprising a plurality of, more preferably all of, the peptide sequences described for that BPI) and has a pI of about the value stated for that BPI (preferably within 10%, more preferably within 5% still more preferably within 1% of the stated value) and has a MW of about the value stated for that BPI (preferably within 10%, more preferably within 5%, still more preferably within 1% of the stated value).

In one embodiment, a first biological sample from a subject is analysed for quantitative detection of one or more of the BPIs as defined in List 5, or any combination of them, wherein a decreased abundance of the BPI or BPIs (or any combination of them) in the first sample from the subject relative to the second sample from a subject or subjects free from breast cancer (e.g., a control sample or a previously determined reference range) indicates the presence of primary breast cancer.

In another embodiment of the invention, a first biological sample from a subject is analysed for quantitative detection of one or more of the BPIs as defined in List 6, or any combination of them, wherein an increased abundance of the BPI or BPIs (or any combination of them) in the first sample from the subject relative to the second sample from a subject or subjects free from breast cancer (e.g., a control sample or a previously determined reference range) indicates the presence of primary breast cancer.

In a further embodiment, a first biological sample from a subject is analysed for quantitative detection of (a) one or more BPIs, or any combination of them, whose decreased abundance indicates the presence of primary breast cancer, i.e., the BPIs as defined in List 5; and (b) one or more BPIs, or any combination of them, whose increased abundance indicates the presence of primary breast cancer, i.e., the BPIs as defined in List 6.

In yet a further embodiment, a first biological sample from a subject is analysed for quantitative detection of one or more BPIs (as defined in Lists 5 and 6) and one or more previously known biomarkers of breast cancer (e.g., the extracellular domain of the HER-2/neu oncogene product (Payne RC et al, Clin Chem 2000;46(2):175-82)). In accordance with this embodiment, the abundance of each BPI and known biomarker relative to a control or reference range indicates whether a subject has primary breast cancer.

In yet another embodiment, a first biological sample from a subject is analysed for quantitative detection of one or more of the BPIs as defined in List 7, or any combination of them, wherein a decreased abundance of the BPI or BPIs (or any combination of them) in the first sample from the subject relative to the second sample from a subject or subjects free from breast cancer (e.g., a control sample or a previously determined reference range) indicates the presence of metastatic breast cancer.

In yet another embodiment of the invention, a first biological sample from a subject is analysed for quantitative detection of one or more of the BPIs as defined in List 8, or any combination of them, wherein an increased abundance of the BPI or BPIs (or any combination of them) in the first sample from the subject relative to the second sample from a subject or subjects free from breast cancer (e.g., a control sample or a previously determined reference range) indicates the presence of metastatic breast cancer.

In a further embodiment, a first biological sample from a subject is analysed for quantitative detection of (a) one or more BPIs, or any combination of them, whose decreased abundance indicates the presence of metastatic breast cancer, i.e., the BPIs as defined in List 7; and (b) one or more BPIs, or any combination of them, whose increased abundance indicates the presence of metastatic breast cancer, i.e., the BPIs as defined in List 8.

In yet a further embodiment, a first biological sample from a subject is analysed for quantitative detection of one or more BPIs (as defined in Lists 7 and 8) and one or more previously known biomarkers of breast cancer (e.g., the extracellular domain of the HER-2/neu oncogene product Payne RC et al, Clin Chem 2000;46(2): 175-82; or the biomarkers described in WO 01/13117). In accordance with this embodiment, the abundance of each BPI and known biomarker relative to a control or reference range indicates whether a subject has metastatic breast cancer.

In a preferred embodiment, a first biological sample from a subject is analysed for quantitative detection of one or more BPIs comprising a cluster as defined in Cluster III or Cluster IV above and in the examples infra, wherein an altered abundance of one or more of the BPIs comprising the said cluster in the first sample from the subject relative to the second sample from a subject or subjects free from breast cancer (e.g., a control sample or a previously determined reference range) indicates the presence of breast cancer and more specifically the sub-type of breast cancer.

In a further embodiment, a first biological sample from a subject is analysed for quantitative detection of one or more BPIs comprising a cluster as defined in Cluster III or Cluster IV above and in the examples infra, and one or more known biomarkers for breast cancer (e.g. the extracellular domain of the HER-2/neu oncogene product Payne RC et al, Clin Chem 2000;46(2): 175-82) In accordance with this embodiment, the abundance of each BPI and known biomarker relative to a control or reference range indicates whether a subject has breast cancer and more particularly the sub-type of breast cancer.

Preferably, the abundance of a BPI is normalised to an Expression Reference Protein Isoform (ERPI). ERPIs can be identified by partial amino acid sequencing of ERFs, which are described above, using the methods and apparatus of the Preferred Technology. The partial amino acid sequences of ERPIs are presented in Table IV.

TABLE IV
Expression Reference Protein Isoforms

| | | | | Amino Acid | |
|---|---|---|---|---|---|
| | | | | Sequences of | |
| Feature | Isoform | | | Tryptic Digest | |
| (BF) | (BPI) | pI | MW (Da) | Peptides | SEQ ID NO |
| ERF-2 | ERPI-1 | 5.03 | 30780 | HHGPTITAK | SEQ ID NO: 61 |
| | | | | ETLLQDFR | SEQ ID NO: 42 |

The BPIs described herein include isoforms of known proteins where the isoforms were not previously known to be associated with breast cancer. For each BPI, the present invention additionally provides: (a) antibodies that bind to said BPI, to said fragments, or both to said BPI and to said fragments. Preferably the BPI is in an isolated form. A contaminating protein is a protein or protein isoform having a significantly different pI or MW from those of the isolated BPI, as determined by 2D electrophoresis. As used herein, a “significantly different” pI or MW is one that permits the contaminating protein to be resolved from the BPI on 2D electrophoresis, performed according to the Reference Protocol.

In one embodiment, an isolated polypeptide is provided, said polypeptide comprising a peptide with the amino acid sequence identified in Table III for a BPI, said polypeptide having a pI and MW within 10% (preferably within 5%, more preferably within 1%) of the values identified in Table III for that BPI.

The BPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the BPIs are separated on a 2-D gel by virtue of their MWs and pIs and visualized by staining the gel. In one embodiment, the BPIs are stained with a fluorescent dye and imaged with a fluorescence scanner. Sypro Red (Molecular Probes, Inc., Eugene, Oreg.) is a suitable dye for this purpose. A preferred fluorescent dye is Pyridinium, 4-[2-[4-(dipentylamino)-2-trifluoromethylphenyl]ethenyl]-1-(sulfobutyl)-, inner salt. See U.S. Pat. No. 6,335,446, which is incorporated herein by reference in its entirety.

Alternatively, BPIs can be detected in an immunoassay. In one embodiment, an immunoassay is performed by contacting a first sample from a subject to be tested with a capture reagent (e.g. an antibody) under conditions such that immunospecific binding can occur if the BPI is present, and detecting or measuring the amount of any immunospecific binding by the capture reagent. Anti-BPI antibodies can be produced by the methods and techniques taught herein; examples of such antibodies known in the art are set forth in Table V. These antibodies shown in Table V are already known to bind to the protein of which the BPI is itself a family member. Preferably, the anti-BPI antibody preferentially binds to the BPI rather than to other isoforms of the same protein. In a preferred embodiment, the anti-BPI antibody binds to the BPI with at least 2-fold greater affinity, more preferably at least 5-fold greater affinity, still more preferably at least 10-fold greater affinity, than to said other isoforms of the same protein. When the antibodies shown in Table V do not display the required preferential selectivity for the target BPI, one skilled in the art can generate additional antibodies by using the BPI itself for the generation of such antibodies.

BPIs can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-BPI antibodies as described herein, e.g., the antibodies identified in Table V, or others raised against the BPIs of interest as those skilled in the art will appreciate based on the present description. The immunoblots can be used to identify those anti-BPI antibodies displaying the selectivity required to immuno-specifically differentiate a BPI from other isoforms encoded by the same gene.

TABLE V
Known Antibodies That Recognise BPIs or BPI-Related Polypeptides

| Feature (BF) | Isoform (BPI) | Antibody | Manufacturer | Cat. No. |
|---|---|---|---|---|
| BF-104 | BPI-186 | C6 Complement, Goat | ACCURATE CHEMICAL & | BMD-G33 |
| | | anti-Human | SCIENTIFIC | |
| | | | CORPORATION | |
| BF-107 | BPI-187 | C3 Complement, | ACCURATE CHEMICAL & | IMS-01-001- |
| | | Chicken anti-Human | SCIENTIFIC | 02 |
| | | | CORPORATION | |
| BF-118 | BPI-191 | Apolipoprotein A1 | ACCURATE CHEMICAL & | ACL- |
| | | (HDL), Sheep anti- | SCIENTIFIC | 20075AP |
| | | Human | CORPORATION | |
| BF-128 | BPI-189 | Sheep anti-Human | BIODESIGN | K90097C |
| | | Glutathione | INTERNATIONAL | |
| | | Peroxidase/Catalase | | |
| BF-131 | BPI-129 | Hemopexin, Beta-1, | ACCURATE CHEMICAL & | YN-RHHPX |
| | | Rabbit anti-Human, | SCIENTIFIC | |
| | | precipitating | CORPORATION | |
| BF-132 | BPI-130 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-133 | BPI-135 | Monoclonal mouse anti- | RDI RESEARCH | RDI-TRK1A2- |
| | | human IgA1 | DIAGNOSTICS, INC | 2B5 |
| BF-134 | BPI-138 | Transthyretin, | ACCURATE CHEMICAL & | MED-CLA |
| | | Prealbumin, 55 kD, | SCIENTIFIC | 193 |
| | | Rabbit anti-Human | CORPORATION | |
| BF-135 | BPI-139 | Apolipoprotein A1 | ACCURATE CHEMICAL & | ACL- |
| | | (HDL), Sheep anti- | SCIENTIFIC | 20075AP |
| | | Human | CORPORATION | |
| BF-137 | BPI-143 | Chicken polyclonal to | Abcam Ltd. | ab453-1000 |
| | | human mu-chain | | |
| BF-137 | BPI-144 | rabbit anti-human alpha- | Cambio Ltd. | CA-0427 |
| | | 2-macroglobulin | | |
| BF-140 | BPI-148 | Complement Factor B, | ACCURATE CHEMICAL & | AXL-466/2 |
| | | C3 proactivator, Rabbit | SCIENTIFIC | |
| | | anti-Human | CORPORATION | |
| BF-141 | BPI-149 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-141 | BPI-150 | Prothrombin, Rabbit | ACCURATE CHEMICAL & | AXL-448/2 |
| | | anti-Human | SCIENTIFIC | |
| | | | CORPORATION | |
| BF-144 | BPI-154 | Alpha-1- | ACCURATE CHEMICAL & | AXL-145/2 |
| | | Antichymotrypsin, | SCIENTIFIC | |
| | | Rabbit anti-Human | CORPORATION | |
| BF-145 | BPI-155 | Monoclonal mouse anti- | RDI RESEARCH | RDI-TRK1A2- |
| | | human IgA1 | DIAGNOSTICS, INC | 2B5 |
| BF-146 | BPI-156 | Monoclonal mouse anti- | RDI RESEARCH | RDI-TRK1A2- |
| | | human IgA1 | DIAGNOSTICS, INC | 2B5 |
| BF-147 | BPI-158 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-147 | BPI-159 | Monoclonal mouse anti- | RDI RESEARCH | RDI-TRK1A2- |
| | | human IgA1 | DIAGNOSTICS, INC | 2B5 |
| BF-148 | BPI-163 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-148 | BPI-161 | AT1 (306) | SANTA CRUZ | sc-579 |
| | | | BIOTECHNOLOGY, INC - | |
| | | | RESEARCH ANTIBODIES | |
| | | | 98/99 | |
| BF-152 | BPI-170 | Rabbit Polyclonal Anti- | Biogenesis Ltd. | 4729-9957 |
| | | Human alpha-1-acid- | | |
| | | Glycoprotein | | |
| BF-156 | BPI-172 | Factor H (Complement), | ACCURATE CHEMICAL & | IMS-01-066- |
| | | Chicken anti-Human | SCIENTIFIC | 02 |
| | | | CORPORATION | |
| BF-157 | BPI-173 | Prothrombin, Rabbit | ACCURATE CHEMICAL & | AXL-448/2 |
| | | anti-Human | SCIENTIFIC | |
| | | | CORPORATION | |
| BF-157 | BPI-174 | Rabbit anti-Annexin II | BIODESIGN | K80100R |
| | | monomer | INTERNATIONAL | |
| BF-159 | BPI-176 | Transthyretin, | ACCURATE CHEMICAL & | MED-CLA |
| | | Prealbumin, 55 kD, | SCIENTIFIC | 193 |
| | | Rabbit anti-Human | CORPORATION | |
| BF-164 | BPI-190 | Sheep anti-Human | BIODESIGN | K90097C |
| | | Glutathione | INTERNATIONAL | |
| | | Peroxidase/Catalase | | |
| BF-165 | BPI-184 | C3 Complement, | ACCURATE CHEMICAL & | IMS-01-001- |
| | | Chicken anti-Human | SCIENTIFIC | 02 |
| | | | CORPORATION | |
| BF-512 | BPI-521 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-513 | BPI-523 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-513 | BPI-545 | Chicken polyclonal to | Abcam Ltd. | ab453-1000 |
| | | human mu-chain | | |
| BF-514 | BPI-527 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-515 | BPI-529 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-516 | BPI-531 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-517 | BPI-532 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-518 | BPI-533 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-519 | BPI-535 | Antithrombin III, Clone: | ACCURATE CHEMICAL & | BYA-9009-1 |
| | | BL-ATIII/3, mAb anti- | SCIENTIFIC | |
| | | Human | CORPORATION | |
| BF-520 | BPI-536 | Antithrombin III, Clone: | ACCURATE CHEMICAL & | BYA-9009-1 |
| | | BL-ATIII/3, mAb anti- | SCIENTIFIC | |
| | | Human | CORPORATION | |

**Further information about these antibodies can be obtained from their commercial sources at: ACCURATE CHEMICAL & SCIENTIFIC CORPORATION - http://www.accuratechemical.com/; BIODESIGN INTERNATIONAL - http://www.biodesign.com/; DAKO CORPORATION - http://www.dakousa.com; RDI RESEARCH DIAGNOSTICS, INC - http://www.researchd.com/; SANTA CRUZ BIOTECHNOLOGY, INC - http://www.scbt.com/.

In one embodiment, binding of antibody in tissue sections can be used to detect aberrant localisation or expression levels of one or more BPIs. In a further embodiment the binding of an antibody in tissue sections can be used to detect the aberrant localisation or expression levels of BPIs that comprise a cluster. In a specific embodiment, antibody to a BPI can be used to assay a first biological sample (e.g., serum) from a subject for the level of the BPI where an aberrant level of BPI is indicative of primary or metastatic breast cancer. In a further embodiment antibodies to a cluster of BPIs can be used to assay a first biological sample (e.g. serum sample) from a subject for the level of BPIs comprising the cluster wherein an aberrant level of BPIs comprising the cluster is indicative of breast cancer and more specifically the sub-type of breast cancer.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a BPI can be detected in a fluid sample (e.g., CSF, blood, urine, or tissue homogenate) by means of a two-step sandwich assay. In the first step, a capture reagent (e.g., an anti-BPI antibody) is used to capture the BPI. Examples of such antibodies known in the art are set forth in Table V. The capture reagent can optionally be immobilised on a solid phase. In the second step, a directly or indirectly labelled detection reagent is used to detect the captured BPI. In one embodiment, the detection reagent is a lectin. Any lectin can be used for this purpose that preferentially binds to the BPI rather than to other isoforms that have the same core protein as the BPI or to other polypeptides that share the antigenic determinant recognised by the antibody. In a preferred embodiment, the chosen lectin binds to the BPI with at least 2-fold greater affinity, more preferably at least 5-fold greater affinity, still more preferably at least 10-fold greater affinity, than to said other isoforms that have the same core protein as the BPI or to said other polypeptides that share the antigenic determinant recognised by the antibody. Based on the present description, a lectin that is suitable for detecting a given BPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the BPI in a 2D gel, in a replica of a 2D gel following transfer to a suitable solid substrate such as a nitrocellulose membrane, or in a two-step assay following capture by capture reagent. In an alternative embodiment, the detection reagent is an antibody, e.g., an antibody that immunospecifically detects post-translational modifications, such as an antibody that immunospecifically binds to phosphorylated amino acids. Examples of such antibodies include those that bind to phosphotyrosine (BD Transduction Laboratories, 2002, catalogue nos.: P11120; P39020), those that bind to phosphoserine (Zymed Laboratories Inc. 2002, South San Francisco, Calif., catalogue no. 61-8100) and those that bind to phosphothreonine (Zymed Laboratories Inc., 2002, South San Francisco, Calif., catalogue nos. 71-8200, 13-9200).

If desired, a gene encoding a BPI, genes encoding BPIs which comprise a cluster, related genes, or related nucleic acid sequences or subsequences, including complementary sequences, can also be used in hybridisation assays. A nucleotide encoding a BPI, nucleotides encoding BPIs comprising a cluster, or subsequences thereof comprising at least 8 nucleotides, preferably at least 12 nucleotides, and most preferably at least 15 nucleotides can be used as a hybridisation probe. Hybridisation assays can be used for detection, prognosis, diagnosis, or monitoring of conditions, disorders, or disease states, associated with aberrant expression of genes encoding BPIs, or for differential diagnosis of subjects with signs or symptoms suggestive of breast cancer. In particular, such a hybridisation assay can be carried out by a method comprising contacting a subject's sample containing nucleic acid with a nucleic acid probe capable of hybridising to a DNA or RNA that encodes a BPI, under conditions such that hybridisation can occur, and detecting or measuring any resulting hybridisation. Nucleotides can be used for therapy of subjects having breast cancer, as described below.

The invention also provides diagnostic kits, comprising an anti-BPI antibody. In addition, such a kit may optionally comprise one or more of the following: (1) instructions for using the anti-BPI antibody for diagnosis, prognosis, therapeutic monitoring or any combination of these applications; (2) a labelled binding partner to the antibody; (3) a solid phase (such as a reagent strip) upon which the anti-BPI antibody is immobilised; and (4) a label or insert indicating regulatory approval for diagnostic, prognostic or therapeutic use or any combination thereof. If no labelled binding partner to the antibody is provided, the anti-BPI antibody itself can be labelled with a detectable marker, e.g., a chemiluminescent, enzymatic, fluorescent, or radioactive moiety. In another embodiment the diagnostic kit comprises a plurality of anti-BPI antibodies that bind to a plurality of BPIs which comprise a cluster.

The invention also provides a kit comprising a nucleic acid probe capable of hybridising to RNA encoding a BPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a BPI, such as by polymerase chain reaction (see, e.g., Innis et al., 1990, PCR Protocols, Academic Press, Inc., San Diego, Calif.), ligase chain reaction (see EP 320,308) use of Q replicase, cyclic probe reaction, or other methods known in the art.

Kits are also provided which allow for the detection of a plurality of BPIs that comprise a cluster or a plurality of nucleic acids each encoding a BPI comprising a cluster. A kit can optionally further comprise a predetermined amount of an isolated BPI protein or a nucleic acid encoding a BPI, e.g., for use as a standard or control.

Statistical Techniques for Identifying BPI and BF Clusters

The uni-variate differential analysis tools, such as fold changes, Wilcoxon rank sum test and t-test, are useful in identifying individual BFs or BPIs that are diagnostically associated with breast cancer or in identifying individual BPIs that regulate the disease process. In most cases, however, those skilled in the art appreciate that the disease process is associated with a combination of BFs or BPIs (and to be regulated by a combination of BPIs), rather than individual BFs and BPIs in isolation. The strategies for discovering such combinations of BFs and BPIs differ from those for discovering individual BFs and BPIs. In such cases, each individual BF and BPIs can be regarded as one variable and the disease can be regarded as a joint, multi-variate effect caused by interaction of these variables.

The following steps can be used to identify markers from data produced by the Preferred Technology.

The first step is to identify a collection of BFs or BPIs that individually show a significant aberrant expression in breast cancer. The association between the identified BFs or BPIs and breast cancer need not be as highly significant as is desirable when an individual BF or BPI is used as a diagnostic. Any of the tests discussed above (fold changes, Wilcoxon rank sum test, etc.) can be used at this stage. Once a suitable collection of BFs or BPIs has been identified, a sophisticated multi-variate analysis capable of identifying clusters can then be used to estimate the significant multivariate associations with breast cancer.

Linear Discriminant Analysis (LDA) is one such procedure, which can be used to detect significant association between a cluster of variables (i.e., BFs or BPIs) and breast cancer. In performing LDA, a set of weights is associated with each variable (i.e., BF or BPI) so that the linear combination of weights and the measured values of the variables can identify the disease state by discriminating between subjects having breast cancer and subjects free from breast cancer. Enhancements to the LDA allow stepwise inclusion (or removal) of variables to optimise the discriminant power of the model. The result of the LDA is therefore a cluster of BFs or BPIs, which can be used without limitation for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of BFs or BPIs can be identified by qualitative measures by comparing the percentage feature presence of a BF or BPI of a first sample or sample set (e.g., samples from diseased subjects) with the percentage feature presence of a BF or BPI in a second sample or sample set (e.g., samples from control subjects). The “percentage feature presence” of a BF or BPI is the percentage of samples in a sample set in which the BF or BPI is detectable by the detection method of choice. For example, if a BF is detectable in 95 percent of samples from diseased subjects, the percentage feature presence of that BF in that sample set is 95 percent. If only 5 percent of samples from non-diseased subjects have detectable levels of the same BF, detection of that BF in the sample of a subject would suggest that it is likely that the subject suffers from breast cancer.

In another embodiment of the invention, one skilled in the art may analyse a series of BFs or BPIs that show an aberrant expression in breast cancer and use them to perform multiple LDA analyses to identify a cluster of BFs or BPIs that can be used to discriminate between primary breast cancer, metastatic breast cancer and control subjects.

In a preferred embodiment, a plurality of the BFs listed in Table I are used to identify a cluster of BFs that can be used to discriminate between patients with primary breast cancer, metastatic breast cancer and control subjects. Examples of such clusters include, but are not limited to, the following combination of BFs: BF-108, BF-132, BF-141, BF-147, BF-512, BF-513, BF-514, BF-515, BF-516, BF-517, BF-518, BF-519, BF-520; or the following combination of BFs: BF-132, BF-151, BF-157, BF-509, BF-510, BF-511.

In another embodiment, the BPIs listed in Table III are used to identify a cluster of BPIs that can be used to discriminate between patients that have primary breast cancer or metastatic breast cancer and control subjects. Examples of clusters of BPIs include, but are not limited to, the following cluster of BPIs: BPI-130, BPI-167, BPI-173, BPI-174, BPI-514, BPI-516, BPI-517; or the following cluster of BPIs: BPI-104, BPI-103, BPI-130, BPI-149, BPI-150, BPI-158, BPI-521, BPI-523, BPI-527, BPI-529, BPI-531, BPI-532, BPI-533, BPI-534, BPI-535, BPI-536.

Use in Clinical Studies

The diagnostic methods and compositions of the present invention can assist in monitoring a clinical study, e.g. to evaluate drugs for therapy of breast cancer. In one embodiment, candidate molecules are tested for their ability to restore BF or BPI levels in a subject having breast cancer to levels found in subjects free from breast cancer or, in a treated subject to preserve BF or BPI levels at or near non-breast cancer values. The levels of one or more BFs or BPIs, or a plurality of BFs or BPIs which comprise a cluster (as defined in the examples) can be assayed.

In another embodiment, the methods and compositions of the present invention are used to screen candidates for a clinical study to identify individuals having breast cancer in particular individuals with particular sub-types of breast cancer, such individuals can then be either excluded from or included in the study or can be placed in a separate cohort for treatment or analysis.

Purification of BPIs

In particular aspects, the invention provides isolated mammalian BPIs, preferably human BPIs, and fragments thereof which comprise an antigenic determinant (i.e., can be recognised by an antibody) or which are otherwise functionally active, as well as nucleic acid sequences encoding the foregoing. “Functionally active” as used herein refers to material displaying one or more functional activities associated with a full-length (wild-type) BPI, e.g., binding to a BPI substrate or BPI binding partner, antigenicity (binding to an anti-BPI antibody), immunogenicity, enzymatic activity and the like.

In specific embodiments, the invention provides fragments of a BPI comprising at least 5 amino acids, at least 10 amino acids, at least 50 amino acids, or at least 75 amino acids. Fragments lacking some or all of the regions of a BPI are also provided, as are polypeptides (e.g., fusion proteins) comprising such fragments. Nucleic acids encoding the foregoing are provided.

Once a recombinant nucleic acid which encodes the BPI, BPI fragment, or a precursor of the BPI is identified, the gene product can be analysed. This is achieved by assays based on the physical or functional properties of the product, including radioactive labelling of the product followed by analysis by gel electrophoresis, immunoassay, etc.

The BPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the BPI is identified, the entire amino acid sequence of the BPI can be deduced from the nucleotide sequence of the gene coding region contained in the recombinant nucleic acid. As a result, the protein can be synthesised by standard chemical methods known in the art (e.g., see Hunkapiller et al., 1984, Nature 310:105-111).

In another alternative embodiment, native BPIs can be purified from natural sources, by standard methods such as those described above (e.g., immunoaffinity purification).

In a preferred embodiment, BPIs are isolated by the Preferred Technology described supra. For preparative-scale runs, a narrow-range “zoom gel” having a pH range of 2 pH units or les's is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated BPIs that can be recovered from the gel. When used in this way for preparative-scale runs, the Preferred Technology typically provides up to 100 ng, and can provide up to 1000 ng, of an isolated BPI in a single run. Those of skill in the art will appreciate that a zoom gel can be used in any separation strategy which employs gel isoelectric focusing.

The invention thus provides a BPI, BPI fragment, BPI-related polypeptide or the BPI-fusion protein; any of the foregoing can be produced by recombinant DNA techniques or by chemical synthetic methods.

Isolation of DNA Encoding a BPI

Specific embodiments for the cloning of a gene encoding a BPI are presented below by way of example and not of limitation.

The nucleotide sequences of the present invention, including DNA and RNA, and comprising a sequence encoding a BPI, BPI fragment, BPI-related polypeptide or the BPI-fusion protein, may be synthesised using methods known in the art, such as using conventional chemical approaches or polymerase chain reaction (PCR) amplification. The nucleotide sequences of the present invention also permit the identification and cloning of the gene encoding a BPI homolog or BPI ortholog including, for example, by screening cDNA libraries, genomic libraries or expression libraries.

For example, to clone a gene encoding a BPI by PCR techniques, anchored degenerate oligonucleotides (or a set of most likely oligonucleotides) can be designed for all BPI peptide fragments identified as part of the same protein. PCR reactions under a variety of conditions can be performed with relevant cDNA and genomic DNAs (e.g., from tissue or body fluid or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):3942; Dyer KD, Biotechniques, 1995, 19(4):550-2). Vectorette PCR may be performed with probes that are, for example, anchored degenerate oligonucleotides (or most likely oligonucleotides) coding for BPI peptide fragments, using as a template a genomic library or cDNA library pools.

Anchored degenerate oligonucleotides (and most likely oligonucleotides) can be designed for all BPI peptide fragments. These oligonucleotides may be labelled and hybridised to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding BPI, BPI fragment, BPI-related polypeptide or the BPI-fusion protein of the present invention are useful for their ability to hybridise selectively with complementary stretches of genes encoding other proteins. Depending on the application, a variety of hybridisation conditions may be employed to obtain nucleotide sequences at least 30%, 35%, 40%, 45%, 50%, 55%, 60%, 65%, 70%, 75%, 80%, 85%, 90%, 95% or 99% identical, or 100% identical, to the sequence of a nucleotide encoding a BPI.

For a high degree of selectivity, relatively stringent conditions are used to form the duplexes, such as low salt or high temperature conditions. Hybridisation conditions can also be rendered more stringent by the addition of increasing amounts of formamide, to destabilise the hybrid duplex. Thus, particular hybridisation conditions can be readily manipulated, and will generally be chosen depending on the desired results. In general, convenient hybridisation temperatures in the presence of 50% formamide are: 42° C. for a probe which is 95 to 100% identical to the fragment of a gene encoding a BPI, 37° C. for 90 to 95% identity and 32° C. for 70 to 90% identity.

In the preparation of genomic libraries, DNA fragments are generated, some of which will encode parts or the whole of a BPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D.M. (ed.), 1985, DNA Cloning: A Practical Approach, MRL Press, Ltd., Oxford, U.K. Vol. 1, II; Ausubel F. M. et al., eds., 1989, Current Protocols in Molecular Biology, Vol. 1, Green Publishing Associates, Inc., and John Wiley & sons, Inc., New York). The genomic library may be screened by nucleic acid hybridisation to labelled probe (Benton and Davis, 1977, Science 196:180; Grunstein and Hogness, 1975, Proc. Natl. Acad. Sci. U.S.A. 72:3961).

Based on the present description, the genomic libraries may be screened with labelled degenerate oligonucleotide probes corresponding to the amino acid sequence of any peptide of the BPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides in length.

In Table III above, some BPIs disclosed herein were found to correspond to isoforms of previously identified proteins encoded by genes whose sequences are publicly known. (Sequence analysis and protein identification of BPIs was carried out using the methods described in Section 6.1.14). To screen such a gene, any probe may be used that is complementary to the gene or its complement; preferably the probe is 10 nucleotides or longer, more preferably 15 nucleotides or longer. The SWISS-PROT and trEMBL databases (held by the Swiss Institute of Bioinformatics (SIB) and the European Bioinformatics Institue (EBI) which are available at http://www.expasy.com/) and the GenBank database (held by the National Institute of Health (NIH) which is available at http://www.ncbi.nlm.nih.gov/GenBank/) provide protein sequences for the BPIs listed in Table III under the following accession numbers and each sequence is incorporated herein by reference (see Table VI):

TABLE VI
Sequences encoding BPIs and BPI Related Proteins

| BF # | BPI # | Accession Number |
|---|---|---|
| BF-105 | BPI-101 | P00747 |
| BF-108 | BPI-102 | P01871 |
| BF-108 | BPI-103 | P00734 |
| BF-108 | BPI-104 | P01008 |
| BF-110 | BPI-111 | AAD33873 |
| BF-112 | BPI-113 | 6996428 |
| BF-114 | BPI-114 | Q13906 |
| BF-115 | BPI-115 | P15169 |
| BF-116 | BPI-117 | P13645 |
| BF-117 | BPI-118 | P06727 |
| BF-119 | BPI-119 | P15169 |
| BF-120 | BPI-120 | P01024 |
| BF-122 | BPI-121 | P10909 |
| BF-123 | BPI-123 | P05090 |
| BF-124 | BPI-124 | P01024 |
| BF-126 | BPI-125 | 3659942 |
| BF-127 | BPI-126 | 3659942 |
| BF-127 | BPI-127 | 224377 |
| BF-131 | BPI-129 | P02790 |
| BF-132 | BPI-130 | P01008 |
| BF-132 | BPI-131 | Q14624 |
| BF-133 | BPI-133 | P02749 |
| BF-133 | BPI-135 | P01876 |
| BF-134 | BPI-138 | P02766 |
| BF-135 | BPI-139 | P02647 |
| BF-137 | BPI-143 | P01871 |
| BF-137 | BPI-144 | P01023 |
| BF-138 | BPI-145 | P08603 |
| BF-139 | BPI-146 | P01031 |
| BF-140 | BPI-147 | Q92489 |
| BF-140 | BPI-148 | P00751 |
| BF-141 | BPI-149 | P01008 |
| BF-141 | BPI-150 | P00734 |
| BF-142 | BPI-152 | P43652 |
| BF-143 | BPI-153 | Q14624 |
| BF-144 | BPI-154 | P01011 |
| BF-145 | BPI-155 | P01876 |
| BF-146 | BPI-156 | P01876 |
| BF-147 | BPI-158 | P01008 |
| BF-147 | BPI-159 | P01876 |
| BF-147 | BPI-160 | Q14624 |
| BF-148 | BPI-161 | P01019 |
| BF-148 | BPI-162 | Q14624 |
| BF-148 | BPI-163 | P01008 |
| BF-149 | BPI-164 | O14502 |
| BF-150 | BPI-165 | P05156 |
| BF-151 | BPI-167 | P02750 |
| BF-152 | BPI-170 | P02763 |
| BF-156 | BPI-172 | Q03591 |
| BF-157 | BPI-173 | P00734 |
| BF-157 | BPI-174 | P07355 |
| BF-158 | BPI-175 | O75636 |
| BF-159 | BPI-176 | P02766 |
| BF-159 | BPI-177 | Q14624 |
| BF-160 | BPI-178 | CAA40959 |
| BF-161 | BPI-179 | P02741 |
| BF-162 | BPI-180 | AAB86466 |
| BF-163 | BPI-181 | AAD19495 |
| BF-163 | BPI-182 | AAB51622 |
| BF-165 | BPI-184 | P01024 |
| BF-104 | BPI-186 | P13671 |
| BF-107 | BPI-187 | P01024 |
| BF-109 | BPI-188 | 189687 |
| BF-128 | BPI-189 | P22352 |
| BF-164 | BPI-190 | P22352 |
| BF-118 | BPI-191 | P06727 |
| BF-129 | BPI-192 | P13671 |
| BF-509 | BPI-514 | P02774 |
| BF-510 | BPI-516 | P02750 |
| BF-511 | BPI-517 | P02750 |
| BF-512 | BPI-521 | P01008 |
| BF-513 | BPI-523 | P01008 |
| BF-514 | BPI-527 | P01008 |
| BF-515 | BPI-529 | P01008 |
| BF-516 | BPI-531 | P01008 |
| BF-517 | BPI-532 | P01008 |
| BF-518 | BPI-533 | P01008 |
| BF-519 | BPI-534 | P02774 |
| BF-519 | BPI-535 | P01008 |
| BF-520 | BPI-536 | P01008 |

For any BPI, degenerate probes, or probes taken from the sequences described above by accession number may be used for screening. In the case of degenerate probes, they can be constructed from the partial amino sequence information obtained from tandem mass spectra of tryptic digest peptides of the BPI. To screen such a gene, any probe may be used that is complementary to the gene or its complement; the probe is 10 nucleotides or longer, preferably 15 nucleotides or longer. When a library is screened, clones with insert DNA encoding the BPI or a fragment thereof will hybridise to one or more members of the corresponding set of degenerate oligonucleotide probes (or their complement). Hybridisation of such oligonucleotide probes to genomic libraries is carried out using methods known in the art. For example, hybridisation with one of the above-mentioned degenerate sets of oligonucleotide probes, or their complement (or with any member of such a set, or its complement) can be performed under highly stringent or moderately stringent conditions as defined supra, or can be carried out in 2×SSC, 1.0% SDS at 50° C. and washed using the washing conditions described supra for highly stringent or moderately stringent hybridisation.

In yet another aspect of the invention, clones containing nucleotide sequences encoding the BPI, BPI fragment, BPI-related polypeptide or the BPI-fusion protein any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed BPI, BPI fragment, BPI-related polypeptide or the BPI-fusion protein. In one embodiment, the various anti-BPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes BPI, BPI fragment, BPI-related polypeptide or the BPI-fusion protein can be detected using DYNA Beads according to Olsvick et al., 29th ICAAC, Houston, Tex. 1989, incorporated herein by reference. Anti-BPI antibodies are crosslinked to tosylated DYNA Beads M280, and these antibody-containing beads are then contacted with colonies or plaques expressing recombinant polypeptides. Colonies or plaques expressing a BPI, BPI fragment, BPI-related polypeptide or the BPI-fusion protein are identified as any of those that bind the beads.

Alternatively, the anti-BPI antibodies can be non-specifically immobilised to a suitable support, such as silica or Celite™ resin. This material is then used to adsorb to bacterial colonies expressing the BPI, BPI fragment, BPI-related polypeptide or the BPI-fusion protein as described herein.

In another aspect, PCR amplification may be used to isolate from genomic DNA a substantially pure DNA (i.e., a DNA substantially free of contaminating nucleic acids) encoding the entire BPI or a part thereof. Preferably such a DNA is at least 95% pure, more preferably at least 99% pure. Oligonucleotide sequences, degenerate or otherwise, that correspond to peptide sequences of BPIs disclosed herein can be used as primers.

PCR can be carried out, e.g., by use of a Perkin-Elmer Cetus thermal cycler and Taq polymerase (Gene Amp™ or AmpliTaq™ DNA polymerase). One can choose to synthesise several different degenerate primers, for use in the PCR reactions. It is also possible to vary the stringency of hybridisation conditions used in priming the PCR reactions, to allow for greater or lesser degrees of nucleotide sequence similarity between the degenerate primers and the corresponding sequences in the DNA. After successful amplification of a segment of the sequence encoding a BPI, that segment may be molecularly cloned and sequenced, and utilised as a probe to isolate a complete genomic clone. This, in turn, will permit the determination of the gene's complete nucleotide sequence, the analysis of its expression, and the production of its protein product for functional analysis, as described infra.

The gene encoding a BPI can also be identified by mRNA selection by nucleic acid hybridisation followed by in vitro translation. In this procedure, fragments are used to isolate complementary mRNAs by hybridisation. Such DNA fragments may represent available, purified DNA encoding a BPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilised antibodies that specifically recognise a BPI. A radiolabelled cDNA encoding a BPI can be synthesised using the selected mRNA (from the adsorbed polysomes) as a template. The radiolabelled mRNA or cDNA may then be used as a probe to identify the DNA fragments encoding a BPI from among other genomic DNA fragments.

Alternatives to isolating genomic DNA encoding a BPI include, but are not limited to, chemically synthesising the gene sequence itself from a known sequence or making cDNA to the mRNA which encodes the BPI. For example, RNA for cDNA cloning of the gene encoding a BPI can be isolated from cells which express the BPI. Those skilled in the art will understand from the present description that other methods may be used and are within the scope of the invention.

Any suitable eukaryotic cell can serve as the nucleic acid source for the molecular cloning of the gene encoding a BPI. The nucleic acid sequences encoding the BPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. 1, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript™ vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesised oligonucleotides encoding restriction endonuclease recognition sequences. In an alternative method, the cleaved vector and the gene encoding a BPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the BPI, cDNA, or synthesised DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native BPI, nucleotide sequences encoding amino acid sequences with functionally equivalent amino acids, nucleotide sequences encoding BPI, BPI fragment, BPI-related polypeptide or the BPI-fusion protein.

In a specific embodiment, an isolated nucleic acid molecule encoding a BPI-related polypeptide can be created by introducing one or more nucleotide substitutions, additions or deletions into the nucleotide sequence of a BPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding BPIs

The nucleotide sequence coding for a BPI, BPI fragment or BPI-related polypeptide or other derivative of any of the foregoing, can be inserted into an appropriate expression vector, i.e., a vector, which contains the necessary elements for the transcription and translation of the inserted protein-coding sequence. The necessary transcriptional and translational signals can also be supplied by the native gene encoding the BPI or its flanking regions, or the native gene encoding the BPI-related polypeptide or its flanking regions. A variety of host-vector systems may be utilised in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilised, any one of a number of suitable transcription and translation elements may be used. In specific embodiments, a nucleotide sequence encoding a human gene (or a nucleotide sequence encoding a functionally active portion of a human BPI) is expressed. In yet another embodiment, a fragment of a BPI comprising a domain of the BPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding a BPI or fragment thereof may be regulated by a second nucleic acid sequence so that the BPI or fragment is expressed in a host transformed with the recombinant DNA molecule. For example, expression of a BPI may be controlled by any promoter or enhancer element known in the art. Promoters which may be used to control the expression of the gene encoding a BPI or a BPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:3942), the tetracycline (Tet) promoter (Gossen et al., 1995, Proc. Nat. Acad. Sci. USA 89:5547-5551); prokaryotic expression vectors such as the b-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus ³⁵S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumour virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5): 619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a BPI-encoding nucleic acid, one or more origins of replication, and, optionally, one or more selectable markers (e.g., an antibiotic resistance gene).

In a specific embodiment, an expression construct is made by subcloning a BPI, BPI fragment or BPI-related polypeptide coding sequence into the EcoRI restriction site of each of the three pGEX vectors (Glutathione S-Transferase expression vectors; Smith and Johnson, 1988, Gene 7:3140). This allows for the expression of the BPI product or BPI-related polypeptide from the subclone in the correct reading frame.

In mammalian host cells, a number of viral-based expression systems may be utilised. In cases where an adenovirus is used as an expression vector, the BPI coding sequence or BPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a BPI, BPI fragment or BPI-related polypeptide can be identified by three general approaches: (a) nucleic acid hybridisation, (b) presence or absence of “marker” gene functions, and (c) expression of inserted sequences. In the first approach, the presence of a gene encoding a BPI inserted in an expression vector can be detected by nucleic acid hybridisation using probes comprising sequences that are homologous to an inserted gene encoding a BPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a BPI in the vector. For example, if the gene encoding the BPI is inserted within the marker gene sequence of the vector, recombinants containing the gene encoding the BPI insert can be identified by the absence of the marker gene function. In the third approach, recombinant expression vectors can be identified by assaying the gene product (i.e., BPI) expressed by the recombinant. Such assays can be based, for example, on the physical or functional properties of the BPI in in vitro assay systems, e.g., binding with anti-BPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered BPI or BPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, HEK293, 3T3, WI38. Furthermore, different vector/host expression systems may effect processing reactions to different extents.

For long-term, high-yield production of recombinant proteins, stable expression is preferred. For example, cell lines which stably express the BPI may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the BPI. Such engineered cell lines may be particularly useful in screening and evaluation of agents that affect the endogenous activity of the BPI.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150: 1); and hygro, which confers resistance to hygromycin (Santerre, et al., 1984, Gene 30:147) genes.

In other specific embodiments, the BPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., WO 96/22024 and WO 99/04813).

Nucleic acids encoding a BPI, a fragment of a BPI, a B PI-related polypeptide, or a fragment of a BPI-related polypeptide can fused to an epitope tag (e.g., the hemagglutinin (“HA”) tag or flag tag) to aid in detection and purification of the expressed polypeptide. For example, a system described by Janknecht et al., allows for the ready purification of non-denatured fusion proteins expressed in human cell lines (Janknecht et al., 1991, Proc. Natl. Acad. Sci. USA 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesiser.

Both cDNA and genomic sequences can be cloned and expressed.

Domain Structure of BPIs

Domains of some BPIs are known in the art and have been described in the scientific literature. Moreover, domains of a BPI can be identified using techniques known to those of skill in the art. For example, one or more domains of a BPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occurring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347,166). The SAPS program analyses polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, the skilled artisan can identify domains of a BPI having enzymatic or binding activity, and further can identify nucleotide sequences encoding such domains. These nucleotide sequences can then be used for recombinant expression of a BPI fragment that retains the enzymatic or binding activity of the BPI.

Based on the present description, the skilled artisan can identify domains of a BPI having enzymatic or binding activity, and further can identify nucleotide sequences encoding such domains. These nucleotide sequences can then be used for recombinant expression of BPI fragments that retain the enzymatic or binding activity of the BPI.

In one embodiment, a BPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A BPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay.

Production of Antibodies to BPIs

According to the invention a BPI, BPI fragment, BPI-related polypeptide or the BPI-fusion protein or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanised or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognise gene products of genes encoding BPIs are publicly available. For example, antibodies that can recognise these BPI, BPI fragment, BPI-related polypeptide or the BPI-fusion proteins include antibodies, which can be purchased from commercial sources. In another embodiment, methods known to those skilled in the art are used to produce antibodies that recognise a BPI, BPI fragment, BPI-related polypeptide or BPI-fusion protein or derivatives of any of the foregoing.

In one embodiment of the invention, antibodies to a specific domain of a BPI are produced. In a specific embodiment, hydrophilic fragments of a BPI are used as immunogens for antibody production.

In the production of antibodies, screening for the desired antibody can be accomplished by techniques known in the art, e.g. ELISA (enzyme-linked immunosorbent assay). For example, to select antibodies, which recognise a specific domain of a BPI, one may assay generated hybridomas for a product, which binds to a BPI fragment containing such domain. For selection of an antibody that specifically binds a first BPI homolog but which does not specifically bind to (or binds less avidly to) a second BPI homolog, one can select on the basis of positive binding to the first BPI homolog and a lack of binding to (or reduced binding to) the second BPI homolog. Similarly, for selection of an antibody that specifically binds a BPI but which does not specifically bind to (or binds less avidly to) a different isoform of the same protein (such as a different glycoform having the same core peptide as the BPI), one can select on the basis of positive binding to the BPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to a BPI than to a different isoform or isoforms (e.g., glycoforms) of the BPI.

Polyclonal antibodies, which may be used in the methods of the invention, are heterogeneous populations of antibody molecules derived from the sera of immunised animals. Unfractionated immune serum can also be used. Various procedures known in the art may be used for the production of polyclonal antibodies to a BPI, BPI fragment, BPI-related polypeptide or the BPI-fusion protein. In a particular embodiment, rabbit polyclonal antibodies to an epitope of a BPI, BPI fragment, BPI-related polypeptide or the BPI-fusion protein can be obtained. For example, for the production of polyclonal or monoclonal antibodies, various host animals can be immunised by injection with the native or a synthetic (e.g., recombinant) version of a BPI, BPI fragment, BPI-related polypeptide or the BPI-fusion protein, including but not limited to rabbits, mice, rats, etc. The Preferred Technology described herein provides isolated BPIs suitable for such immunisation. If the BPI is purified by gel electrophoresis, the BPI can be used for immunisation with or without prior extraction from the polyacrylamide gel. Various adjuvants may be used to enhance the immunological response, depending on the host species, including, but not limited to, complete or incomplete Freund's adjuvant, a mineral gel such as aluminium hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mabs) directed toward a BPI, BPI fragment, BPI-related polypeptide or the BPI-fusion protein, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, mAbs can be produced in germ-free animals utilising known technology (PCT/US90/02545).

The mabs include but are not limited to human mabs and chimeric mAbs (e.g., human-mouse chimeras). A chimeric antibody is a molecule in which different portions are derived from different animal species, such as those having a human immunoglobulin constant region and a variable region derived from a murine mAb. (See, e.g., U.S. Pat. No. 4,816,567 and U.S. Pat. No. 4,816,397). Humanised antibodies are antibody molecules from non-human species having one or more complementarily determining regions (CDRs) from the non-human species and a framework region from a human immunoglobulin molecule. (See, e.g. U.S. Pat. No. 5,585,089).

Chimeric and humanised mAbs can be produced by recombinant DNA techniques known in the art, for example using methods described in WO 87/02671; EP 184,187A; EP 171,496A; EP 173,494A; WO 86/01533; U.S. Pat. No. 4,816,567; EP 125,023A; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al., (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunised in the normal fashion with a selected antigen, e.g., all or a portion of a BPI of the invention. Monoclonal antibodies directed against the antigen can be obtained using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies, which recognise a selected epitope can be generated using a technique referred to as “guided selection”. In this approach a selected non-human monoclonal antibody, e.g., a mouse antibody, is used to guide the selection of a completely human antibody recognising the same epitope. (Jespers et al., (1994) Bio/technology 12:899-903).

The antibodies of the present invention can also be generated using various phage display methods known in the art. In phage display methods, functional antibody domains are displayed on the surface of phage particles which carry the polynucleotide sequences encoding them. In particular, such phage can be utilised to display antigen binding domains expressed from a repertoire or combinatorial antibody library (e.g., human or murine). Phage expressing an antigen binding domain that binds the antigen of interest can be selected or identified with antigen, e.g., using labelled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 1879-18 (1997); Burton et al., Advances in Immunology 57:191-280 (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in WO 92/22324; Mullinax et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the co-expression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J. 10:3655-3659.

According to a different and preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details for generating bispecific antibodies see, for example, Suresh et al., Methods in Enzymology, 1986, 121:210.

The invention provides functionally active fragments, derivatives or analogs of the anti-BPI immunoglobulin molecules. Functionally active means that the fragment, derivative or analog is able to elicit anti-anti-idiotype antibodies (i.e., tertiary antibodies) that recognise the same antigen that is recognised by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognises the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognise specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:42342; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatisation by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localisation and activity of the BPIs of the invention, e.g., for imaging these polypeptides, measuring levels thereof in appropriate physiological samples, in diagnostic methods, etc.

Expression of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridisable to the 3′ and 5′ ends of the sequence or by cloning using an oligonucleotide probe specific for the particular gene sequence.

If an antibody molecule that specifically recognises a particular antigen is not available (or a source for a cDNA library for cloning a nucleic acid encoding such an antibody), antibodies specific for a particular antigen may be generated by any method known in the art, for example, by immunising an animal, such as a rabbit, to generate polyclonal antibodies or, more preferably, by generating mAbs. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., WO 86/05807; WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydryl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCR based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanised antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods, which are well known to those skilled in the art, can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al., (1990, Molecular Cloning, A Laboratory Manual, 2d Ed., Cold Spring Harbor Laboratory, Cold Spring Harbor, N.Y.) and Ausubel et al., (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 198, Gene 45: 101; Cockett et al., 1990, Bio/Technology 8:2).

A variety of host-expression vector systems may be utilised to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK293, 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione-S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilised.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable marker (e.g., neomycin or hygromycin), and selecting for expression of the selectable marker. Such engineered cell lines may be particularly useful in screening and evaluation of agents that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilising an antibody specific for the fusion protein being expressed. For example, a system described by Janknecht et al., allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

Conjugated Antibodies

In a preferred embodiment, anti-BPI antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions, which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

Anti-BPI antibodies or fragments thereof can be conjugated to a therapeutic agent or drug moiety to modify a given biological response. The therapeutic agent or drug moiety is not to be construed as limited to classical chemical therapeutic agents, e.g., small molecules. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al., (eds.), pp. 243-56 (Alan R. Liss, Inc. 1985); Hellstrom et al., “Antibodies For Drug Delivery”, in Controlled Drug Delivery (2nd Ed.), Robinson et al., (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al., (eds.), pp. 475-506 (1985); “Analysis, Results, And Future Prospective Of The Therapeutic Use Of Radiolabelled Antibody In Cancer Therapy”, in Monoclonal Antibodies For Cancer Detection And Therapy, Baldwin et al., (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described in U.S. Pat. No. 4,676,980.

An antibody with or without a therapeutic moiety conjugated to it can be used as a therapeutic that is administered alone or in combination with cytokine(s).

Diagnosis of Breast Cancer

In accordance with the present invention, a first suitable biological sample e.g. tissue homogenate, serum, plasma or urine obtained from a subject suspected of having or known to have breast cancer can be used for diagnosis or monitoring. In one embodiment, a decreased abundance of one or more BFs or BPIs (or any combination of them) in a first sample or sample set relative to a second sample or sample set (from a subject or subjects free from breast cancer) or a previously determined reference range indicates the presence of primary or metastatic breast cancer. BFs and BPIs suitable for this purpose are described in Tables I and III and identified in Lists 1 and 5 and in Lists 3 and 7 as described in detail above.

In another embodiment of the invention, an increased abundance of one or more BFs or BPIs (or any combination of them) in a first sample or sample set compared to a second sample or sample set or a previously determined reference range indicates the presence of primary or metastatic breast cancer; BFs and BPIs suitable for this purpose are described in Tables I and III and identified in Lists 2 and 6 and in Lists 4 and 8, as described in detail above. In another embodiment, the relative abundance of one or more BFs or BPIs (or any combination of them) in a first sample or sample set compared to a second sample or sample set or a previously determined reference range indicates a stage of breast cancer (e.g., primary or metastatic breast cancer). In yet another embodiment, the relative abundance of one or more BFs or BPIs (or any combination of them) in a first sample or sample set relative to a second sample or sample set or a previously determined reference range indicates the degree or severity of breast cancer.

In a further embodiment, an aberrant level of one or more BFs or BPIs (or any combination of them) comprising a cluster, in a first sample or sample set relative to a second sample or sample set (from a subject or subjects free from breast cancer) or a previously determined reference range indicates the presence of breast cancer and more specifically, the type of breast cancer. Clusters of BFs and BPIs suitable for this purpose are described in Tables I and m identified in Clusters I-VI and described in detail in the examples (section 6.3).

In any of the aforesaid methods, detection of one or more BPIs described herein may optionally be combined with detection of one or more additional biomarkers for breast cancer including, but not limited to, the extracellular domain of the HER-2/neu oncogene product (Payne RC et al, Clin Chem 2000;46(2): 175-82). Any suitable method in the art can be employed to measure the level of BFs and BPIs, including but not limited to the Preferred Technology described herein, kinase assays, immunoassays to detect and/or visualise the BPIs (e.g., Western blot, immunoprecipitation followed by sodium dodecyl sulfate polyacrylamide gel electrophoresis, immunocytochemistry, etc.). In cases where a BPI has a known function, an assay for that function may be used to measure BPI expression. In a further embodiment, a decreased abundance of mRNA encoding one or more BPIs described in Table III and identified in List 5 or in List 7 (or any combination of BPIs within a given List) in a first sample or sample set relative to a second sample or sample set or a previously determined reference range indicates the presence of primary or metastatic breast cancer, respectively. In yet a further embodiment, an increased abundance of mRNA encoding one or more BPIs described in Table III and identified in List 6 or in List 8 (or any combination of BPIs within a given List) in a first sample or sample set relative to a second sample or sample set or previously determined reference range indicates the presence of primary or metastatic breast cancer, respectively. In another embodiment an aberrant level of mRNA encoding one or more BPIs comprising a cluster in a first sample or sample set relative to a second sample or sample set or a previously determined reference range indicates the presence of breast cancer and more specifically the type of breast cancer. Any suitable hybridisation assay can be used to detect BPI expression by detecting and/or visualising mRNA encoding the BPI (e.g., Northern assays, dot blots, in situ hybridisation, etc.).

In another embodiment of the invention, labelled antibodies, derivatives and analogs thereof, which specifically bind to a BPI can be used for diagnostic purposes to detect, diagnose, or monitor breast cancer. Preferably, breast cancer is detected in an animal, more preferably in a mammal and most preferably in a human.

Screening Assays

The invention provides methods for identifying agents (e.g., candidate agents) that bind to a BPI or have a stimulatory or inhibitory effect on the expression or activity of a BPI. The invention also provides methods of identifying agents or candidate agents that bind to a BPI fragment, BPI-related polypeptide or the BPI-fusion protein or have a stimulatory or inhibitory effect on the expression or activity of a BPI fragment, BPI-related polypeptide or a BPI fusion protein. Examples of agents or candidate agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12: 145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404406; Cwirla et al., 1990, Proc. Natl. Acad. Sci. USA 87:6378-6382; and Felici, 1991, J. Mol. Biol. 222:301-310).

In one embodiment, agents that interact with (i.e., bind to) a BPI, a BPI fragment (e.g. a functionally active fragment), a BPI-related polypeptide, or a BPI fusion protein are identified in a cell-based assay system. In accordance with this embodiment, cells expressing a BPI, a fragment of a BPI, a BPI-related polypeptide or a BPI fusion protein are contacted with a candidate agent or a control agent and the ability of the candidate agent to interact with the BPI is determined. If desired, this assay may be used to screen a plurality (e.g. a library) of candidate agents. The cell, for example, can be of prokaryotic origin (e.g., E. coli) or eukaryotic origin (e.g., yeast or mammalian). Further, the cells can express the BPI, fragment of the BPI, BPI-related polypeptide or a BPI fusion protein endogenously or be genetically engineered to express the BPI, fragment of the BPI, BPI-related polypeptide or a BPI fusion protein. In certain instances, the BPI, fragment of the BPI, BPI-related polypeptide, a fragment of the BPI-related polypeptide, or a BPI fusion protein or the candidate agent is labelled, for example with a radioactive label (such as 32P, 35S or 125I) or a fluorescent label (such as fluorescein isothiocyanate, rhodamine, phycoerythrin, phycocyanin, allophycocyanin, o-phthaldehyde or fluorescamine) to enable detection of an interaction between a BPI and a candidate agent. The ability of the candidate agent to interact directly or indirectly with a BPI, a fragment of a BPI, a BPI-related polypeptide, a fragment of a BPI-related polypeptide, or a BPI fusion protein can be determined by methods known to those of skill in the art. For example, the interaction between a candidate agent and a BPI, a fragment of a BPI, a BPI-related polypeptide or a BPI fusion protein can be determined by flow cytometry, a scintillation assay, immunoprecipitation or western blot analysis.

In another embodiment, agents that interact with (i.e., bind to) a BPI, a BPI fragment (e.g., a functionally active fragment) a BPI-related polypeptide, a fragment of a BPI-related polypeptide, or a BPI fusion protein are identified in a cell-free assay system. In accordance with this embodiment, a native or recombinant BPI or fragment thereof, or a native or recombinant BPI-related polypeptide or fragment thereof, or a BPI-fusion protein or fragment thereof, is contacted with a candidate agent or a control agent and the ability of the candidate agent to interact with the BPI or BPI-related polypeptide, or BPI fusion protein is determined. If desired, this assay may be used to screen a plurality (e.g. a library) of candidate agents. Preferably, the BPI, BPI fragment, BPI-related polypeptide or BPI-fusion protein is first immobilised, by, for example, contacting the BPI, BPI fragment, BPI-related polypeptide or a BPI fusion protein with an immobilised antibody which specifically recognises and binds it, or by contacting a purified preparation of the BPI, BPI fragment, BPI-related polypeptide or a BPI fusion protein with a surface designed to bind proteins. The BPI, BPI fragment, BPI-related polypeptide or a BPI fusion protein may be partially or completely purified (e.g., partially or completely free of other polypeptides) or part of a cell lysate. Further, the BPI, BPI fragment or BPI-related polypeptide may be a fusion protein comprising the BPI or a biologically active portion thereof, or BPI-related polypeptide and a domain such as glutathionine-S-transferase. Alternatively, the BPI, BPI fragment, BPI-related polypeptide or BPI fusion protein can be biotinylated using techniques well known to those of skill in the art (e.g., biotinylation kit, Pierce Chemicals; Rockford, Ill.). The ability of the candidate agent to interact with a BPI, BPI fragment, BPI-related polypeptide or a BPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a BPI or is responsible for the post-translational modification of a BPI. In a primary screen, a plurality (e.g., a library) of candidate agents are contacted with cells that naturally or recombinantly express: (i) a BPI, a BPI homolog a BPI-related polypeptide, a BPI fusion protein, or a biologically active fragment of any of the foregoing; and (ii) a protein that is responsible for processing of the BPI, BPI homolog, BPI-related polypeptide, BPI fusion protein, or fragment in order to identify agents that modulate the production, degradation, or post-translational modification of the BPI, BPI homolog, BPI-related polypeptide, BPI fusion protein or fragment. If desired, candidate agents identified in the primary screen can then be assayed in a secondary screen against cells naturally or recombinantly expressing the specific BPIs of interest. The ability of the candidate agent to modulate the production, degradation or post-translational modification of a BPI, homolog, BPI-related polypeptide, or BPI fusion protein can be determined by methods known to those of skill in the art, including without limitation, flow cytometry, a scintillation assay, immunoprecipitation and western blot analysis.

In another embodiment, agents that competitively interact with (i.e., bind to) a BPI, BPI fragment, BPI-related polypeptide or a BPI fusion protein are identified in a competitive binding assay. In accordance with this embodiment, cells expressing a BPI, BPI fragment, BPI-related polypeptide or a BPI fusion protein are contacted with a candidate agent and an agent known to interact with the BPI, BPI fragment, BPI-related polypeptide or a BPI fusion protein; the ability of the candidate agent to competitively interact with the BPI, BPI fragment, BPI-related polypeptide or a BPI fusion protein is then determined. Alternatively, agents that competitively interact with (i.e., bind to) a BPI, BPI fragment, BPI-related polypeptide or BPI fusion protein are identified in a cell-free assay system by contacting a BPI, BPI fragment, BPI-related polypeptide or a BPI fusion protein with a candidate agent and an agent known to interact with the BPI, BPI-related polypeptide or BPI fusion protein. As stated above, the ability of the candidate agent to interact with a BPI, BPI fragment, BPI-related polypeptide or a BPI fusion protein can be determined by methods known to those of skill in the art. These assays, whether cell-based or cell-free, can be used to screen a plurality (e.g., a library) of candidate agents.

In another embodiment, agents that modulate (i.e., upregulate or downregulate) the expression of a BPI, or a BPI-related polypeptide are identified by contacting cells (e.g., cells of prokaryotic origin or eukaryotic origin) expressing the BPI, or BPI-related polypeptide with a candidate agent or a control agent (e.g., phosphate buffered saline (PBS)) and determining the expression of the BPI, BPI-related polypeptide, or BPI fusion protein, mRNA encoding the BPI, or mRNA encoding the BPI-related polypeptide. The level of expression of a selected BPI, BPI-related polypeptide, mRNA encoding the BPI, or mRNA encoding the BPI-related polypeptide in the presence of the candidate agent is compared to the level of expression of the BPI, BPI-related polypeptide, mRNA encoding the BPI, or mRNA encoding the BPI-related polypeptide in the absence of the candidate agent (e.g., in the presence of a control agent). The candidate agent can then be identified as a modulator of the expression of the BPI, or a BPI-related polypeptide based on this comparison. For example, when expression of the BPI or mRNA is significantly greater in the presence of the candidate agent than in its absence, the candidate agent is identified as a stimulator of expression of the BPI or mRNA. Alternatively, when expression of the BPI or mRNA is significantly less in the presence of the candidate agent than in its absence, the candidate agent is identified as an inhibitor of the expression of the BPI or mRNA. The level of expression of a BPI or the mRNA that encodes it can be determined by methods known to those of skill in the art. For example, mRNA expression can be assessed by Northern blot analysis or RT-PCR, and protein levels can be assessed by western blot analysis.

The candidate agent may be an agonist or an antagonist of the BPI, BPI-related polypeptide, or BPI fusion protein, or of an upstream effector of the BPI, BPI-related polypeptide or BPI fusion protein.

In another embodiment, agents that modulate the activity of a BPI, or a BPI-related polypeptide are identified by contacting a preparation containing the BPI or BPI-related polypeptide, or cells (e.g., prokaryotic or eukaryotic cells) expressing the BPI or BPI-related polypeptide with a candidate agent or a control agent and determining the ability of candidate agent to modulate (e.g., stimulate or inhibit) the activity of the BPI or BPI-related polypeptide. The activity of a BPI or a BPI-related polypeptide can be assessed by detecting changes in a downstream effector such as, without limitation, induction of a cellular signal transduction pathway of the BPI or BPI-related polypeptide (e.g., intracellular Ca2′, diacylglycerol, IP3, etc.), detecting catalytic or enzymatic activity of the target on a suitable substrate, detecting the induction of a reporter gene (e.g., a regulatory element that is responsive to a BPI or a BPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639). The candidate agent can then be identified as a modulator of the activity of a BPI or BPI-related polypeptide by comparing the effects of the candidate agent to the control agent. Suitable control agents include phosphate buffered saline (PBS) and normal saline (NS).

In another embodiment, agents that modulate (i.e., upregulate or downregulate) the expression, activity or both the expression and activity of a BPI or BPI-related polypeptide are identified in an animal model. Examples of suitable animals include, but are not limited to, mice, rats, rabbits, monkeys, guinea pigs, dogs and cats Preferably, the animal model used represents a model of breast cancer (e.g., xenografts of human breast cancer cell lines such as MDA-MB-345 in estrogen-depreived Severe Combined Immunodeficient (SCID) mice, Eccles et al., 1994 Cell Biophysics 24/25, 279). In accordance with this embodiment, the candidate agent or a control agent is administered (e.g., orally, rectally or parenterally such as intraperitoneally or intravenously) to a suitable animal and the effect on the expression, activity or both expression and activity of the BPI or BPI-related polypeptide is determined. Changes in the expression of a BPI or BPI-related polypeptide can be assessed by the methods outlined above.

In yet another embodiment, a BPI or BPI-related polypeptide is used as a “bait protein” in a two-hybrid assay or three hybrid assay to identify other proteins that bind to or interact with a BPI or BPI-related polypeptide (see, e.g., U.S. Pat. No. 5,283,317; Zervos et al., (1993) Cell 72:223-232; Madura et al., (1993) J. Biol. Chem. 268:12046-12054; Bartel et al., (1993) Bio/Techniques 14:920-924; Iwabuchi et al., (1993) Oncogene 8:1693-1696; and WO 94/10300). As those skilled in the art will appreciate, such binding proteins may be involved in the propagation of signals by the BPIs of the invention as, for example, upstream or downstream elements of a signalling pathway involving the BPIs of the invention.

Suitable assays can be employed for detecting or quantifying the enzymatic or binding activity of a BPI, a BPI analog, a BPI-related polypeptide, or a fragment of any of the foregoing. In a preferred embodiment, as assay is used, for example, to screen for or identify a candidate agent that modulates the activity and or expression of a BPI, BPI analog, or BPI-related polypeptide, a fragment of any of the foregoing.

This invention further provides novel agents identified by the above-described screening assays and uses thereof for treatments as described herein.

Therapeutic Uses of BPIs

The invention provides for the treatment or prevention of breast cancer disease by administration of a therapeutic agent. Such agents include but are not limited to: BPIs, BPI analogs, BPI-related polypeptides and derivatives (including fragments) thereof; antibodies to the foregoing; nucleic acids encoding BPIs, BPI analogs, BPI-related polypeptides and fragments thereof; antisense nucleic acids to a gene encoding a BPI or BPI-related polypeptide; and modulator (e.g., agonists and antagonists) of a gene encoding a BPI or BPI-related polypeptide. An important feature of the present invention is the identification of genes encoding BPIs involved in primary and metastatic breast cancer. Breast cancer can be treated (e.g. to ameliorate symptoms or to retard onset or progression) or prevented by administration of a therapeutic agent that promotes function or expression of one or more BPIs that are decreased in the serum of breast cancer subjects having breast cancer, or by administration of a therapeutic agent that reduces function or expression of one or more BPIs that are increased in the serum of subjects having breast cancer.

In one embodiment, one or more antibodies each specifically binding to a BPI are administered alone or in combination with one or more additional therapeutic agents or treatments. Examples of such therapeutic compounds or treatments include, but are not limited to, taxol, cyclophosphamide, taxomifen, fluorouracil, doxorubicin.

Preferably, a biological product such as an antibody is allogeneic to the subject to which it is administered. In a preferred embodiment, a human BPI or a human BPI-related polypeptide, a nucleotide sequence encoding a human BPI or a human BPI-related polypeptide, or an antibody to a human BPI or a human BPI-related polypeptide, is administered to a human subject for therapy (e.g. to ameliorate symptoms or to retard onset or progression) or prophylaxis.

Treatment and Prevention of Breast Cancer

Breast cancer is treated or prevented by administration to a subject suspected of having or known to have breast cancer or to be at risk of developing breast cancer an agent that modulates (i.e. increases or decreases) the level or activity (i.e. function) of one or more BPIs, or the level of one or more BFs, that are differentially present in the serum of subjects having breast cancer compared with serum of subjects free from breast cancer. In one embodiment, breast cancer is treated or prevented by administering to a subject suspected of having or known to have breast cancer or to be at risk of developing breast cancer an agent that upregulates (i.e., increases) the level or activity (i.e., function) of one or more BPIs, or the level of one or more BFs, that are decreased in the serum of subjects having breast cancer. In another embodiment, an agent is administered that upregulates the level or activity (i.e., function) of one or more BPIs, or the level of one or more BFs, that are increased in the serum of subjects having breast cancer. Examples of such an agent include but are not limited to: BPIs, BPI fragments and BPI-related polypeptides; nucleic acids encoding a BPI, a BPI fragment and a BPI-related polypeptide (e.g., for use in gene therapy); and, for those BPIs or BPI-related polypeptides with enzymatic activity, agents or molecules known to modulate that enzymatic activity. Other agents that can be used, e.g., BPI agonists, can be identified using in vitro assays.

Breast cancer is also treated or prevented by administration to a subject suspected of having or known to have breast cancer or to be at risk of developing breast cancer an agent that downregulates the level or activity of one or more BPIs, or the level of one or more BFs, that are increased in the serum of subjects having breast cancer. In another embodiment, an agent is administered that downregulates the level or activity of one or more BPIs, or the level of one or more BFs, that are decreased in the serum of subjects having breast cancer. Examples of such an agent include, but are not limited to, BPI antisense oligonucleotides, ribozymes, antibodies directed against BPIs, and agents that inhibit the enzymatic activity of a BPI. Other useful agents e.g., BPI antagonists and small molecule BPI antagonists, can be identified using in vitro assays.

In a preferred embodiment, therapy or prophylaxis is tailored to the needs of an individual subject. Thus, in specific embodiments, agents that promote the level or function of one or more BPIs, or the level of one or more BFs, are therapeutically or prophylactically administered to a subject suspected of having or known to have breast cancer, in whom the levels or functions of said one or more BPIs, or levels of said one or more BFs, are absent or are decreased relative to a control or normal reference range. In further embodiments, agents that promote the level or function of one or more BPIs, or the level of one or more BFs, are therapeutically or prophylactically administered to a subject suspected of having or known to have breast cancer in whom the levels or functions of said one or more BPIs, or levels of said one or more BFs, are increased relative to a control or to a reference range. In further embodiments, agents that decrease the level or function of one or more BPIs, or the level of one or more BFs, are therapeutically or prophylactically administered to a subject suspected of having or known to have breast cancer in whom the levels or functions of said one or more BPIs, or levels of said one or more BFs, are increased relative to a control or to a reference range. In further embodiments, agents that decrease the level or function of one or more BPIs, or the level of one or more BFs, are therapeutically or prophylactically administered to a subject suspected of having or known to have breast cancer in whom the levels or functions of said one or more BPIs, or levels of said one or more BFs, are decreased relative to a control or to a reference range. The change in BPI function or level, or BF level, due to the administration of such agents can be readily detected, e.g., by obtaining a biological sample (e.g., a body fluid sample of blood or urine or a tissue sample such as biopsy tissue) and assaying in vitro the levels of said BFs or the levels or activities of said BPIs, or the levels of mRNAs encoding said BPIs or any combination of the foregoing. Such assays can be performed before and after the administration of the agent as described herein.

The agents of the invention include but are not limited to any agent, e.g., a small organic molecule, protein, peptide, antibody, nucleic acid, etc. that restores the breast cancer BPI or BF profile towards normal with the proviso that such agents do not include: cyclophophamide (Cytoxan); methotrexate (Methotrexate); 5-fluorouracil (5-FU); paclitaxel (Taxol); docetaxel (Taxotere); vincristine (Oncovin); vinblastine (Velban); vinorelbine (Navelbine); doxorubicin (Adriamycin); tamoxifen (Nolvadex); toremifene(Fareston); megestrol acetate(Megace); anastrozole (Arimidex); goserelin (Zoladex); trastuzumab (Herceptin); (Taxomifen); capecitabine (Xeloda.).

Gene Therapy

In a specific embodiment, nucleic acids comprising a sequence encoding a BPI, a BPI fragment, BPI-related polypeptide or fragment of a BPI-related polypeptide, are administered to promote BPI function by way of gene therapy. Gene therapy refers to administration to a subject of an expressed or expressible nucleic acid. In this embodiment, the nucleic acid produces its encoded polypeptide that mediates a therapeutic effect by promoting BPI function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al., (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the agent comprises a nucleic acid encoding a BPI or fragment or chimeric protein thereof, said nucleic acid being part of an expression vector that expresses a BPI or fragment or chimeric protein thereof in a suitable host. In particular, such a nucleic acid has a promoter operably linked to the BPI coding region, said promoter being inducible or constitutive (and, optionally, tissue-specific). In another particular embodiment, a nucleic acid molecule is used in which the BPI coding sequences and any other desired sequences are flanked by regions that promote homologous recombination at a desired site in the genome, thus providing for intrachromosomal expression of the BPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic™, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:44294432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., WO 92/06180, WO 92/22635, WO92/20316, WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains a nucleic acid encoding a BPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the BPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a BPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Direct injection of a DNA coding for a BPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a BPI and (b) a promoter are injected into a subject to elicit an immune response to the BPI.

Inhibition of BPIs to Treat Breast cancer

In one embodiment of the invention, breast cancer is treated or prevented by administration of an agent that antagonises (inhibits) the level(s) and/or function(s) of one or more BPIs which are elevated in a sample of subjects having breast cancer as compared with a sample of subjects free from breast cancer. Agents useful for this purpose include but are not limited to anti-BPI antibodies (and fragments and derivatives containing the binding region thereof), BPI antisense or ribozyme nucleic acids, and nucleic acids encoding dysfunctional BPIs that are used to “knockout” endogenous BPI function by homologous recombination (see, e.g., Capecchi, 1989, Science 244:1288-1292). Other agents that inhibit BPI function can be identified by use of known in vitro assays, e.g., assays for the ability of a test agent to inhibit binding of a BPI to another protein or a binding partner, or to inhibit a known BPI function. Preferably such inhibition is assayed in vitro or in cell culture, but genetic assays may also be employed. The Preferred Technology can also be used to detect levels of the BPIs before and after the administration of the agent. Preferably, suitable in vitro or in vivo assays are utilised to determine the effect of a specific agent and whether its administration is indicated for treatment of the affected tissue, as described in more detail below.

In a specific embodiment, an agent that inhibits a BPI function is administered therapeutically or prophylactically to a subject in whom an increased serum level or functional activity of the BPI (e.g., greater than the normal level or desired level) is detected as compared with serum of subjects free from breast cancer or a predetermined reference range. Methods standard in the art can be employed to measure the increase in a BPI level or function, as outlined above. Preferred BPI inhibitor compositions include small molecules, i.e., molecules of 1000 daltons or less. Such small molecules can be identified by the screening methods described herein.

Antisense Regulation of BPIs

In a specific embodiment, BPI expression is inhibited by use of BPI antisense nucleic acids. The present invention provides the therapeutic or prophylactic use of nucleic acids comprising at least six nucleotides that are antisense to a gene or cDNA encoding a BPI or a portion thereof. As used herein, a BPI “antisense” nucleic acid refers to a nucleic acid capable of hybridising by virtue of some sequence complementarity to a portion of an RNA (preferably mRNA) encoding a BPI. The antisense nucleic acid may be complementary to a coding and/or non-coding region of an mRNA encoding a BPI. Such antisense nucleic acids have utility as agents that inhibit BPI expression, and can be used in the treatment or prevention of breast cancer.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the BPI antisense nucleic acids of the invention in a pharmaceutically acceptable carrier, as described infra.

In another embodiment, the invention provides methods for inhibiting the expression of a BPI nucleic acid sequence in a prokaryotic or eukaryotic cell comprising providing the cell with an effective amount of a composition comprising a BPI antisense nucleic acid of the invention.

BPI antisense nucleic acids and their uses are described in detail below.

BPI Antisense Nucleic Acids

The BPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; WO 88/09810,) or blood-brain barrier (see, e.g., WO 89/10134,); hybridisation-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a BPI antisense oligonucleotide is provided, preferably of single-stranded DNA. The oligonucleotide may be modified at any position on its structure with substituents generally known in the art.

The BPI antisense oligonucleotide may comprise at least one of the following modified base moieties: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an alpha-anomeric oligonucleotide. An alpha-anomeric oligonucleotide forms specific double-stranded hybrids with complementary RNA in which, contrary to the usual alpha-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridisation-triggered cleavage agent.

Oligonucleotides of the invention may be synthesised by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the BPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the BPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the BPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a BPI, preferably a human gene encoding a BPI. However, absolute complementarity, although preferred, is not required. A sequence “complementary to at least a portion of an RNA,” as referred to herein, means a sequence having sufficient complementarity to be able to hybridise under stringent conditions (e.g., highly stringent conditions or moderately stringent conditions as defined supra) with the RNA, forming a stable duplex; in the case of double-stranded BPI antisense nucleic acids, a single strand of the duplex DNA may thus be tested, or triplex formation may be assayed. The ability to hybridise will depend on both the degree of complementarity and the length of the antisense nucleic acid. Generally, the longer the hybridising nucleic acid, the more base mismatches with an RNA encoding a BPI it may contain and still form a stable duplex (or triplex, as the case may be). One skilled in the art can ascertain a tolerable degree of mismatch by use of standard procedures to determine the melting point of the hybridised complex.

Therapeutic Use of BPI Antisense Nucleic Acids

The BPI antisense nucleic acids can be used to treat or prevent breast cancer when the target BPI is over-expressed in the serum of subjects suspected of having or suffering from breast cancer. In a preferred embodiment, a single-stranded DNA antisense BPI oligonucleotide is used.

Cell types which express or over-express RNA encoding a BPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridisation with a BPI-specific nucleic acid (e.g., by Northern hybridisation, dot blot hybridisation, in situ hybridisation), observing the ability of RNA from the cell type to be translated in vitro into a BPI, immunoassay, etc. In a preferred aspect, primary tissue from a subject can be assayed for BPI expression prior to treatment, e.g., by immunocytochemistry or in situ hybridisation.

Pharmaceutical compositions of the invention, comprising an effective amount of a BPI antisense nucleic acid in a pharmaceutically acceptable carrier, can be administered to a subject having breast cancer.

The amount of BPI antisense nucleic acid which will be effective in the treatment of breast cancer can be determined by standard clinical techniques.

In a specific embodiment, pharmaceutical compositions comprising one or more BPI antisense nucleic acids are administered via liposomes, microparticles, or microcapsules. In various embodiments of the invention, such compositions may be used to achieve sustained release of the BPI antisense nucleic acids.

Inhibitory Ribozyme and Triple Helix Approaches

In another embodiment, symptoms of breast cancer may be ameliorated by decreasing the level of a BPI or BPI activity by using gene sequences encoding the BPI in conjunction with well-known gene “knock-out”, ribozyme or triple helix methods to decrease gene expression of a BPI. In this approach ribozyme or triple helix molecules are used to modulate the activity, expression or synthesis of the gene encoding the BPI, and thus to ameliorate the symptoms of breast cancer. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding a BPI can be used to prevent translation of target gene mRNA and, therefore, expression of the gene product. (See, e.g., WO 90/11364; Sarver et al., 1990, Science 247:1222-1225).

Ribozymes are enzymatic RNA molecules capable of catalysing the specific cleavage of RNA (For a review, see Rossi, 1994, Current Biology 4, 469471). The mechanism of ribozyme action involves sequence specific hybridisation of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246.

While ribozymes that cleave mRNA at site-specific recognition sequences can be used to destroy mRNAs encoding a BPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591.

Preferably the ribozyme is engineered so that the cleavage recognition site is located near the 5′ end of the mRNA encoding the BPI, i.e., to increase efficiency and minimise the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridises to a target RNA sequence whereafter cleavage of the target RNA takes place. The invention encompasses those Cech-type ribozymes which target eight base-pair active site sequences that are present in the gene encoding the BPI.

As in the antisense approach, the ribozymes can be composed of modified oligonucleotides (e.g., for improved stability, targeting, etc.) and should be delivered to cells that express the BPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the BPI and inhibit translation. Because ribozymes, unlike antisense molecules, are catalytic, a lower intracellular concentration is required for efficacy.

Endogenous BPI expression can also be reduced by inactivating or “knocking out” the gene encoding the BPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435438). For example, a mutant gene encoding a non-functional BPI (or a completely unrelated DNA sequence) flanked by DNA homologous to the endogenous gene (either the coding regions or regulatory regions of the gene encoding the BPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, the endogenous expression of a gene encoding a BPI can be reduced by targeting deoxyribonucleotide sequences complementary to the regulatory region of the gene (i.e., the gene promoter and/or enhancers) to form triple helical structures that prevent transcription of the gene encoding the BPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesised in an alternating 5′-3′,3′-5′ manner, such that they base pair with first one strand of a duplex and then the other, eliminating the necessity for a sizeable stretch of either purines or pyrimidines to be present on one strand of a duplex.

In instances wherein the antisense, ribozyme, or triple helix molecules described herein are utilised to inhibit mutant gene expression, it is possible that the technique may so efficiently reduce or inhibit the transcription (triple helix) or translation (antisense, ribozyme) of mRNA produced by normal gene alleles of a BPI that the situation may arise wherein the concentration of BPI present may be lower than is necessary for a normal phenotype. In such cases, to ensure that substantially normal levels of activity of a gene encoding a BPI are maintained, gene therapy may be used to introduce into cells nucleic acid molecules that encode and express the BPI that exhibit normal gene activity and that do not contain sequences susceptible to whatever antisense, ribozyme, or triple helix treatments are being utilised. Alternatively, in instances whereby the gene encodes an extracellular protein, normal BPIs can be co-administered in order to maintain the requisite level of BPI activity.

Antisense RNA and DNA, ribozyme, and triple helix molecules of the invention may be prepared by any method known in the art for the synthesis of DNA and RNA molecules, as discussed above. These include techniques for chemically synthesising oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Therapeutic and Prophylactic Compositions and Their Use

The invention provides methods of treatment (and prophylaxis) comprising administering to a subject an effective amount of an active agent. An “active agent” as used herein comprises BPIs, BPI fragments, BPI-related polypeptides, anti-BPI antibodies, fragments of anti-BPI antibodies and agents which modulate the expression of BPIs e.g. agonists and antagonists of BPIs. In a preferred aspect, the agent is substantially purified (e.g., substantially free from substances that limit its effect or produce undesired side-effects). The subject is preferably an animal and is preferably a mammal, and most preferably human.

Formulations and methods of administration that can be employed when the agent comprises a nucleic acid are described above; additional appropriate formulations and routes of administration are described below. A “pharmaceutical composition” as used herein comprises an active agent optionally with a pharmaceutically acceptable carrier.

Various delivery systems are known and can be used to administer an agent of the invention, e.g., encapsulation in liposomes, microparticles, microcapsules, recombinant cells capable of expressing the agent, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolising agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibres. In one embodiment, administration can be by direct injection into tissue or body fluid or at the site (or former site) of cancerous tissue.

In another embodiment, the agent can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the agent can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, e.g. near the site of cancerous tissue for example, breast, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the agent of the invention is a nucleic acid encoding a protein, the nucleic acid can be administered in vivo as described supra.

The present invention also provides pharmaceutical compositions. Such compositions comprise a therapeutically effective amount of an agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences”, Ed. E. W. Martin, ISBN: 0-912734-04-3, Mack Publishing Co. Such compositions will contain a therapeutically effective amount of the agent, preferably in purified form, together with a suitable amount of carrier so as to provide the form for proper administration to the subject. The formulation should suit the mode of administration.

In a specific embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilising agent and a local anaesthetic such as lidocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the agent of the invention, which will be effective in the treatment of breast cancer can be determined by standard clinical techniques. In addition, in vitro assays may optionally be employed to help identify optimal dosage ranges. The precise dose to be employed in the formulation will also depend on the active agent, the route of administration of the active agent, and the seriousness of the disease or disorder, and should be decided according to the judgement of the practitioner and each subject's circumstances. However, suitable dosage ranges for intravenous administration are generally about 20-500 micrograms of active agent per kilogram body weight. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

When a reference is made herein to a method of treating or preventing a disease or condition using a particular agent or combination of agents, it is to be understood that such a reference is intended to include the use of that agent or combination of agents in the preparation of a medicament for the treatment or prevention of the disease or condition.

### EXAMPLE

### Identification of Proteins Differentially Expressed in Serum of Patients with Breast Cancer

Using the following exemplary and non-limiting procedure, proteins in serum samples from (a) 7 patients having primary breast cancer, (b) 5 patients having metastatic breast cancer, and (c) 8 unrelated control samples taken from subjects unaffected by breast cancer, were separated by isoelectric focusing followed by SDS-PAGE and then compared and analysed. Parts 6.1.1 to 6.1.14 (inclusive) of the procedure set forth below are hereby designated as the “Reference Protocol”.

Materials and Methods

Sample Preparation

A protein assay (Pierce BCA Cat # 23225) was performed on each serum sample as received. Prior to protein separation, each sample was processed for selective depletion of certain proteins, in order to enhance and simplify protein separation and facilitate analysis by removing proteins that may interfere with or limit analysis of proteins of interest, see WO 99/63351.

Removal of albumin, haptoglobin, transferrin and immunoglobulin G (IgG) from serum (“serum depletion”) was achieved by an affinity chromatography purification step in which the sample was passed through a series of Hi-Trap™ columns containing immobilised antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap™ columns (Protein G-Sepharose Hi-Trap™ columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“flow-through fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted serum containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (BDH 452043w)

4% CHAPS (Sigma C3023)

65 mM dithiothreitol (DTT)

2% (v/v) Resolytes 3.5-10 (BDH 44338 2x)

This mixture was vortexed, and centrifuged at 13000 rpm for 5 mins at 15° C., and the supernatant was separated by isoelectric focusing as described below.

Isoelectric Focusing

Isoelectric focusing (IEF), was performed using the Imrmobiline™ DryStrip Kit (Pharmacia BioTech), following the procedure described in the manufacturer's instructions, see Instructions for Immobiline™ DryStrip Kit, Pharmacia, # 18-1038-63, Edition AB (incorporated herein by reference in its entirety). Immobilised pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 ml of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs

For all stages of the process, the current limit was set to 10 mA for 12 gels, and the wattage limit to 5 W. The temperature was held at 20° C. throughout the run.

Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412423 (incorporated herein by reference in its entirety), with modifications as specified below.

Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of g-methacryl-oxypropyltrimethoxysilane in ethanol (BindSilane™; Pharmacia Cat. # 17-1330-01). The front plate was treated with a 2% solution of dimethyldichlorosilane dissolved in octamethyl cyclo-octasilane (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerisation. Casting was then carried out according to Hochstrasser et al., op. cit. A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerisation catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerise at 20° C. overnight, and then stored at 4° C. in sealed polyethylene bags with 6 ml of gel buffer, and were used within 4 weeks.

SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267. The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150 W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl]ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/l; the staining solution was vacuum filtered through a 0.41 μm filter (Duropore) before use.

Imaging of the Gel

A computer-readable output was produced by imaging the fluorescently stained gels with the preferred scanner described in section 5.2, supra. This scanner has a gel carrier with four integral fluorescent markers (Designated M1, M2, M3, M4) that are used to correct the image geometry and are a quality control feature to confirm that the scanning has been performed correctly. For scanning, the gels were removed from the stain, rinsed with water and allowed to air dry briefly, and imaged on the preferred scanner. After imaging, the gels were sealed in polyethylene bags containing a small volume of staining solution, and then stored at 4° C.

Digital Analysis of the Data

The data were processed as described in WO 98/23950 at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artefacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

Assignment of pI and MW Values

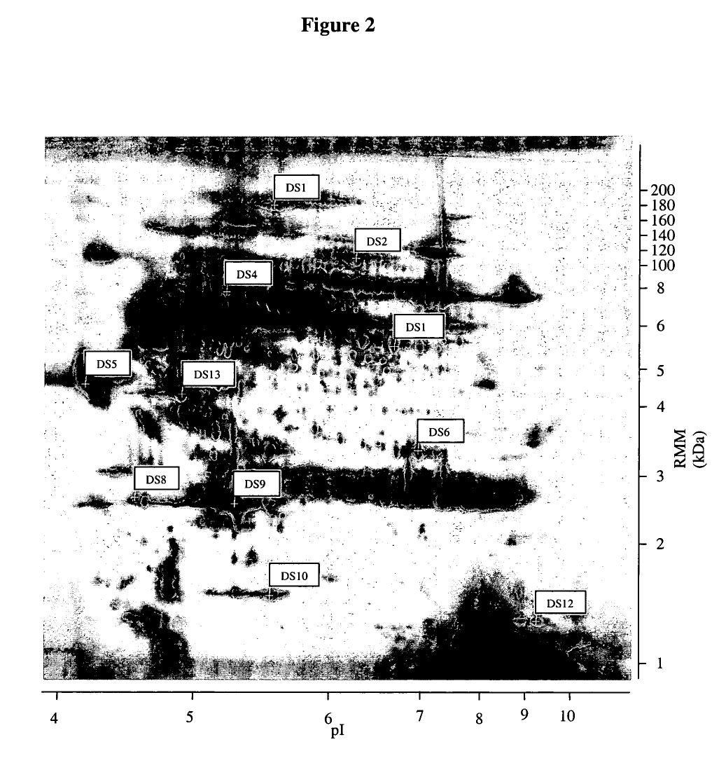

Landmark identification was used to determine the pI and MW of features detected in the images. Eleven landmark features, designated DS1, DS2, DS4, DS5, DS6, DS8, DS9, DS10, DS11, DS12, and DS 13 were identified in a standard serum image. These landmark features are identified in FIG. 2 and were assigned the pI and/or MW values identified in Table VI.

TABLE VII
Landmark Features Used in this Study

| Name | pI | MW (Da) | Name | pI | MW (Da) |
|---|---|---|---|---|---|
| DS1 | 5.55 | 18,5070 | DS9 | 5.22 | 23,000 |
| DS2 | 6.20 | 100,000 | DS10 | 5.52 | 13,800 |
| DS4 | 5.15 | 73,470 | DS11 | 6.65 | 56,170 |
| DS5 | 4.10 | 44,160 | DS12 | 9.01 | 12,060 |
| DS6 | 6.98 | 31,720 | DS13 | 4.75 | 41,230 |
| DS8 | 4.47 | 23,920 | | | |

As many of these landmarks as possible were identified in each gel image of the dataset. Each feature in the study gels was then assigned a pI value by linear interpolation or extrapolation (using the MELANIE™-II software) to the two nearest landmarks, and was assigned a MW value by linear interpolation or extrapolation (using the MELANIE™™-II software) to the two nearest landmarks.

Matching with Primary Master Image

Images were edited to remove gross artefacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modelling is that smooth signals may be modelled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modelled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognisably incorrect pairings were removed. Where the number of such recognisably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initialising the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y co-ordinates of the features within the gel, 3) the isoelectric point (pI) of the features, 4) the apparent molecular weight (MW) of the features, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

Statistical Analysis of the Profiles

All of the features (MCIs) identified from the samples were included in the analysis of the master gel image (refer to paragraph 0352)

In order to identify groups of BFs that would achieve a segregation of the samples that most closely matched the sub-type of breast cancer present, the master gel image was analysed using stepwise LDA. Given the size of the database and the number of “predictors”, an ab initio systematic and stepwise LDA was not practical. Instead, a uni-variate sub-group analysis was performed to identify those BFs that distinguished normal samples, primary breast cancer samples and metastatic breast cancer samples from each other. This was performed using the Wilcoxon Rank-Sum test, the MCIs that recorded a p-value of less than or equal to 0.05 were selected.

ERFs were present in all serum samples and the coefficient of variation was less than 10% across all samples.

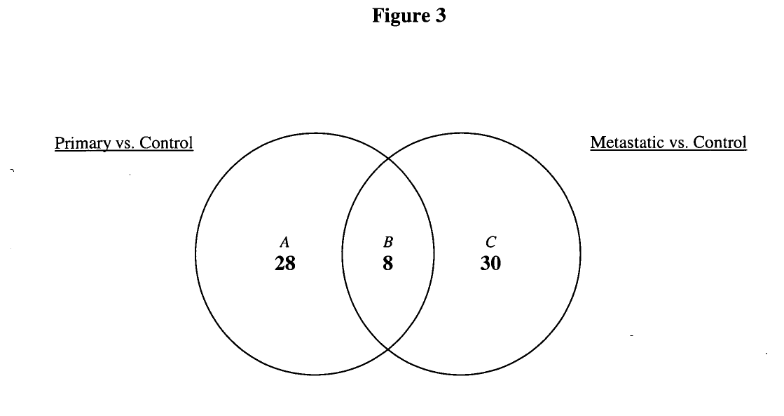

The MCIs (corresponding to BFs) selected were grouped according to the stage of breast cancer present i.e. primary breast cancer and metastatic breast cancer. The Venn diagram in FIG. 3 represents the diagnostic category of the BFs identified. Each Venn diagram position (A, B and C) lists a mutually exclusive set of BFs.

TABLE VIII
Description of the Venn diagram position and the diagnostic category

| Venn | |
|---|---|
| Diagram | |
| Position | Diagnostic condition |
| A | breast cancer specific markers for diagnosing |
| | individuals with primary breast cancer |
| B | breast cancer specific markers for diagnosing |
| | individuals with primary or metastatic breast cancer |
| C | breast cancer specific markers for diagnosing |
| | individuals with metastatic breast cancer |

A further subset of BFs was then selected on the basis of their prevalence in the sample group. This heuristic analysis yielded a subset of BFs on which several stepwise LDA were performed. The LDA results were validated using the leave-one-out cross validation method by observing the number of misclassifications. This multiple approach resulted in the identification of several clusters of BFs that can discriminate between samples from normal subjects, and patients with primary breast cancer and metastatic breast cancer.

Recovery and Analysis of Selected Proteins

Proteins in BFs were robotically excised and processed to generate tryptic digest peptides. Tryptic peptides were analysed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analysed by tandem mass spectrometry (MS/MS) using a Micromass Quadrupole Time-of-Flight (Q-TOF) mass spectrometer (Micromass, Altrincham, U.K.) equipped with a nanoflow™ electrospray Z-spray source. For partial amino acid sequencing and identification of BPIs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662). The method described WO 02/21139 was also used to interpret mass spectra.

Results

These initial experiments identified: 18 features that were decreased and 18 features that were increased in serum from 7 primary breast cancer patients as compared with serum from 8 patients unaffected by breast cancer; 24 features that were decreased and 13 features that were increased in the serum from 5 metastatic breast cancer patients as compared with serum from 8 patients unaffected by breast cancer. In addition, these experiments also provided 2 clusters of BFs which were able to distinguish between patients with primary breast cancer, patients with metastatic breast cancer and control subjects. Details of these BFs are provided in Table I. Each BF was differentially present in breast cancer serum as compared with normal serum (p<0.05). For some preferred BFs, BF-102, BF-116, BF-121, BF-126, BF-135, BF-144, BF-147, BF-151, BF-152, the difference was highly significant (p<0.01). The Venn diagram of FIG. 3 shows the overlap of BFs identified in different breast cancer stages. Details of the BFs present in each position of the Venn diagram together with the associated fold changes and p-values are given in Tables IX-XIV below.

TABLE IX
Decreased features in Venn Diagram Position A - Primary
Breast Cancer Specific Markers:

| BF# | pI | MW | Fold Change | p-value |
|---|---|---|---|---|
| BF-117 | 5.1 | 43858 | 4.06 | 0.030 |
| BF-126 | 5.7 | 27034 | 3.80 | 0.008 |
| BF-114 | 4.7 | 48182 | 2.37 | 0.020 |
| BF-103 | 5.6 | 153505 | 1.85 | 0.047 |
| BF-106 | 4.7 | 87712 | 1.70 | 0.037 |
| BF-128 | 5.7 | 22027 | 1.69 | 0.021 |
| BF-122 | 4.6 | 35101 | 1.68 | 0.042 |
| BF-127 | 6.9 | 23542 | 1.65 | 0.021 |
| BF-118 | 5.0 | 43942 | 1.61 | 0.047 |
| BF-112 | 5.3 | 48561 | 1.57 | 0.026 |
| BF-119 | 6.5 | 43553 | 1.41 | 0.035 |
| BF-111 | 6.6 | 53549 | 1.37 | 0.048 |
| BF-115 | 7.5 | 48169 | 1.28 | 0.037 |

TABLE X
Increased features in Venn Diagram Position A - Primary
Breast Cancer Specific Markers:

| BF# | pI | MW | Fold Change | p-value |
|---|---|---|---|---|
| BF-125 | 4.7 | 26996 | 2.37 | 0.037 |
| BF-110 | 4.9 | 55348 | 2.35 | 0.020 |
| BF-123 | 4.6 | 29031 | 2.34 | 0.020 |
| BF-121 | 6.0 | 36016 | 2.01 | 0.005 |
| BF-107 | 7.4 | 86906 | 2.01 | 0.028 |
| BF-120 | 4.8 | 43563 | 1.99 | 0.013 |
| BF-102 | 6.2 | 191412 | 1.95 | 0.009 |
| BF-101 | 5.1 | 192161 | 1.92 | 0.018 |
| BF-116 | 5.5 | 45867 | 1.71 | 0.005 |
| BF-113 | 6.9 | 48630 | 1.68 | 0.027 |
| BF-124 | 6.8 | 27312 | 1.56 | 0.018 |
| BF-108 | 6.1 | 78042 | 1.56 | 0.038 |
| BF-109 | 6.0 | 59414 | 1.50 | 0.047 |
| BF-105 | 7.2 | 109113 | 1.50 | 0.033 |
| BF-104 | 6.2 | 121435 | 1.42 | 0.043 |

TABLE XI
Decreased features in Venn Diagram Position B - Primary
and Metastatic Breast Cancer Specific Markers

| | | | Fold Change | p-value | Fold Change | p-value |
|---|---|---|---|---|---|---|
| BF# | pI | MW | Primary | Primary | Metastatic | Metastatic |
| BF-130 | 4.6 | 81856 | 2.14 | 0.038 | 3.32 | 0.008 |
| BF-132 | 5.2 | 61296 | 2.12 | 0.020 | 2.85 | 0.036 |
| BF-135 | 5.2 | 22982 | 1.45 | 0.003 | 1.60 | 0.004 |
| BF-131 | 5.8 | 69522 | 1.28 | 0.024 | 1.25 | 0.048 |
| BF-134 | 5.5 | 31784 | 1.20 | 0.024 | 1.89 | 0.004 |

TABLE XII
Increased features in Venn Diagram Position B - Primary
and Metastatic Breast Cancer Specific Markers:

| | | | Fold Change | p-value | Fold Change | p-value |
|---|---|---|---|---|---|---|
| BF# | pI | MW | Primary | Primary | Metastatic | Metastatic |
| BF-133 | 5.8 | 58171 | 3.47 | 0.014 | 2.24 | 0.025 |
| BF-136 | 7.1 | 15240 | 1.65 | 0.032 | 1.94 | 0.008 |
| BF-129 | 6.2 | 122600 | 1.45 | 0.030 | 2.06 | 0.022 |

TABLE XIII
Decreased features in Venn Diagram Position C - Metastatic
Breast Cancer Specific Markers:

| BF# | pI | MW | Fold Change | p-value |
|---|---|---|---|---|
| BF-145 | 5.2 | 63880 | 4.04 | 0.0231 |
| BF-149 | 4.5 | 59743 | 2.02 | 0.0338 |
| BF-150 | 5.5 | 53454 | 2.00 | 0.0481 |
| BF-137 | 5.9 | 196396 | 1.94 | 0.0478 |
| BF-139 | 7.5 | 119667 | 1.89 | 0.0157 |
| BF-159 | 5.7 | 31545 | 1.89 | 0.0338 |
| BF-142 | 5.2 | 86344 | 1.86 | 0.0338 |
| BF-143 | 4.8 | 86825 | 1.79 | 0.0230 |
| BF-147 | 5.0 | 64235 | 1.72 | 0.0068 |
| BF-157 | 7.7 | 34359 | 1.67 | 0.0472 |
| BF-160 | 6.7 | 27296 | 1.61 | 0.0338 |
| BF-166 | 8.7 | 12102 | 1.60 | 0.0200 |
| BF-165 | 6.0 | 15113 | 1.57 | 0.0252 |
| BF-155 | 6.1 | 43163 | 1.50 | 0.0481 |
| BF-162 | 6.7 | 24401 | 1.49 | 0.0338 |
| BF-148 | 5.0 | 62149 | 1.41 | 0.0231 |
| BF-164 | 5.5 | 22177 | 1.39 | 0.0127 |
| BF-146 | 5.3 | 63088 | 1.24 | 0.0481 |
| BF-158 | 6.3 | 32635 | 1.19 | 0.0279 |

TABLE XIV
Increased features in Venn Diagram Position C - Metastatic
Breast Cancer Specific Markers:

| BF# | pI | MW | Fold Change | p-value |
|---|---|---|---|---|
| BF-161 | 5.2 | 25593 | 5.20 | 0.0222 |
| BF-144 | 4.6 | 66507 | 3.34 | 0.0068 |
| BF-163 | 7.8 | 23857 | 2.35 | 0.0247 |
| BF-154 | 4.6 | 44519 | 2.31 | 0.0369 |
| BF-152 | 4.1 | 44095 | 2.02 | 0.0043 |
| BF-153 | 4.7 | 43530 | 1.91 | 0.0104 |
| BF-151 | 4.6 | 50129 | 1.77 | 0.0068 |
| BF-141 | 5.6 | 91596 | 1.71 | 0.0481 |
| BF-140 | 6.2 | 100014 | 1.55 | 0.0481 |
| BF-156 | 6.9 | 39900 | 1.53 | 0.0200 |
| BF-138 | 5.5 | 187242 | 1.38 | 0.0233 |

Examples of Clusters

The results obtained from the use of the Preferred Technology were subjected to multivariate analysis using the Linear Discriminant Approach described in Section 5.3.

Cluster I/III

This analysis identified a group of thirteen BFs that, taken as a group, showed a strong association with breast cancer. Alone, the individual BFs did not display a strong association with breast cancer (based on a uni-variate analysis). The group of BFs comprising the ‘cluster’ are listed in Table XV(a) below. Taken together these features displayed a statistical power that enabled normal, primary breast cancer, and metastatic breast cancer patient groupings to be discriminated from each other with an accuracy of 100% (i.e. with a misclassification rate of less than 0%).

Partial amino acid sequences were determined for the differentially present BPIs in these BFs. Details of these BPIs are also provided in Table XV(a).

TABLE XVa
The BFs and BPIs included in Cluster I/III

| BF | BPI | pI | MW (Da) | Tandem sequences |
|---|---|---|---|---|
| BF-108 | BPI-104 | 6.08 | 78042 | DDLYVSDAFHK, LPGIVAEGR, |
| | | | | EVPLNTIIFMGR, EQLQDMGLVDLFSPEK, |
| | | | | RVWELSK, |
| | BPI-103 | 6.08 | 78042 | ETAASLLQAGYK, |
| BF-132 | BPI-130 | 5.2 | 61296 | GDDITMVLILPKPEK, LPGIVAEGR, |
| | | | | VAEGTQVLELPFK, EQLQDMGLVDLFSPEK, |
| | | | | EVPLNTIIFMGR, RVWELSK, |
| BF-141 | BPI-149 | 5.63 | 91596 | DDLYVSDAFHK, LPGIVAEGR, |
| | | | | VAEGTQVLELPFK, RVWELSK, |
| | BPI-150 | 5.63 | 91596 | ETAASLLQAGYK, |
| BF-147 | BPI-158 | 5.03 | 64235 | LPGIVAEGR, EVPLNTIIFMGR, |
| BF-512 | BPI-521 | 5.57 | 92686 | DDLYVSDAFHK, LPGIVAEGR, RVWELSK, |
| BF-513 | BPI-523 | 5.96 | 79323 | DDLYVSDAFHK, LPGIVAEGR, |
| | | | | EVPLNTIIFMGR, |
| BF-514 | BPI-527 | 5.11 | 65901 | DDLYVSDAFHK, LPGIVAEGR, RVWELSK, |
| BF-515 | BPI-529 | 5.07 | 63379 | DDLYVSDAFHK, LPGIVAEGR, |
| | | | | EVPLNTIIFMGR, |
| BF-516 | BPI-531 | 5.17 | 61951 | LPGIVAEGR, EVPLNTIIFMGR, RVWELSK, |
| BF-517 | BPI-532 | 4.85 | 61074 | DDLYVSDAFHK, LPGIVAEGR, |
| BF-518 | BPI-533 | 5.34 | 60714 | DDLYVSDAFHK, VAEGTQVLELPFK, |
| | | | | EVPLNTIIFMGR, |
| BF-519 | BPI-534 | 5.3 | 57026 | RTHLPEVFLSK, THLPEVFLSK, YTFELSR, |
| | BPI-535 | 5.3 | 57026 | DDLYVSDAFHK, LPGIVAEGR, |
| BF-520 | BPI-536 | 4.96 | 46200 | DDLYVSDAFHK, VAEGTQVLELPFK, |

Table XV(b) shows for each BF comprising cluster I the ratio of BF abundance in breast cancer with respect to normal samples. The data shows how the subtypes of breast cancer (primary or metastatic) can be distinguished from control samples using the BFs which comprise cluster I.

TABLE XV(b)
Relative Abundance of BFs comprising cluster I in breast cancer.

| | Metastatic | Normal | Primary | Fold Change | |
|---|---|---|---|---|---|
| | Feature | Feature | Feature | Metastatic | Primary |
| | Presence | Presence | Presence | vs. | vs. |
| BF# | (%) | (%) | (%) | Normal | Normal |
| BF-132 | 100 | 75 | 85 | 2.85 | 2.12 |
| BF-151 | 100 | 100 | 100 | 1.77 | −1.16 |
| BF-157 | 80 | 87 | 85 | −1.67 | 1.33 |
| BF-509 | 40 | 62 | 100 | 1.36 | 1.25 |
| BF-510 | 60 | 37 | 28 | 1.65 | −1.84 |
| BF-511 | 60 | 62 | 28 | 2.31 | −1.00 |

Cluster II/IV

This analysis identified a group of six BFs that, taken as a group, showed a strong association with breast cancer. Alone, the individual BFs did not display a strong association with breast cancer (based on a uni-variate analysis).

The group of BFs comprising the ‘cluster’ are listed in Table XVI(a) below, taken together these features displayed a statistical power that enabled normal, primary breast cancer, and metastatic breast cancer patient groupings to be discriminated from each other with an accuracy of 100% (i.e. with a misclassification rate of less than 0%). Partial amino acid sequences were determined for the differentially present BPIs in these BFs. Details of these BPIs are provided in Table XVI(a).

TABLE XVI(a)
The BFs and BPIs in Cluster II/IV

| | | | | Amino Acid Sequences |
|---|---|---|---|---|
| BF | BPI | pI | MW (Da) | of Tryptic Digest Peptides |
| BF-132 | BPI-130 | 5.2 | 61296 | EQLQDMGLVDLFSPEK, |
| | | | | EVPLNTIIFMGR, |
| | | | | GDDITMVLILPKPEK, |
| | | | | LPGIVAEGR, RVWELSK, |
| | | | | VAEGTQVLELPFK |
| BF-151 | BPI-167 | 4.55 | 50129 | ENQLEVLEVSWLHGLK, |
| | | | | DLLLPQPDLR, |
| | | | | ALGHLDLSGNR, |
| | | | | VAAGAFQGLR, |
| | | | | YLFLNGNK, |
| BF-157 | BPI-173 | 7.66 | 34359 | ETAASLLQAGYK, |
| | | | | TATSEYQTFFNPR |
| | BPI-174 | 7.66 | 34359 | AYTNFDAER, TNQELQEINR, |
| | | | | AEDGSVIDYELIDQDAR, |
| | | | | SYSPYDMLESIR, |
| BF-509 | BPI-514 | 5.47 | 57934 | THLPEVFLSK, YTFELSR, |
| BF-510 | BPI-516 | 4.47 | 51499 | DLLLPQPDLR, |
| | | | | ALGHLDLSGNR, |
| | | | | VAAGAFQGLR, YLFLNGNK, |
| BF-511 | BPI-517 | 4.62 | 44998 | DLLLPQPDLR, YLFLNGNK, |

Table XV(b) shows for each BF comprising cluster II the ratio of BF abundance in breast cancer with respect to normal samples. The data shows how the subtypes of breast cancer (primary or metastatic) can be distinguished from control samples using the BFs which comprise cluster II.

TABLE XV(b)
Relative Abundance of BFs comprising cluster II in breast cancer.

| | Metastatic | Normal | Primary | Fold Change | |
|---|---|---|---|---|---|
| | Feature | Feature | Feature | Metastatic | Primary |
| | Presence | Presence | Presence | vs. | vs. |
| BF# | (%) | (%) | (%) | Normal | Normal |
| BF-108 | 80 | 75 | 100 | 1.14 | 1.56 |
| BF-132 | 100 | 75 | 85 | 2.85 | 2.12 |
| BF-141 | 100 | 100 | 100 | 1.71 | 1.21 |
| BF-147 | 100 | 100 | 100 | −1.72 | −1.12 |
| BF-512 | 100 | 100 | 100 | 1.32 | 1.05 |
| BF-513 | 100 | 100 | 100 | −1.63 | 1.33 |
| BF-514 | 20 | 12 | 0 | 1.75 | −7.33 |
| BF-515 | 40 | 37 | 57 | −1.09 | −1.75 |
| BF-516 | 100 | 87 | 100 | −2.05 | 1.06 |
| BF-517 | 100 | 100 | 100 | 1.03 | 1.35 |
| BF-518 | 0 | 37 | 28 | −8.48 | −1.80 |
| BF-519 | 100 | 100 | 85 | 1.45 | 1.18 |
| BF-520 | 100 | 87 | 57 | 1.39 | 1.19 |

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

Preferred features of each aspect of the invention are as for each of the other aspects mutatis mutandis.

We claim:

1. A method for screening or diagnosis of breast cancer in a subject, for determining the stage or severity of breast cancer in a subject, for identifying a subject at risk of developing breast cancer, or for monitoring the effect of therapy administered to a subject having breast cancer, said method comprising:
(a) analysing a test biological sample from the subject by two-dimensional electrophoresis to generate a two-dimensional array of features, said array comprising one or more of the following Breast Cancer Associated Features (BFs): BF-101, BF-102, BF-103, BF-104, BF-105, BF-106, BF-107, BF-108, BF-109, BF-110, BF-11, BF-112, BF-113, BF-114, BF-115, BF-116, BF-117, BF-118, BF-119, BF-120, BF-121, BF-122, BF-123, BF-124, BF-125, BF-126, BF-127, BF-128, BF-129, BF-130, BF-131, BF-132, BF-133, BF-134, BF-135, BF-136, BF-137, BF-138, BF-139, BF-140, BF-141, BF-142, BF-143, BF-144, BF-145, BF-146, BF-147, BF-148, BF-149, BF-150, BF-151, BF-152, BF-153, BF-155, BF-156, BF-157, BF-158, BF-159, BF-160, BF-161, BF-162, BF-163, BF-164, BF-165, BF-166, BF-509, BF-510, BF-511, BF-512, BF-513, BF-514, BF-515, BF-516, BF-517, BF-518, BF-519, BF-519, BF-520, and
(b) comparing the abundance of the one or more BFs in the test sample with the abundance of the one or more BFs in a biological sample from one or more subjects free from breast cancer, or with a previously determined reference range for that feature in subjects free from breast cancer, or with the abundance at least one Expression Reference Feature (ERF) in the test sample.

2. The method according to claim 1 wherein step b) comprises comparing the abundance of a cluster of BFs comprising the following: BF-108, BF-132, BF-141, BF-147, BF-512, BF-513, BF-514, BF-515, BF-516, BF-517, BF-518, BF-519, BF-520.

3. The method according to claim 1 wherein step b) comprises comparing the abundance of a cluster of BFs comprising the following: BF-132, BF-151, BF-157, BF-509, BF-510, BF-511.

4. A method for screening or diagnosis of breast cancer in a subject, for determining the stage or severity of breast cancer in a subject, for identifying a subject at risk of developing breast cancer, or for monitoring the effect of therapy administered to a subject having breast cancer, said method comprising quantitatively detecting, in a test biological sample from the subject, one or more of the following Breast Cancer Associated Protein Isoforms (BPIs): BPI-186, BPI-101, BPI-187, BPI-102, BPI-103, BPI-104, BPI-188, BPI-111, BPI-113, BPI-114, BPI-115, BPI-117, BPI-118, BPI-191, BPI-119, BPI-120, BPI-121, BPI-123, BPI-124, BPI-125, BPI-126, BPI-127, BPI-189, BPI-192, BPI-128, BPI-129, BPI-130, BPI-131, BPI-133, BPI-135, BPI-138, BPI-139, BPI-143, BPI-144, BPI-145, BPI-146, BPI-147, BPI-148, BPI-149, BPI-150, BPI-152, BPI-153, BPI-154, BPI-155, BPI-156, BPI-158, BPI-159, BPI-160, BPI-161, BPI-162, BPI-163, BPI-164, BPI-165, BPI-167, BPI-170, BPI-172, BPI-173, BPI-174, BPI-175, BPI-176, BPI-177, BPI-178, BPI-179, BPI-180, BPI-181, BPI-182, BPI-190, BPI-184, BPI-514, BPI-516, BPI-517, BPI-521, BPI-523, BPI-545, BPI-527, BPI-529, BPI-531, BPI-546, BPI-532, BPI-533, BPI-534, BPI-535, BPI-536.

5. The method according to claim 4 comprising quantitatively detecting a cluster of BPIs comprising the following: BPI-130, BPI-167, BPI-173, BPI-174, BPI-514, BPI-516, BPI-517.

6. The method according to claim 4 comprising quantitatively detecting a cluster of BPIs comprising the following: BPI-104, BPI-103, BPI-130, BPI-149, BPI-150, BPI-158, BPI-521, BPI-523, BPI-527, BPI-529, BPI-531, BPI-532, BPI-533, BPI-534, BPI-535, BPI-536.

7. The method according to claim 1 where the biological sample is serum or plasma.

8. The method according to claim 4 where the abundance of the one or more BPIs in the test sample is compared with the abundance of the one or more BPIs in a sample from one or more subjects free from breast cancer, or with a previously determined reference range for that feature in subjects free from breast cancer, or with the abundance at least one Expression Reference Feature (ERF) in the test sample.

9. The method according to claim 4, wherein the step of quantitatively detecting comprises testing at least one aliquot of the first sample, said step of testing comprising:
(a) contacting the aliquot with an antibody that is immunospecific for a BPI;
(b) quantitatively measuring the binding of the antibody and the BPI; and
(c) comparing the results of step (b) with a predetermined reference range.

10. The method according to claim 9, wherein the step of quantitatively detecting comprises testing a plurality of aliquots with a plurality of antibodies cognate for a plurality of preselected BPIs.

11. A pharmaceutical composition comprising a Breast Cancer Associated Protein Isoform (BPI) as defined in claim 4, or a nucleic acid encoding said BPI, and a pharmaceutically acceptable carrier.

12. The pharmaceutical composition according to claim 11, wherein the Breast Cancer Associated Protein Isoform (BPI) is in recombinant form.

13. An antibody capable of immunospecific binding to a Breast Cancer Associated Protein Isoform (BPI) as defined in claim 4.

14. The method according to claim 9, wherein the antibody is a monoclonal, chimeric, bispecific or humanised antibody.

15. The method according to claim 9, wherein the antibody binds to the BPI with greater affinity than to another isoform of the BPI.

16. A kit comprising one or more antibodies according to claim 13 and/or one or more BPIs as defined in claim 4, other reagents and instructions for use.

17. The kit of claim 16 for use in the screening or diagnosis of breast cancer in a subject, for determining the stage or severity of breast cancer in a subject, for identifying a subject at risk of developing breast cancer, or for monitoring the effect of therapy administered to a subject having breast cancer.

18. The kit according to claim 16 comprising a plurality of antibodies according to claim 13 and/or a plurality of BPIs as defined in claim 4.

19. A pharmaceutical composition comprising a therapeutically effective amount of an antibody, or a fragment or derivative of an antibody according to claim 13 and a pharmaceutically acceptable carrier.

20. A method of treating or preventing breast cancer comprising administering to a subject in need of such treatment a therapeutically effective amount of an antibody according to claim 13.

21. A method of treating or preventing breast cancer comprising administering to a subject in need of such treatment or prevention a therapeutically effective amount of one or more of the Breast Cancer Associated Protein Isoforms (BPIs) as defined in claim 4 and/or a nucleic acid encoding said BPIs.

22. A method of treating or preventing breast cancer comprising administering to a subject in need of such treatment or prevention a therapeutically effective amount of a nucleic acid that inhibits the function of one or more of the Breast Cancer Associated Protein Isoforms (BPIs) as defined in claim 4.

23. The method according to claim 22, wherein the nucleic acid is a BPI antisense nucleic acid or ribozyme.

24. A method of screening for agents that interact with one or more Breast Cancer Associated Protein Isoforms (BPIs) as defined in claim 4, fragments of BPIs (BPI fragment), polypeptides related to BPIs (BPI-related polypeptide), or BPI-fusion proteins said method comprising:
(a) contacting a BPI, a BPI fragment, a BPI-related polypeptide, or a BPI-fusion protein with a candidate agent; and
(b) determining whether or not the candidate agent interacts with the BPI, the BPI fragment, the BPI-related polypeptide, or the BPI-fusion protein.

25. The method according to claim 24, wherein the determination of interaction between the candidate agent and the BPI, BPI fragment, BPI-related polypeptide or BPI-fusion protein comprises quantitatively detecting binding of the candidate agent and the BPI, BPI fragment, BPI-related polypeptide or BPI-fusion protein.

26. A method of screening for or identifying agents that modulate the expression or activity of one or more Breast Cancer Associated Protein Isoforms (BPIs) as defined in claim 4, fragments of BPIs (BPI fragment), polypeptides related to BPIs (BPI-related polypeptide) or BPI-fusion proteins comprising:
(a) contacting a first population of cells expressing the BPI, BPI fragment, BPI-related polypeptide, or BPI-fusion protein with a candidate agent;
(b) contacting a second population of cells expressing said BPI, BPI fragment, BPI-related polypeptide, or BPI-fusion protein with a control agent; and
(c) comparing the level of said BPI, BPI fragment, BPI-related polypeptide, or BPI-fusion protein or mRNA encoding said BPI, BPI fragment, BPI-related polypeptide, or BPI-fusion protein in the first and second populations of cells, or comparing the level of induction of a downstream effector in the first and second populations of cells.

27. A method of screening for or identifying agents that modulate the expression or activity of one or more Breast Cancer Associated Protein Isoforms (BPIs) as defined in claim 4, fragments of BPIs (BPI fragment), polypeptides related to BPIs (BPI-related polypeptide) or BPI-fusion proteins said method comprising:
(a) administering a candidate agent to a first mammal or group of mammals;
(b) administering a control agent to a second mammal or group of mammals; and
(c) comparing the level of expression of the BPI, BPI fragment, BPI-related polypeptide or BPI-fusion protein, or mRNA encoding said BPI, BPI fragment, BPI-related polypeptide or BPI-fusion protein in the first and second groups, or comparing the level of induction of a downstream effector in the first and second groups.

28. The method as claimed in claim 27, wherein the mammals are animal models for breast cancer.

29. The method according to claim 27, wherein administration of a candidate agent results in an increase in the level of said BPI, BPI fragment, BPI-related polypeptide or BPI-fusion protein, or mRNA encoding said BPI, BPI fragment, BPI-related polypeptide, or BPI-fusion protein, or said downstream effector in the first population of cells or mammals compared to the second population of cells or mammals.

30. The method according to claim 27, wherein administration of a candidate agent results in a decrease in the level of said BPI, BPI fragment, BPI-related polypeptide, or BPI-fusion protein, or mRNA encoding said BPI, BPI fragment, BPI-related polypeptide, or BPI-fusion protein, or said downstream effector in the first population of cells or mammals compared to the second population of cells or mammals.

31. The method according to claim 27, wherein the levels of said BPI, BPI fragment, BPI-related polypeptide, or BPI-fusion protein, or mRNA encoding said BPI, BPI fragment, BPI-related polypeptide, or BPI-fusion protein, or of said downstream effector in the first and second groups are further compared to the level of said BPI, BPI fragment, BPI-related polypeptide or BPI-fusion protein, or mRNA encoding said BPI, BPI fragment, BPI-related polypeptide or BPI-fusion protein in normal control mammals.

32. The method according to claim 31, wherein said mammals are human subjects with breast cancer.

33. A method of screening for or identifying agents that modulate the activity of one or more of the Breast Cancer Associated Proteins Isoforms (BPIs) as defined in claim 4, fragments of BPIs (BPI fragment), polypeptides related to BPIs (BPI-related polypeptide) or BPI-fusion proteins said method comprising:
(a) in a first aliquot, contacting a candidate agent with the BPI, BPI fragment, BPI-related polypeptide or BPI-fusion protein, and
(b) determining and comparing the activity of the BPI, BPI fragment, BPI-related polypeptide or BPI-fusion protein in the first aliquot after addition of the candidate agent with the activity of the BPI, BPI fragment, BPI-related polypeptide or BPI-fusion protein in a control aliquot, or with a previously determined reference range.

34. The method according to claim 24, wherein the BPI, BPI fragment, BPI-related polypeptide, or BPI-fusion protein is a recombinant protein.

35. The method according to claim 24, wherein the BPI, BPI fragment, BPI-related polypeptide or BPI-fusion protein is immobilised on a solid phase.

36. A method for screening or diagnosis of breast cancer in a subject or for monitoring the effect of an anti-breast cancer drug or therapy administered to a subject, comprising:
(a) contacting at least one oligonucleotide probe comprising 10 or more consecutive nucleotides complementary to a nucleotide sequence encoding a BPI as defined in claim 4 with RNA obtained from a biological sample from the subject or with cDNA copied from the RNA wherein said contacting occurs under conditions that permit hybridisation of the probe to the nucleotide sequence if present;
(b) detecting hybridisation, if any, between the probe and the nucleotide sequence; and
(c) comparing the hybridisation, if any, detected in step (b) with the hybridisation detected in a control sample, or with a previously determined reference range.

37. The method as claimed in claim 36, wherein step (a) includes the step of hybridising the nucleotide sequence to a DNA array, wherein one or more members of the array are the probes complementary to a plurality of nucleotide sequences encoding distinct BPIs.

38. A method of modulating the activity of one or more of the Breast Cancer Associated Protein Isoforms as defined in claim 4 comprising administering to a subject an agent identified by claim 24.

39. A method of treating or preventing breast cancer comprising administering to a subject in need of such treatment or prevention a therapeutically effective dose of an agent that modulates the activity of one or more of the Breast Cancer Associated Protein Isoforms as defined in claim 4; whereby the symptoms of the breast cancer are ameliorated.

40. A method for identifying targets for therapeutic modulation of breast cancer wherein the activity of one or more of the Breast Cancer Associated Protein Isoforms as defined in claim 4 is utilised as a measure to determine whether a candidate target is effective for modulation of breast cancer.


SEQUENCE LISTING (156 sequences)

SEQ ID NO: 1 - protein, 13 aa, Homo sapiens
AADDTWEPFASGK

SEQ ID NO: 2 - protein, 19 aa, Homo sapiens
AAPSVTLFPPSSEELQANK

SEQ ID NO: 3 - protein, 10 aa, Homo sapiens
ADLSGITGAR

SEQ ID NO: 4 - protein, 17 aa, Homo sapiens
AEDGSVIDYELIDQDAR

SEQ ID NO: 5 - protein, 13 aa, Homo sapiens
AEMADQAAAWLTR

SEQ ID NO: 6 - protein, 13 aa, Homo sapiens
AFTECCVVASQLR

SEQ ID NO: 7 - protein, 11 aa, Homo sapiens
AIGYLNTGYQR

SEQ ID NO: 8 - protein, 9 aa, Homo sapiens
AKPALEDLR

SEQ ID NO: 9 - protein, 11 aa, Homo sapiens
ALGHLDLSGNR

SEQ ID NO: 10 - protein, 16 aa, Homo sapiens
ALNHLPLEYNSALYSR

SEQ ID NO: 11 - protein, 12 aa, Homo sapiens
ALQDQLVLVAAK

SEQ ID NO: 12 - protein, 10 aa, Homo sapiens
ALVQQMEQLR

SEQ ID NO: 13 - protein, 10 aa, Homo sapiens
APHGPGLIYR

SEQ ID NO: 14 - protein, 12 aa, Homo sapiens
ASSIIDELFQDR

SEQ ID NO: 15 - protein, 9 aa, Homo sapiens
ATVVYQGER

SEQ ID NO: 16 - protein, 19 aa, Homo sapiens
AVLDVFEEGTEASAATAVK

SEQ ID NO: 17 - protein, 7 aa, Homo sapiens
AVLYNYR

SEQ ID NO: 18 - protein, 9 aa, Homo sapiens
AYTNFDAER

SEQ ID NO: 19 - protein, 15 aa, Homo sapiens
DASGVTFTWTPSSGK

SEQ ID NO: 20 - protein, 11 aa, Homo sapiens
DDLYVSDAFHK

SEQ ID NO: 21 - protein, 10 aa, Homo sapiens
DEPPQSPWDR

SEQ ID NO: 22 - protein, 8 aa, Homo sapiens
DGFFGNPR

SEQ ID NO: 23 - protein, 15 aa, Homo sapiens
DGFVQDEGTMFPVGK

SEQ ID NO: 24 - protein, 10 aa, Homo sapiens
DLHLSDVFLK

SEQ ID NO: 25 - protein, 10 aa, Homo sapiens
DLLLPQPDLR

SEQ ID NO: 26 - protein, 12 aa, Homo sapiens
DPTFIPAPIQAK

SEQ ID NO: 27 - protein, 12 aa, Homo sapiens
DTGTYGFLLPER

SEQ ID NO: 28 - protein, 14 aa, Homo sapiens
DYLLLVMEGTDDGR

SEQ ID NO: 29 - protein, 9 aa, Homo sapiens
EFDHNSNIR

SEQ ID NO: 30 - protein, 10 aa, Homo sapiens
EGMLSIMSYR

SEQ ID NO: 31 - protein, 9 aa, Homo sapiens
EIGELYLPK

SEQ ID NO: 32 - protein, 11 aa, Homo sapiens
EIMENYNIALR

SEQ ID NO: 33 - protein, 18 aa, Homo sapiens
EIVLTQSPGTLSLSPGER

SEQ ID NO: 34 - protein, 17 aa, Homo sapiens
ELTTEIDNNIEQISSYK

SEQ ID NO: 35 - protein, 16 aa, Homo sapiens
ENQLEVLEVSWLHGLK

SEQ ID NO: 36 - protein, 14 aa, Homo sapiens
EQLGEFYEALDCLR

SEQ ID NO: 37 - protein, 16 aa, Homo sapiens
EQLGPVTQEFWDNLEK

SEQ ID NO: 38 - protein, 16 aa, Homo sapiens
EQLQDMGLVDLFSPEK

SEQ ID NO: 39 - protein, 11 aa, Homo sapiens
ESYSGVTLDPR

SEQ ID NO: 40 - protein, 12 aa, Homo sapiens
ETAASLLQAGYK

SEQ ID NO: 41 - protein, 11 aa, Homo sapiens
ETLFSVMPGLK

SEQ ID NO: 42 - protein, 8 aa, Homo sapiens
ETLLQDFR

SEQ ID NO: 43 - protein, 12 aa, Homo sapiens
EVPLNTIIFMGR

SEQ ID NO: 44 - protein, 12 aa, Homo sapiens
EVVADSVWVDVK

SEQ ID NO: 45 - protein, 12 aa, Homo sapiens
EWFWDLATGTMK

SEQ ID NO: 46 - protein, 9 aa, Homo sapiens
FAHTVVTSR

SEQ ID NO: 47 - protein, 12 aa, Homo sapiens
FLVGPDGIPIMR

SEQ ID NO: 48 - protein, 10 aa, Homo sapiens
FPGQLNADLR

SEQ ID NO: 49 - protein, 9 aa, Homo sapiens
FPPEEELQR

SEQ ID NO: 50 - protein, 13 aa, Homo sapiens
FSGSGSGTDFTLK

SEQ ID NO: 51 - protein, 16 aa, Homo sapiens
FSGSNSGNTATLTISR

SEQ ID NO: 52 - protein, 7 aa, Homo sapiens
FTFEYSR

SEQ ID NO: 53 - protein, 10 aa, Homo sapiens
FVTWIEGVMR

SEQ ID NO: 54 - protein, 15 aa, Homo sapiens
GDDITMVLILPKPEK

SEQ ID NO: 55 - protein, 11 aa, Homo sapiens
GEPGDPVNLLR

SEQ ID NO: 56 - protein, 9 aa, Homo sapiens
GFVVAGPSR

SEQ ID NO: 57 - protein, 8 aa, Homo sapiens
GIYGTISR

SEQ ID NO: 58 - protein, 14 aa, Homo sapiens
GQGTLSVVTMYHAK

SEQ ID NO: 59 - protein, 13 aa, Homo sapiens
GSPAINVAVHVFR

SEQ ID NO: 60 - protein, 10 aa, Homo sapiens
GYSIFSYATK

SEQ ID NO: 61 - protein, 9 aa, Homo sapiens
HHGPTITAK

SEQ ID NO: 62 - protein, 11 aa, Homo sapiens
HSIFTPETNPR

SEQ ID NO: 63 - protein, 10 aa, Homo sapiens
HTLNQIDEVK

SEQ ID NO: 64 - protein, 12 aa, Homo sapiens
HYDGSYSTFGER

SEQ ID NO: 65 - protein, 9 aa, Homo sapiens
IDVHLVPDR

SEQ ID NO: 66 - protein, 11 aa, Homo sapiens
INHGILYDEEK

SEQ ID NO: 67 - protein, 14 aa, Homo sapiens
IPIEDGSGEVVLSR

SEQ ID NO: 68 - protein, 9 aa, Homo sapiens
IPTTFENGR

SEQ ID NO: 69 - protein, 10 aa, Homo sapiens
ISEQFTAMFR

SEQ ID NO: 70 - protein, 8 aa, Homo sapiens
ISVIRPSK

SEQ ID NO: 71 - protein, 11 aa, Homo sapiens
ITLLSALVETR

SEQ ID NO: 72 - protein, 9 aa, Homo sapiens
IVQLIQDTR

SEQ ID NO: 73 - protein, 7 aa, Homo sapiens
IWDVVEK

SEQ ID NO: 74 - protein, 14 aa, Homo sapiens
KAADDTWEPFASGK

SEQ ID NO: 75 - protein, 10 aa, Homo sapiens
KATVVYQGER

SEQ ID NO: 76 - protein, 13 aa, Homo sapiens
KVTYTSQEDLVEK

SEQ ID NO: 77 - protein, 10 aa, Homo sapiens
LALDNGGLAR

SEQ ID NO: 78 - protein, 9 aa, Homo sapiens
LEPYADQLR

SEQ ID NO: 79 - protein, 7 aa, Homo sapiens
LHELQEK

SEQ ID NO: 80 - protein, 9 aa, Homo sapiens
LLIYDTSNR

SEQ ID NO: 81 - protein, 9 aa, Homo sapiens
LLIYGASSR

SEQ ID NO: 82 - protein, 9 aa, Homo sapiens
LPGIVAEGR

SEQ ID NO: 83 - protein, 11 aa, Homo sapiens
LPPNVVEESAR

SEQ ID NO: 84 - protein, 10 aa, Homo sapiens
LQGTLPVEAR

SEQ ID NO: 85 - protein, 14 aa, Homo sapiens
LVAYYTLIGASGQR

SEQ ID NO: 86 - protein, 13 aa, Homo sapiens
LVHVEEPHTETVR

SEQ ID NO: 87 - protein, 18 aa, Homo sapiens
LYGSEAFATDFQDSAAAK

SEQ ID NO: 88 - protein, 7 aa, Homo sapiens
NEQVEIR

SEQ ID NO: 89 - protein, 8 aa, Homo sapiens
NGFYPATR

SEQ ID NO: 90 - protein, 11 aa, Homo sapiens
NILTSNNIDVK

SEQ ID NO: 91 - protein, 15 aa, Homo sapiens
NWGLSVYADKPETTK

SEQ ID NO: 92 - protein, 9 aa, Homo sapiens
QDGSVDFFR

SEQ ID NO: 93 - protein, 11 aa, Homo sapiens
QDNEILIFWSK

SEQ ID NO: 94 - protein, 14 aa, Homo sapiens
QEPGENSEILPTLK

SEQ ID NO: 95 - protein, 17 aa, Homo sapiens
QEPSQGTTTFAVTSILR

SEQ ID NO: 96 - protein, 8 aa, Homo sapiens
QIQVSWLR

SEQ ID NO: 97 - protein, 8 aa, Homo sapiens
QLEWGLER

SEQ ID NO: 98 - protein, 11 aa, Homo sapiens
QLNEINYEDHK

SEQ ID NO: 99 - protein, 17 aa, Homo sapiens
QPFVQGLALYTPVVLPR

SEQ ID NO: 100 - protein, 11 aa, Homo sapiens
QPVPGQQMTLK

SEQ ID NO: 101 - protein, 8 aa, Homo sapiens
RLWWLDLK

SEQ ID NO: 102 - protein, 10 aa, Homo sapiens
RPYFPVAVGK

SEQ ID NO: 103 - protein, 12 aa, Homo sapiens
RQDNEILIFWSK

SEQ ID NO: 104 - protein, 11 aa, Homo sapiens
RTHLPEVFLSK

SEQ ID NO: 105 - protein, 11 aa, Homo sapiens
RVEPYGENFNK

SEQ ID NO: 106 - protein, 7 aa, Homo sapiens
RVWELSK

SEQ ID NO: 107 - protein, 9 aa, Homo sapiens
SAVQGPPER

SEQ ID NO: 108 - protein, 9 aa, Homo sapiens
SDVVYTDWK

SEQ ID NO: 109 - protein, 11 aa, Homo sapiens
SEYGAALAWEK

SEQ ID NO: 110 - protein, 16 aa, Homo sapiens
SGAQATWTELPWPHEK

SEQ ID NO: 111 - protein, 12 aa, Homo sapiens
SGSDEVQVGQQR

SEQ ID NO: 112 - protein, 9 aa, Homo sapiens
SIPQVSPVR

SEQ ID NO: 113 - protein, 17 aa, Homo sapiens
SLAELGGHLDQQVEEFR

SEQ ID NO: 114 - protein, 13 aa, Homo sapiens
SLDFTELDVAAEK

SEQ ID NO: 115 - protein, 16 aa, Homo sapiens
SNLDEDIIAEENIVSR

SEQ ID NO: 116 - protein, 16 aa, Homo sapiens
SPEQQETVLDGNLIIR

SEQ ID NO: 117 - protein, 12 aa, Homo sapiens
SYSPYDMLESIR

SEQ ID NO: 118 - protein, 13 aa, Homo sapiens
TATSEYQTFFNPR

SEQ ID NO: 119 - protein, 12 aa, Homo sapiens
TEQWSTLPPETK

SEQ ID NO: 120 - protein, 11 aa, Homo sapiens
THLAPYSDELR

SEQ ID NO: 121 - protein, 10 aa, Homo sapiens
THLPEVFLSK

SEQ ID NO: 122 - protein, 12 aa, Homo sapiens
TIYTPGSTVLYR

SEQ ID NO: 123 - protein, 11 aa, Homo sapiens
TLLPVSKPEIR

SEQ ID NO: 124 - protein, 10 aa, Homo sapiens
TNQELQEINR

SEQ ID NO: 125 - protein, 11 aa, Homo sapiens
TQVNTQAEQLR

SEQ ID NO: 126 - protein, 18 aa, Homo sapiens
TVAAPSVFIFPPSDEQLK

SEQ ID NO: 127 - protein, 10 aa, Homo sapiens
VAAGAFQGLR

SEQ ID NO: 128 - protein, 13 aa, Homo sapiens
VAEGTQVLELPFK

SEQ ID NO: 129 - protein, 9 aa, Homo sapiens
VASYGVKPR

SEQ ID NO: 130 - protein, 17 aa, Homo sapiens
VGEVLNSIFFELEADER

SEQ ID NO: 131 - protein, 11 aa, Homo sapiens
VGFYESDVMGR

SEQ ID NO: 132 - protein, 16 aa, Homo sapiens
VHQYFNVELIQPGAVK

SEQ ID NO: 133 - protein, 9 aa, Homo sapiens
VLFYVDSEK

SEQ ID NO: 134 - protein, 15 aa, Homo sapiens
VLSLAQEQVGGSPEK

SEQ ID NO: 135 - protein, 10 aa, Homo sapiens
VQPYLDDFQK

SEQ ID NO: 136 - protein, 8 aa, Homo sapiens
VRPQQLVK

SEQ ID NO: 137 - protein, 8 aa, Homo sapiens
VSVFVPPR

SEQ ID NO: 138 - protein, 11 aa, Homo sapiens
VTIGLLFWDGR

SEQ ID NO: 139 - protein, 12 aa, Homo sapiens
VTYTSQEDLVEK

SEQ ID NO: 140 - protein, 8 aa, Homo sapiens
VWVYPPEK

SEQ ID NO: 141 - protein, 13 aa, Homo sapiens
VYAYYNLEESCTR

SEQ ID NO: 142 - protein, 14 aa, Homo sapiens
WEMPFDPQDTHQSR

SEQ ID NO: 143 - protein, 9 aa, Homo sapiens
WFYIASAFR

SEQ ID NO: 144 - protein, 10 aa, Homo sapiens
WLQGSQELPR

SEQ ID NO: 145 - protein, 9 aa, Homo sapiens
WQEEMELYR

SEQ ID NO: 146 - protein, 15 aa, Homo sapiens
YAASSYLSLTPEQWK

SEQ ID NO: 147 - protein, 7 aa, Homo sapiens
YEFLNGR

SEQ ID NO: 148 - protein, 8 aa, Homo sapiens
YENEVALR

SEQ ID NO: 149 - protein, 10 aa, Homo sapiens
YEVQGEVFTK

SEQ ID NO: 150 - protein, 15 aa, Homo sapiens
YEVQGEVFTKPQLWP

SEQ ID NO: 151 - protein, 9 aa, Homo sapiens
YGIDWASGR

SEQ ID NO: 152 - protein, 8 aa, Homo sapiens
YLFLNGNK

SEQ ID NO: 153 - protein, 7 aa, Homo sapiens
YLTWASR

SEQ ID NO: 154 - protein, 7 aa, Homo sapiens
YTFELSR

SEQ ID NO: 155 - protein, 18 aa, Homo sapiens
YVLTQPPSVSVAPGQTAR

SEQ ID NO: 156 - protein, 15 aa, Homo sapiens
YVTSAPMPEPQAPGR